(19) United States
(12) Patent Application Publication
(10) Pub. No.: US 2024/0245324 A1
(43) Pub. Date: Jul. 25, 2024

(54) SYSTEMS, DEVICES, AND METHODS FOR ANALYTE MONITORING

(71) Applicant: ABBOTT DIABETES CARE INC., Alameda, CA (US)

(72) Inventors: Mario S. Pace, San Carlos, CA (US); Steven T. Mitchell, Pleasant Hill, CA (US); Matthew Simmons, Pleasanton, CA (US); Louis G. Pace, San Carlos, CA (US)

(21) Appl. No.: 18/414,677

(22) Filed: Jan. 17, 2024

Related U.S. Application Data
(60) Provisional application No. 63/439,663, filed on Jan. 18, 2023.

(51) Int. Cl. A61B 5/145

(52) U.S. Cl. CPC A61B 5/14532; A61B 5/14503

(57) ABSTRACT

Systems, devices, and methods are provided for inserting a sensor into skin of a subject. The insertion apparatus includes a sensor control device, a housing, and a guide for guiding insertion of a sensor into the skin. The housing may have an interface element, a cover, and a device carrier configured to releasably retain the sensor control device. The guide may be configured to support at least a portion of the sensor, and includes a retaining element. In a first configuration, the retaining element is configured to engage with the interface element to resist distal movement of the housing relative to the guide. In a second configuration, the interface element is configured to disengage from the retaining element to distally move the housing relative to the guide and insert the sensor into the skin.

### CROSS-REFERENCE TO RELATED APPLICATIONS

This application claims priority to U.S. Provisional Application No. 63/439,663, filed Jan. 18, 2023, which is herein expressly incorporated by reference in its entirety for all purposes.

### FIELD

The subject matter described herein relates generally to systems, devices, and methods for in vivo analyte monitoring. In particular, the present disclosure relates to an insertion apparatus for inserting a sensor into skin of a subject.

### BACKGROUND

The detection and/or monitoring of analyte levels, such as glucose, ketones, lactate, oxygen, hemoglobin AIC, or the like, can be vitally important to the overall health of a person, particularly for an individual having diabetes. Patients suffering from diabetes mellitus can experience complications including loss of consciousness, cardiovascular disease, retinopathy, neuropathy, and nephropathy. Persons with diabetes are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

Devices have been developed for the automatic monitoring of analyte(s), such as glucose, in bodily fluid such as in the blood stream or in interstitial fluid (“ISF”), or other biological fluid. Some of these analyte measuring devices are configured so that at least a portion of the devices are positioned below a skin surface of a user, e.g., in a blood vessel or in the subcutaneous tissue of a user, so that the monitoring is accomplished in vivo.

With the continued development of analyte monitoring devices and systems, there is a need for such analyte monitoring devices, systems, and methods, as well as for processes for manufacturing analyte monitoring devices and systems that are cost effective, convenient, and provide discreet monitoring to encourage frequent analyte monitoring to improve glycemic control. Additionally, there is a need for such analyte monitoring devices, systems, and methods that reduce pain and trauma associated with analyte monitoring and testing.

While current sensors can be convenient for users, they are also susceptible to malfunctions. These malfunctions can be caused by user error, lack of proper training, poor user coordination, overly complicated procedures, physiological responses to the inserted sensor, and other issues. This can be particularly true for analyte monitoring systems having sensors used to measure an analyte level in ISF, and which are inserted using sharps (also known as “introducers” or “needles”). In addition, some prior art systems may utilize sharps that can create trauma to surrounding tissue at the sensor insertion site, which can lead to inaccurate analyte level measurements. These challenges and others described herein can lead to a failure to properly monitor the patient's analyte level.

Thus, a need exists for more reliable sensor insertion devices, systems and methods, that are easy to use by the patient, less prone to error, and which reduce trauma to an insertion site.

### SUMMARY

According to a first aspect of the present disclosure, there is provided: an insertion apparatus for inserting a sensor into skin of a subject, comprising: a sensor control device comprising a sensor, wherein the sensor is configured to penetrate the skin of the subject; a housing comprising a cover and a device carrier coupled to the cover, wherein the device carrier is configured to releasably retain the sensor control device, and wherein the housing comprises an interface element; and a guide for guiding insertion of the sensor into the skin, wherein the guide is configured to support at least a portion of the sensor, wherein the guide comprises a distal end configured to engage the skin to allow distal movement of the housing relative to the guide, and wherein the guide comprises a retaining element; wherein the insertion apparatus has a first configuration in which the retaining element of the guide is configured to engage with the interface element of the housing to resist distal movement of the housing relative to the guide; and wherein the insertion apparatus is configured to move from the first configuration to a second configuration in response to a distal force on the housing above a threshold force, in which the interface element is configured to disengage from the retaining element to distally move the housing relative to the guide and insert the sensor into the skin.

In some embodiments, the insertion apparatus can be used to position the sensor control device on a human body with the sensor in contact with the wearer's bodily fluid. In particular, the insertion apparatus allows the sensor to be inserted into the skin because the sensor is configured to penetrate the skin of the subject. This penetration is aided by the engagement and disengagement of the retaining element of the guide with the interface element of the housing.

The guide can support sensor insertion and has a retaining element for engaging with an interface element on the housing. This means that, when a user applies a distal force on the housing, the retaining element engages with the interface element to resist the distal force. This is the first configuration of the insertion apparatus. As used herein, the term “distal” is used to preferably mean the direction of sensor insertion towards the skin. Thus, when a user pushes on the housing, pressing the insertion apparatus against the skin, the retaining element resists distal movement of the housing towards the skin. The insertion apparatus is configured to move to the second configuration when the distal force is above a threshold force. The interface element can disengage from the retaining element allowing the housing to distally move relative to the guide. The sensor can then be inserted into the skin. In some embodiments, in the second configuration, the sensor is inserted into the skin.

Due to the sensor penetrating the skin, in some embodiments a sharp (sometimes referred to as a needle) is not required for sensor insertion. Advantages will be apparent to the person skilled in the art. Ease of use may be improved because a sharp may not be required. This can also reduce pain and trauma associated with sharp use. For instance, this may prevent or reduce the risk of an adverse physiological response from sensor insertion. This can also lead to more accurate analyte measurements by reducing trauma to surrounding tissue. Furthermore, design and manufacturing complexity can also be simplified by providing the insertion apparatus without a sharp, and the need to safely dispose of a sharp can be eliminated. By supporting the sensor with the guide, stability of the sensor during insertion can be improved, leading to accurate sensor insertion. Avoiding inclusion of a sharp also means the insertion device is not considered a biohazard.

Other improvements and advantages are provided as well. The purpose and advantages of the present disclosure will be set forth herein and will be apparent from the description that follows.

The threshold force may be predetermined based on the insertion apparatus. For example, the threshold force may be determined by the geometry of the insertion apparatus, in particular of the interface element and the retaining element.

In some embodiments, the retaining element and the interface element are configured to store potential energy whilst resisting distal movement of the housing in response to a distal force on the housing below the threshold force. This allows the insertion apparatus to store potential energy while the user exerts a distal force on the housing without causing distal movement of the housing relative to the guide. In some embodiments, the potential energy can be stored as tension within the retaining element and/or the interface element. By storing energy, this can increase the speed of the sensor for easier insertion.

In some embodiments, the stored potential energy is released when the interface element disengages from the retaining element, accelerating the sensor towards the skin. Under the applied distal force exceeding the threshold, the interface element can overcome the retaining element, releasing the stored potential energy. This can be converted into kinetic energy of the housing as the housing is permitted to move distally relative to the guide. This allows the sensor to move with the housing towards the skin at high speed. Energy can be released to allow the sensor to penetrate the skin. The storing of energy allows a higher speed to be reached by the sensor, and for improved case of use. By requiring a predetermined threshold force, and the feedback to the user through the resistance to distal movement below the threshold, the user can easily apply the desired force for sensor insertion. The momentum from applying to distal force can then be used for sensor insertion without requiring the user to apply the required force from the outset, without requiring the user to freely insert the sensor at the required speed without a guide. This can improve case of use.

In some embodiments, the distal end of the guide is configured to act against the skin in response to the distal force to provide a restoring force for engaging the retaining element with the interface element when the distal force is below the threshold force, and for allowing the housing to distally move relative to the guide when the distal force is above the threshold force. The distal end may be configured to abut the skin, preferably without penetrating the skin. The skin can then provide a restoring force which can be used to resist movement of the interface element and store energy.

In some embodiments, the interface element is arranged on the device carrier. This allows the retaining element to engage with the device carrier for resisting movement of the device carrier.

In some embodiments, the interface element is arranged on the cover. This allows the retaining element to engage with the cover for resisting movement of the device carrier.

In some embodiments, the retaining element comprises a protrusion extending laterally from the guide, and wherein the interface element is arranged proximally of the protrusion in the first configuration and the protrusion is configured to resist distal movement of the interface element. Preferably, the protrusion extends outwards away from the center of the guide. As the interface element is arranged proximally of the protrusion, the protrusion is closer to the skin than the interface element. This means that the interface element engages the protrusion as it moves in the distal direction towards the skin. Preferably, the interface element is arranged to engage the protrusion in response to the distal force on the housing. The size of the protrusion element may affect the threshold force, where a larger protrusion in the lateral direction may require a larger force to overcome.

In some embodiments, the interface element is configured to deflect over the protrusion to move to the second configuration when the distal force is above the threshold force. When the distal force exceeds the threshold, the interface element may deflect over the protrusion, for example by snapping over the protrusion, as the potential energy is released. In other words, the interface element may be deflectable in response to the distal force exceeding the threshold. For example, the interface element may be flexible. In particular, the connection of the interface element to the housing may be flexible to permit deflection of the interface element in response to the distal force. The flexibility of the interface element may affect the threshold force, where a less flexible interface element may require a larger force to deflect.

In some embodiments, the retaining element comprises a recess adjacent to and proximally of the protrusion, wherein the recess is configured to receive the interface element in the first configuration to resist distal movement of the interface element. The recess may have a complementary shape to the interface element. This may improve resistance to distal movement by retaining the interface element within the recess.

In some embodiments, the interface element is configured to deflect out of the recess to move to the second configuration when the distal force is above the threshold force. To overcome the retaining element, the interface element may deflect out of the recess and/or past the protrusion.

In some embodiments, the housing comprises a deflectable arm, and wherein the interface element comprises an end of the deflectable arm. The deflectable arm may define the interface element. The deflectable arm may connect the interface element to the housing. The interface element may be a free end of the deflectable arm, where the other end is connected to the housing. The deflectable arm may be flexible. In some embodiments, the deflectable arm can store potential energy in the form of tension. In particular, under the distal force, the deflectable arm can bend while the interface element is retained by the retaining element. The deflectable arm may be connected to the housing. For instance, the deflectable arm may be connected to the device carrier and/or the cover.

The geometry of the deflectable arm may affect the threshold force, where a less flexible or thicker deflectable arm may require a larger force to deflect and overcome the retaining element. In some embodiments, the geometry of the deflectable arm can be predetermined in order to control the deflection. For example, the thickness, length, and/or orientation angle of the deflectable arm can be defined in order to affect the bending of the deflectable arm in response to the distal force. The material of the deflectable arm can also be used to control the deflection, where more flexible materials can require a smaller force.

In some embodiments, the guide comprises a second retaining element, wherein the second retaining element is arranged distally of the retaining element, and wherein the second retaining element, when in the second configuration, is configured to engage with the interface element to resist distal movement of the housing relative to the guide. The second retaining element may be similar to the (first) retaining element, and it may comprise similar features such as a protrusion and a recess. The second retaining element can be provided to resist distal movement after full sensor insertion. This improves stability once the sensor is inserted. In these or other embodiments, the sensor control device may engage the skin to resist distal movement after sensor insertion, which can improve stability instead of or in combination with the second retaining element.

In some embodiments, an axial distance between the retaining element and the second retaining element corresponds to a distance for movement of the housing for insertion of the sensor into the skin. As used herein, the “axial” distance preferable refers to the distance along the axial direction which includes the distal direction and the proximal direction (being opposite to each other). In other words, the axial direction is preferably along the direction of sensor insertion and withdrawal. The axial distance between the retaining element and the second retaining element is preferably along the length of the guide. Preferably, the defined axial distance means that, when the housing is moved from the first configuration to the second configuration, the interface element can move from engagement with the retaining element into engagement with the second retaining element.

In some embodiments, the guide comprises a third retaining element, wherein the housing comprises a second interface element, and wherein the third retaining element, when in the second configuration, is configured to engage with the second interface element to resist proximal movement of the housing relative to the guide. The second interface element may be separate from the (first) interface element. The third retaining element can resist proximal movement in the second configuration, which retains the insertion apparatus in the locked configuration with the sensor inserted, preventing dislodging of the sensor.

In some embodiments, the housing comprises a second deflectable arm, and wherein the second interface element comprises an end of the second deflectable arm. The second deflectable arm may be similar to the (first) deflectable arm. The second deflectable arm may be connected to the housing. In particular, the second deflectable arm may be connected to the device carrier and/or the cover, which may be the same or different to the part of the housing to which the first deflectable arm is connected. In other words, the housing may comprise a plurality of deflectable arms.

In some embodiments, the insertion apparatus further comprises a biasing element configured to bias the interface element against the guide. The biasing element can push the interface element towards the guide. This makes it more difficult for the interface element to overcome the retaining element, which may increase the potential stored energy. The interface element may be biased against the retaining element, for example being biased to fit within the recess. This can also improve the contact between the interface element and the guide during sensor insertion, which can improve stability. In some examples, the biasing element may be a spring. In other examples, the interface element may be biased against the guide by the deflectable arm. For example, tension within the deflectable arm may push the interface element against the guide.

In some embodiments, the guide comprises a supporting surface arranged distally of the retaining element, and wherein the interface element is configured to contact and slide along the supporting surface when moving to the second configuration. This stabilizes the sensor during insertion. The interface element may be biased against the supporting surface to promote contact, for example by the deflectable arm pushing the interface element against the support surface.

In some embodiments, the supporting surface comprises at least a straight portion parallel to an axial direction. Preferably, the portion is straight in the axial direction, but may be curved in a radial direction perpendicular to the axial direction. For example, the portion may be a cylindrical surface which is straight in the axial direction but curved along the circumference of the guide.

In some embodiments, the supporting surface comprises a ramp portion angled relative to an axial direction and extending laterally outwards. The ramp portion may be arranged adjacent to the straight portion, preferably arranged adjacent to and distally of the straight portion. The ramp portion can act to push the interface element laterally outwards (which may deflect or bend the deflectable arm), slowing the interfaced element and thus the sensor. This can better control the sensor as it is inserted, which may improve stability. This ensures the sensor stays supported by the guide during insertion. This can reduce risk of buckling, bouncing, or deflecting.

In some embodiments, the ramp portion is configured to increase surface contact between the guide and the interface element.

In some embodiments, the ramp portion is arranged at a distal portion of the supporting surface. This allows the sensor to be slowed at the end of the stroke, allowing speed to be built up along the straight portion until control is required near sensor insertion.

In some embodiments, the ramp portion is configured to contact the interface element as the sensor is inserted into the skin. This allows the sensor to be slowed as it approaches the skin, improving control where desired.

In some embodiments, the ramp portion is configured to contact the interface element as the sensor extends beyond the guide when moving into the second configuration. This allows the sensor to be slowed as it approaches the skin, improving control where desired.

In some embodiments, the ramp portion is arranged adjacent to and proximally of the second retaining element. This allows the ramp portion to slow the sensor until distal movement is prevented.

In some embodiments, the guide comprises a second supporting surface arranged proximally of the third retaining element, and wherein the second interface element is configured to contact and slide along the second supporting surface when moving to the second configuration. The second supporting surface may be arranged on the opposite side of the guide to the (first) supporting surface. This allows for greater support of the guide, improving stability of the sensor during insertion.

In some embodiments, the second supporting surface comprises a second ramp portion angled relative to an axial direction and extending laterally inwards. This can be used to slow the sensor similarly to the (first) ramp portion.

In some embodiments, the second ramp portion is configured to increase surface contact between the guide and the second interface element.

In some embodiments, the second ramp portion is arranged at a proximal portion of the second supporting surface. The proximal portion may be further in the proximal direction than the first ramp portion.

In some embodiments, the second ramp portion is configured to contact the second interface element as the sensor is inserted into the skin. This allows the sensor to be slowed as it approaches the skin, improving control where desired.

In some embodiments, the second ramp portion is configured to contact the second interface element as the sensor extends beyond the guide when moving into the second configuration. This allows the sensor to be slowed as it approaches the skin, improving control where desired.

In some embodiments, the second ramp portion is arranged adjacent to and proximally of the third retaining element. This allows the second ramp portion to slow the sensor until the insertion apparatus is locked and proximal movement is prevented.

In some embodiments, the guide comprises a sensor support channel, and wherein the sensor is arranged to distally move through the sensor support channel within the guide in order to guide insertion of the sensor into the skin. The sensor support channel may be arranged as within the guide, for example as a hollow portion. The wall of the hollow portion can then support at least a portion of the sensor. The sensor can then pass through the sensor support channel, supported by the guide.

In some embodiments, at least a portion of the guide is arranged to extend through the device carrier.

In some embodiments, in the second configuration, the sensor control device contacts the skin, and the device carrier is configured to release the sensor control device on the skin. The sensor control device contacting the skin can prevent further distal movement and thus limit sensor insertion. The sensor control device may have an adhesive patch which adheres to the skin, fixing the sensor control device in place.

In some embodiments, the insertion apparatus further comprises a sheath slidably moveable relative to the housing, and wherein the sheath comprises a distal end configured to engage the skin.

In some embodiments, the housing is configured to distally move relative to the sheath in response to a second distal force on the housing.

In some embodiments, distally moving the housing relative to the sheath comprises moving the distal end of the guide into contact with the skin before the movement from the first configuration to the second configuration.

In some embodiments, the sensor comprises a pointed tip configured to penetrate the skin. This can aid penetration by reducing the resistance to sensor insertion.

In some embodiments, the sensor comprises a free length portion configured to provide rigidity to facilitate insertion of the sensor into the skin.

In some embodiments, the sensor is an in vivo analyte sensor configured to measure an analyte level in a bodily fluid of the subject. For example, the analyte may be glucose and the sensor may be a glucose sensor. Other analytes may be used in other embodiments, as disclosed herein.

In some embodiments, the guide comprises a projection element extending from the distal end of the guide, and wherein the projection element is configured to interact with the skin when the distal end engages the skin. This can aid insertion of the sensor. The projection element can protrude from the distal end of the guide and may deform, stretch, and/or pre-pierce the skin to aid sensor insertion.

In some embodiments, the projection element is arranged adjacent to an end of the sensor support channel at the distal end. This allows the sensor exiting the sensor support channel to be guided by the projection element towards the skin.

In some embodiments, the projection element is configured to deform the skin. The projection element can push against the skin to bend the skin to receive the projection element.

In some embodiments, the projection element comprises a pointed tip. This allows the projection element to deform the skin to a point. In other words, this can form an indent with a precise point, to permit precision location of the sensor at the bottom of the indent.

In some embodiments, the projection element comprises a rounded tip. This can tighten the skin without forming a pointed indent, for use in stretching the skin. This can stretch the skin between the end points of the line (or surface). By stretching the skin, the sensor can be more easily inserted.

In some embodiments, the projection element comprises a wall arranged to extend along the distal surface of the guide. By forming a wall (e.g., continuous), the skin can be stretched at multiple points, hold the skin taut.

In some embodiments, the projection element is configured to pre-penetrate the skin to aid insertion of the sensor. This means the projection element can penetrate the skin before the sensor is inserted. This enables easier insertion of the sensor afterwards.

In some embodiments, the projection element comprises a needle. This can be used to produce a small puncture wound with minimal impact on the subject, whilst aiding sensor insertion.

In some embodiments, the needle is an acupuncture needle. This can provide a very small needle with reduced impact on the subject.

According to a second aspect of the present disclosure, there is provided: an insertion apparatus for inserting a sensor into skin of a subject, comprising: a housing comprising a cover and a device carrier coupled to the cover, wherein the device carrier is configured to relcasably retain a sensor control device comprising a sensor, and wherein the housing comprises an interface element; and a guide for guiding insertion of the sensor into the skin, wherein the guide is configured to support at least a portion of the sensor, when the sensor is arranged in the insertion apparatus, wherein the guide comprises a distal end configured to engage the skin to allow distal movement of the housing relative to the guide, and wherein the guide comprises a retaining element; wherein the insertion apparatus has a first configuration in which the retaining element of the guide is configured to engage with the interface element of the housing to resist distal movement of the housing relative to the guide; and wherein the insertion apparatus is configured to move from the first configuration to a second configuration in response to a distal force on the housing above a threshold force, in which the interface element is configured to disengage from the retaining element to distally move the housing relative to the guide for inserting the sensor into the skin.

In this manner, the sensor control device may be separate from the insertion apparatus. In some embodiments, the sensor control device can be loaded into the insertion apparatus, such as described herein.

According to a third aspect of the present disclosure, there is provided: an insertion apparatus for inserting a sensor into skin of a subject, comprising: a sensor control device comprising a sensor, wherein the sensor is configured to penetrate the skin of the subject; a housing; a device carrier coupled to the housing, wherein the device carrier is configured to releasably retain the sensor control device, and wherein the housing and/or the device carrier comprises an interface element; and a guide for guiding insertion of the sensor into the skin, wherein the guide is configured to support at least a portion of the sensor, wherein the guide comprises a distal end configured to engage the skin to allow distal movement of the housing and/or the device carrier relative to the guide, and wherein the guide comprises a retaining element; wherein the insertion apparatus has a first configuration in which the retaining element of the guide is configured to engage with the interface element of the housing and/or the device carrier to resist distal movement of the housing and/or the device carrier relative to the guide; and wherein the insertion apparatus is configured to move from the first configuration to a second configuration in response to a distal force on the housing and/or the device carrier above a threshold force, in which the interface element is configured to disengage from the retaining element to distally move the housing and/or the device carrier relative to the guide and insert the sensor into the skin.

According to a fourth aspect of the present disclosure, there is provided: an insertion apparatus for inserting a sensor into skin of a subject, comprising: a sensor control device comprising a sensor; a housing comprising a cover and a device carrier coupled to the cover, wherein the device carrier is configured to releasably retain the sensor control device; and a guide for guiding insertion of the sensor into the skin, wherein the guide comprises a sensor support channel configured to support at least a portion of the sensor, wherein the guide comprises a distal end configured to engage the skin to allow distal movement of the housing relative to the guide; wherein the guide comprises a projection element extending from the distal end of the guide, and wherein the projection element is configured to interact with the skin when the distal end engages the skin.

In some embodiments, by providing a projection element from the distal end of the guide to interact with the skin, the insertion of the sensor can be aided. For instance, the projection element may protrude from the distal end of the guide. The projection element may extend (e.g., at least) from the distal end of the guide in a direction parallel to the length of the guide (e.g., towards the skin, in use). In some examples, the projection element may be coupled to the distal end of the guide or may be integral with the guide. This may deform the skin to guide insertion, stretch the skin, and/or pre-pierce the skin to aid sensor insertion. Other improvements and advantages are provided as well. The purpose and advantages of the present disclosure will be set forth herein and will be apparent from the description that follows.

In some embodiments, the projection element is arranged adjacent to an end of the sensor support channel at the distal end. This allows the sensor emerging from the sensor support channel to be guided by the projection element towards the skin.

In some embodiments, the projection element is configured to interact with the skin such that the projection element passes through a plane defining a position of the skin. The plane defining the position of the skin corresponds to a position of the upper surface of the skin before the distal end of the guide engages the skin. In other words, the projection element can move the skin out of its previous position. This can form an indent in the skin. Due to the flexibility and elasticity of the skin, when the projection element is removed (when the guide is withdrawn), the skin returns to the default position, such as back to the plane.

In some embodiments, the projection element is configured to deform the skin. The projection element can push against the skin to bend the skin to receive the projection element.

In some embodiments, the projection element is configured to deform the skin to assist aiming of the sensor to aid insertion of the sensor. By deforming the skin to a precise point, the projection element can guide the sensor to a specific location at the bottom of the indent, which can improve the precision of insertion.

In some embodiments, the projection element comprises a pointed tip. This allows the projection element to deform the skin to a point. In other words, this can form an indent with a precise point, to permit precision location of the sensor at the bottom of the indent.

In some embodiments, the pointed tip terminates at a position aligned with an edge of the sensor support channel. This enables the projection element to assist in locating the sensor by guiding the sensor as it exits the sensor support channel.

In some embodiments, the projection element comprises a triangular cross section. This enables a point to be easily manufactured which is strong and stable to deform the skin to a point.

In some embodiments, the projection element is configured to deform the skin to stretch the skin to aid insertion of the sensor. Rather than deforming the skin to a point, the projection element may deform the skin to a line, or even a surface (e.g., a plane). This can stretch the skin between the end points of the line (or surface). By stretching the skin, the sensor can be more easily inserted.

In some embodiments, the projection element comprises a rounded tip. This can tighten the skin without forming a pointed indent, for use in stretching the skin.

In some embodiments, a tip of the projection element is defined by a surface extending in a plane parallel to the distal surface of the guide. This allows the indent to be formed as a line or a surface, tightening the skin from the points along the surface of the tip of the projection element.

In some embodiments, a tip of the projection element is arranged at least at two positions in a plane parallel to the distal surface of the guide. This allows the skin to be stretched between two points.

In some embodiments, the projection element is arranged at least at opposing sides of the sensor support channel. This enables the skin to be stretched over the sensor support channel, where the sensor can be inserted.

In some embodiments, the projection element comprises a wall arranged to extend along the distal surface of the guide. By forming a wall (e.g., continuous), the skin can be stretched at multiple points, hold the skin taut.

In some embodiments, the wall extends around at least part of the edge of the end of the sensor support channel. By arranging the wall close to the sensor support channel, the skin can be tightened near where the sensor can be inserted.

In some embodiments, the wall extends around at least three sides of the end of the sensor support channel. This allows the skin to be tightened over the end of the sensor support channel so the sensor can be inserted through taut skin.

In some embodiments, the projection element is configured to pre-penetrate the skin to aid insertion of the sensor. This means the projection element can penetrate the skin before the sensor is inserted. This enables easier insertion of the sensor afterwards.

In some embodiments, the projection element comprises a needle. This can be used to produce a small puncture wound with minimal impact on the subject, whilst aiding sensor insertion.

In some embodiments, the needle is an acupuncture needle. This can provide a very small needle with reduced impact on the subject.

In some embodiments, the sensor is configured to penetrate the skin. The fourth aspect is particularly advantageous in combination with a sensor configured to penetrate the skin. In other words, in situations where a sharp is not used to insert the sensor, and instead the sensor itself is useful (e.g., using needle-free applicators as described herein), the insertion apparatus is particularly useful to assist insertion of the sensor.

In some embodiments, the sensor comprises a pointed tip configured to penetrate the skin. For example, the sensor can have a sharp tip to assist insertion, which can be beneficial for the insertion apparatus designed to promote sensor insertion.

In some embodiments, the sensor comprises a free length portion configured to provide rigidity to facilitate insertion of the sensor into the skin. This further improves the sensor insertion.

In some embodiments, the insertion apparatus comprises a sharp configured to penetrate the skin. In other examples, the insertion apparatus can be used with other systems including a sharp to penetrate the skin and then a sensor can be inserted. In these situations, the insertion apparatus and the projection element can be used to aid insertion of the sharp and reduce trauma or buckling during insertion.

In some embodiments, the sensor is an in vivo analyte sensor configured to measure an analyte level in a bodily fluid of the subject. For example, the analyte may be glucose and the sensor may be a glucose sensor. Other analytes may be used in other embodiments, as disclosed herein.

According to a fifth aspect of the present disclosure, there is provided: an insertion apparatus for inserting a sensor into skin of a subject, comprising: a housing comprising a cover and a device carrier coupled to the cover, wherein the device carrier is configured to relcasably retain a sensor control device comprising a sensor; and a guide for guiding insertion of the sensor into the skin, wherein the guide comprises a sensor support channel configured to support at least a portion of the sensor when the sensor is arranged in the insertion apparatus, wherein the guide comprises a distal end configured to engage the skin to allow distal movement of the housing relative to the guide; wherein the guide comprises a projection element extending from the distal end of the guide, and wherein the projection element is configured to interact with the skin when the distal end engages the skin.

In this manner, the sensor control device may be separate from the insertion apparatus. In some embodiments, the sensor control device can be loaded into the insertion apparatus, such as described herein.

According to a sixth aspect of the present disclosure, there is provided a guide for guiding insertion of a sensor into skin of a subject, comprising: a sensor support channel configured to support at least a portion of the sensor, wherein the guide comprises a distal end configured to engage the skin; wherein the guide comprises a projection element extending from the distal end of the guide, and wherein the projection element is configured to interact with the skin when the distal end engages the skin.

For example, the guide may be provided independently in isolation of the other features of the insertion apparatus and may be used as part of the insertion apparatus of other aspects. For instance, the guide of the sixth aspect may be provided with features of other aspects, such as features of the housing of the first to fifth aspects. The guide may comprise features described in relation to the fifth aspect, such as the features of the projection element.

Aspects of the present disclosure may be provided in conjunction with each other and features of one aspect may be applied to other aspects. Any feature in one aspect of the present disclosure may be applied to other aspects of the present disclosure, in any appropriate combination. For instance, features of the fourth or fifth aspect may be used in combination with features of the insertion apparatus of the first to third aspects. In particular, the guide of the fourth or fifth aspect may be used with the insertion apparatus of the first to third aspects (such as including features of the retaining elements, the interface elements, and the sheath, for example). It should also be appreciated that particular combinations of the various features described and defined in any aspects of the present disclosure can be implemented and/or supplied and/or used independently.

Other systems, devices, methods, features and advantages of the subject matter described herein will be or will become apparent to one with skill in the art upon examination of the following figures and detailed description. It is intended that all such additional systems, devices, methods, features, and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

Embodiments of the present disclosure are described below, by way of example only, with reference to the accompanying figures.

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of a sensor applicator, reader device, monitoring system, network, and remote system.

FIG. 2A is a block diagram depicting an example embodiment of a reader device.

FIGS. 2B and 2C are block diagrams depicting example embodiments of sensor control devices.

FIGS. 3A to 3G are progressive views of an example embodiment of the assembly and application of the system of FIG. 1 incorporating a two-piece architecture.

FIG. 4A is a side view depicting an example embodiment of an applicator device coupled with a cap.

FIG. 4B is a side perspective view depicting an example embodiment of an applicator device and cap decoupled.

FIG. 4C is a perspective view depicting an example embodiment of a distal end of an applicator device and electronics housing.

FIG. 5 is a proximal perspective view depicting an example embodiment of a tray with sterilization lid coupled.

FIG. 6A is a proximal perspective cutaway view depicting an example embodiment of a tray with sensor delivery components.

FIG. 6B is a proximal perspective view depicting sensor delivery components.

FIG. 7A is side view depicting an example embodiment of a housing.

FIG. 7B is a perspective view depicting an example embodiment of a distal end of a housing.

FIG. 7C is a side cross-sectional view depicting an example embodiment of a housing.

FIG. 8A is a side view depicting an example embodiment of a sheath.

FIG. 8B is a perspective view depicting an example embodiment of a proximal end of a sheath.

FIG. 8C is a close-up perspective view depicting an example embodiment of a distal side of a detent snap of a sheath.

FIG. 8D is a side view depicting an example embodiment of features of a sheath.

FIG. 8E is an end view of an example embodiment of a proximal end of a sheath.

FIG. 9A is a proximal perspective view depicting an example embodiment of a device carrier.

FIG. 9B is a distal perspective view depicting an example embodiment of a device carrier.

FIG. 10 is a proximal perspective view of an example embodiment of a sharp carrier.

FIG. 11 is a side cross-section depicting an example embodiment of a sharp carrier.

FIGS. 12A to 12B are top and bottom perspective views, respectively, depicting an example embodiment of a sensor module.

FIGS. 13A and 13B are perspective and compressed views, respectively, depicting an example embodiment of a sensor connector.

FIG. 14A is a perspective view depicting an example embodiment of a sensor.

FIG. 14B is a side view of an example embodiment of a sensor.

FIG. 14C is a side view of example embodiment of a sensor and a callout view of a tip portion thereof.

FIG. 15A is a perspective view depicting an example embodiment of a sharp module.

FIG. 15B is a perspective view depicting an example embodiment of a sharp module, and a callout view of the distal tip thereof.

FIG. 15C is a close-up side perspective view depicting the distal tip of the sharp module illustrated in FIG. 15B.

FIG. 15D is a cross-sectional view of a sharp embodiment for the sharp module illustrated in FIG. 15B.

FIG. 15E is a close-up perspective view of an example embodiment of a sharp.

FIG. 15F is a close-up side view of an example embodiment of a sharp.

FIG. 15G. is a cross-sectional view of an example embodiment of a sharp.

FIG. 15H is a cross-sectional view of the sharp depicted in FIG. 15G, further comprising an example embodiment of a sensor.

FIG. 15I. is a cross-sectional view of an example embodiment of a sharp.

FIG. 15J is a cross-sectional view of the sharp depicted in FIG. 15I, further comprising an example embodiment of a sensor.

FIG. 15K is a partial perspective view of an example embodiment of a sharp module, and a callout view of the distal tip thereof.

FIG. 15L is a close-up perspective view of the distal tip of an example embodiment of a sharp module comprising a sensor.

FIG. 16A is a cross-sectional view of a sharpless applicator, in accordance with one embodiment of the disclosed subject matter.

FIGS. 16B to 16H are cross-sectional views of the sharpless applicator depicted in FIG. 16A during various stages of operation.

FIG. 16I is a cross-sectional view depicting an example embodiment of a sharpless applicator illustrated in FIG. 16A.

FIGS. 16J and 16J-1 are a cross-sectional view and a call-out close-up view, respectively, depicting an example embodiment of a guide-sensor control device assembly.

FIGS. 17A, 17A-1, and 17A-2 are a cross-sectional view of an example embodiment of a sharpless applicator and callout views thereof, in accordance with one embodiment of the disclosed subject matter.

FIGS. 17B to 17D are cross-sectional views of the sharpless applicator depicted in FIG. 17A during various stages of operation.

FIGS. 18A to 18C are cross-sectional views of a powered sharpless applicator during various stages of operation.

FIG. 19A is a cross-sectional view of an insertion apparatus, in accordance with a first embodiment of the disclosure, in a first configuration.

FIG. 19B is a close-up cross-sectional view of the insertion apparatus of FIG. 19A.

FIG. 20A is a cross-sectional view of the insertion apparatus of FIG. 19A, between the first configuration and a second configuration.

FIG. 20B is a close-up cross-sectional view of the insertion apparatus of FIG. 20A.

FIG. 21A is a cross-sectional view of the insertion apparatus of FIG. 19A, in the second configuration.

FIG. 21B is a close-up cross-sectional view of the insertion apparatus of FIG. 21A.

FIG. 22A is a perspective view of a guide for an insertion apparatus, in accordance with a second embodiment of the disclosure.

FIG. 22B is a close-up perspective view of the guide of FIG. 22A.

FIG. 23 is a cross-sectional view of an insertion apparatus, in accordance with a third embodiment of the disclosure, in a first configuration.

FIG. 24A is a cross-sectional view of the insertion apparatus of FIG. 23, in a second configuration.

FIG. 24B is a close-up cross-sectional view of the insertion apparatus of FIG. 24A.

FIG. 25A is a cross-sectional view of the insertion apparatus of FIG. 23, between the second configuration and a third configuration.

FIG. 25B is a close-up cross-sectional view of the insertion apparatus of FIG. 25A.

FIG. 26A is a cross-sectional view of the insertion apparatus of FIG. 23, in the third configuration.

FIG. 26B is a close-up cross-sectional view of the insertion apparatus of FIG. 26A.

FIG. 27A is a close-up perspective view of a guide for an insertion apparatus, in accordance with a fourth embodiment of the disclosure.

FIG. 27B is a close-up side perspective view of the guide of FIG. 27A.

FIG. 27C is a close-up side perspective view of the guide of FIG. 27B.

FIG. 28A is a close-up perspective view of a guide for an insertion apparatus, in accordance with a fifth embodiment of the disclosure.

FIG. 28B is a close-up side perspective view of the guide of FIG. 28A.

FIG. 28C is a close-up side perspective view of the guide of FIG. 28B.

FIG. 29A is a close-up perspective view of a guide for an insertion apparatus, in accordance with a sixth embodiment of the disclosure.

FIG. 29B is a close-up side perspective view of the guide of FIG. 29A.

FIG. 29C is a close-up side perspective view of the guide of FIG. 29B.

FIG. 30 is a close-up perspective view of a guide for an insertion apparatus, in accordance with a seventh embodiment of the disclosure.

### DETAILED DESCRIPTION

Before the present disclosure is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include systems, devices, and methods for the use of analyte sensor insertion applicators for use with in vivo analyte monitoring systems. An applicator can be provided to the user in a sterile package with an electronics housing of the sensor control device contained therein. According to some embodiments, a structure separate from the applicator, such as a container, can also be provided to the user as a sterile package with a sensor module and a sharp module contained therein. The user can couple the sensor module to the electronics housing, and can couple the sharp to the applicator with an assembly process that involves the insertion of the applicator into the container in a specified manner. In other embodiments, the applicator, sensor control device, sensor module, and sharp module can be provided in a single package. The applicator can be used to position the sensor control device on a human body with a sensor in contact with the wearer's bodily fluid. The embodiments provided herein are improvements to reduce the likelihood that a sensor is improperly inserted or damaged, or elicits an adverse physiological response. Other improvements and advantages are provided as well. The various configurations of these devices are described in detail by way of the embodiments which are only examples.

Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed, and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

As mentioned, a number of embodiments of systems, devices, and methods are described herein that provide for improved sensor insertion devices for use with in vivo analyte monitoring systems. Many embodiments of the present disclosure are designed to: improve the method of sensor insertion with respect to in vivo analyte monitoring systems, minimize trauma to an insertion site during a sensor insertion process, and reduce overall interference with sensor performance. Some embodiments, for example, include an insertion apparatus having a guide with a retaining element for engaging with an interface element on the housing to resist distal movement of the housing relative to the guide. This can be used to store potential energy during the resisting distal movement, and release the energy to accelerate the sensor towards the skin. This can improve sensor insertion, and can allow the insertion apparatus to be provided without a needle. This can allow for a smaller skin penetration and, thus, create a smaller wound with less trauma at the insertion site, which can reduce the chance of early signal attenuation (“ESA”). Overall, these embodiments can improve the likelihood of a successful sensor insertion and reduce the amount of trauma at the insertion site, to name a few advantages.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Exemplary In Vivo Analyte Monitoring System

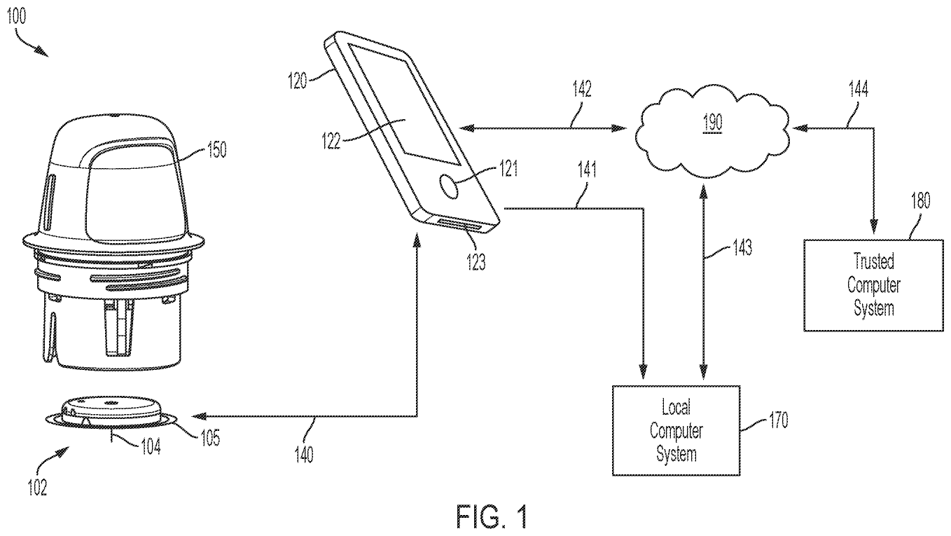

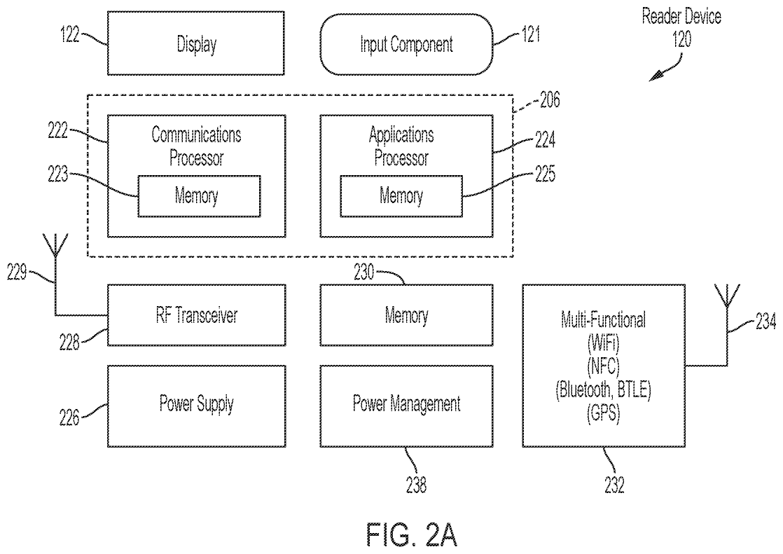

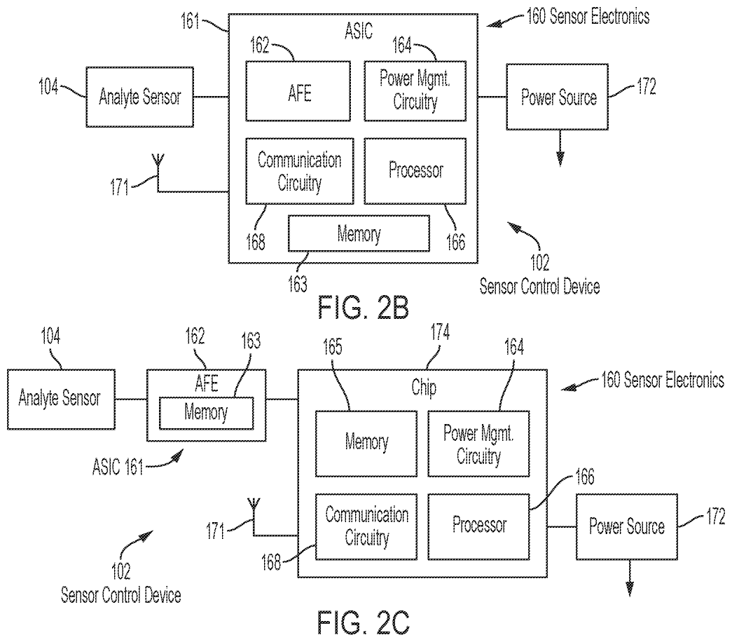

FIG. 1 is a conceptual diagram depicting an example embodiment of an analyte monitoring system 100 that includes a sensor applicator 150, a sensor control device 102, and a reader device 120. Sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can monitor applications installed in memory on reader device 120 using screen 122 and input 121 and the device battery can be recharged using power port 123. More detail about reader device 120 is set forth with respect to FIG. 2A below. Reader device 120 can communicate with local computer system 170 via a communication path 141 using a wired or wireless technique. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy, Wi-Fi or others. Local computer system 170 can communicate via communications path 143 with a network 190 similar to how reader device 120 can communicate via a communications path 142 with network 190, by wired or wireless technique as described previously. Network 190 can be any of a number of networks, such as private networks and public networks, local area or wide area networks, and so forth. A trusted computer system 180 can include a server and can provide authentication services and secured data storage and can communicate via communications path 144 with network 190 by wired or wireless technique.

### Exemplary Reader Device

FIG. 2A is a block diagram depicting an example embodiment of a reader device configured as a smartphone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further included can be a multi-functional transceiver 232 which can communicate over Wi-Fi, NFC, Bluetooth, BTLE, and GPS with an antenna 234. As understood by one of skill in the art, these components are electrically and communicatively coupled in a manner to make a functional device.

### Exemplary Sensor Control Devices

FIGS. 2B and 2C are block diagrams depicting example embodiments of sensor control device 102 having analyte sensor 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Exemplary Assembly Processes for Sensor Control Devices

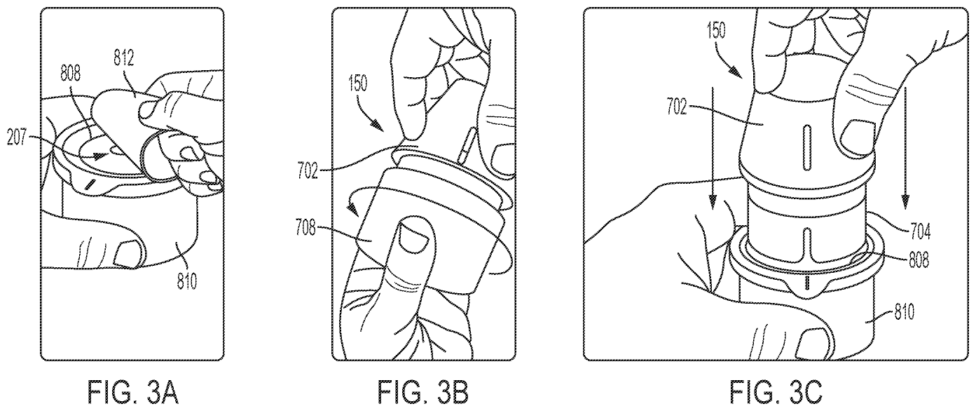

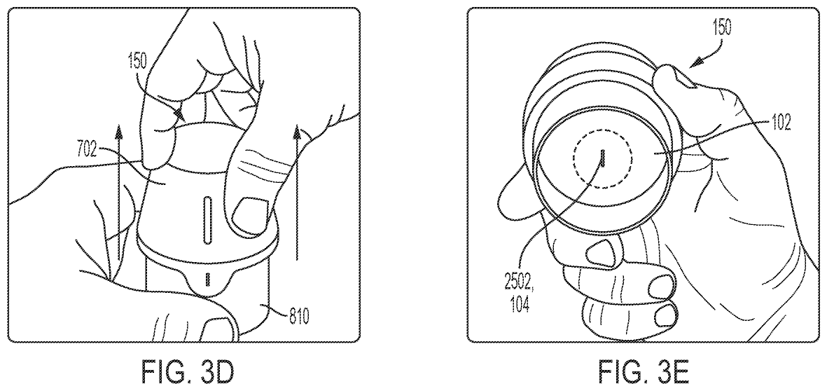

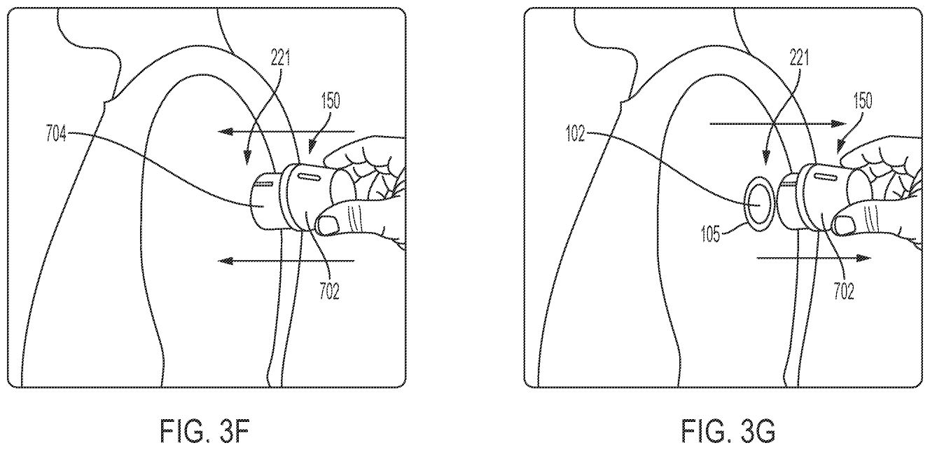

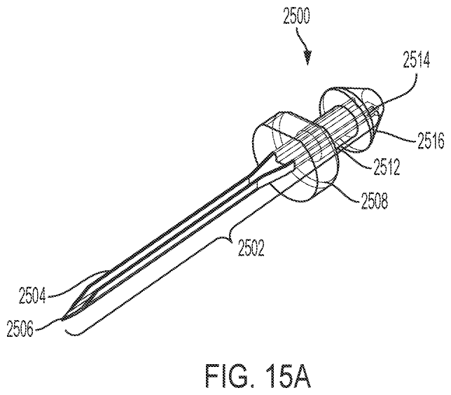

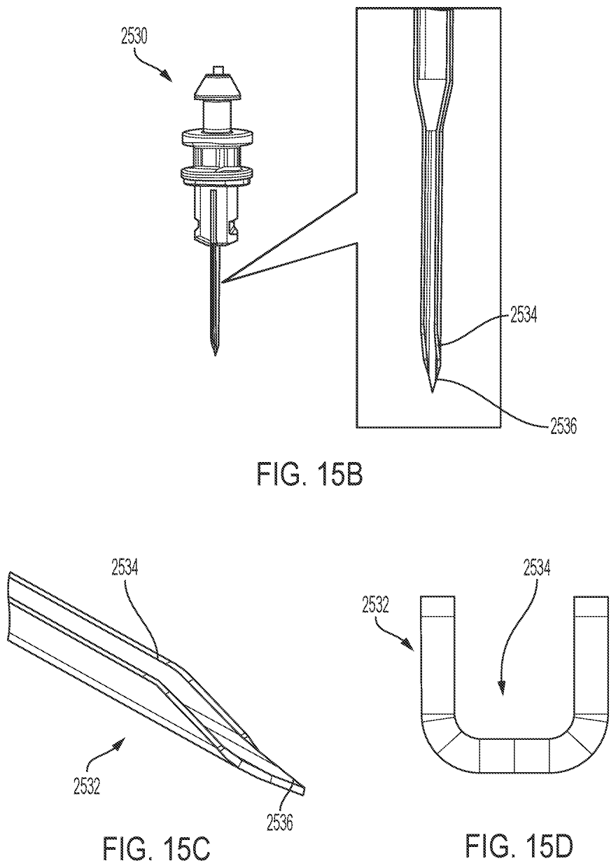

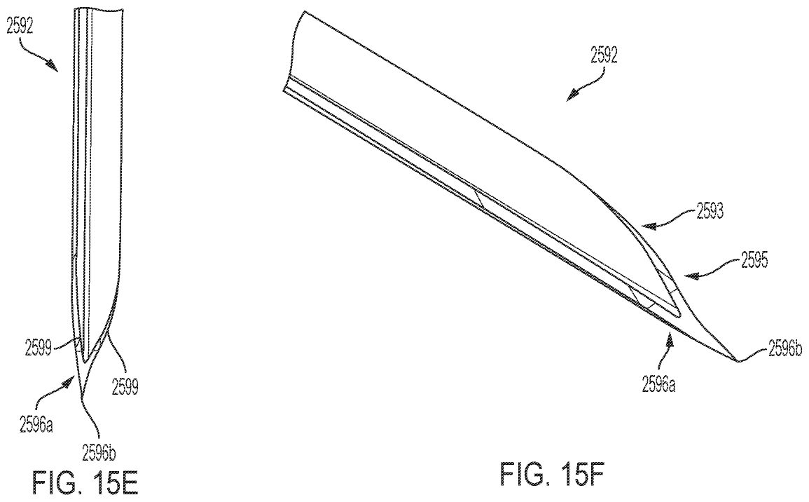

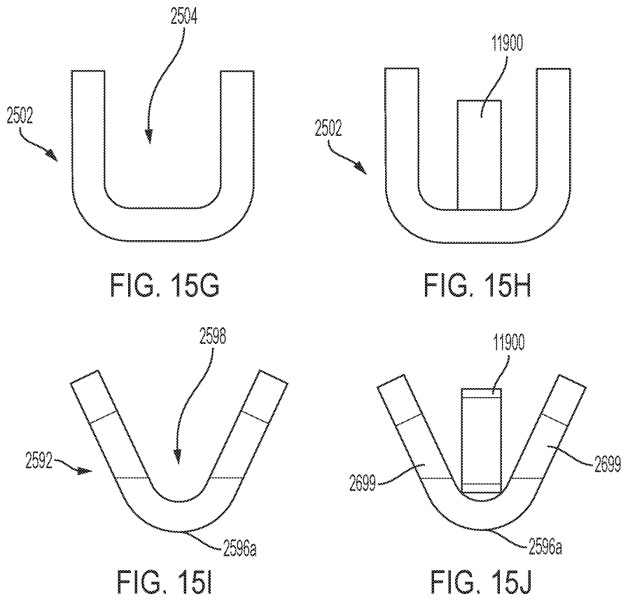

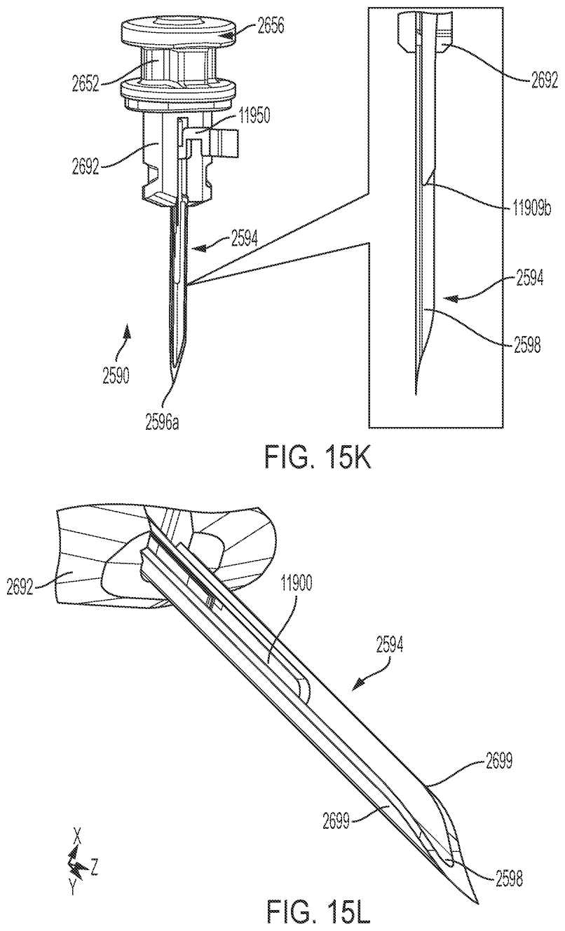

According to some embodiments, the components of sensor control device 102 can be acquired by a user in multiple packages requiring final assembly by the user before delivery to an appropriate user location. FIGS. 3A-3E depict an example embodiment of an assembly process for sensor control device 102 by a user, including preparation of separate components before coupling the components in order to ready the sensor for delivery. In other embodiments, such as those described with respect to FIGS. 15B to 15K, components of the sensor control device 102 and applicator 150 can be acquired by a user in a single package. FIGS. 3F-3G depict an example embodiment of delivery of sensor control device 102 to an appropriate user location by selecting the appropriate delivery location and applying device 102 to the location.

FIG. 3A depicts a sensor container or tray 810 that has a removable lid 812. The user prepares the sensor tray 810 by removing the lid 812, which acts as a sterile barrier to protect the internal contents of the sensor tray 810 and otherwise maintain a sterile internal environment. Removing the lid 812 exposes a platform 808 positioned within the sensor tray 810, and a plug assembly 207 (partially visible) is arranged within and otherwise strategically embedded within the platform 808. The plug assembly 207 includes a sensor module (not shown) and a sharp module (not shown). The sensor module carries the sensor 104 (FIG. 1), and the sharp module carries an associated sharp used to help deliver the sensor 104 transcutaneously under the user's skin during application of the sensor control device 102 (FIG. 1).

FIG. 3B depicts the sensor applicator 150 and the user preparing the sensor applicator 150 for final assembly. The sensor applicator 150 includes a housing 702 sealed at one end with an applicator cap 708. In some embodiments, for example, an O-ring or another type of sealing gasket may seal an interface between the housing 702 and the applicator cap 708. In at least one embodiment, the O-ring or sealing gasket may be molded onto one of the housing 702 and the applicator cap 708. The applicator cap 708 provides a barrier that protects the internal contents of the sensor applicator 150. In particular, the sensor applicator 150 contains an electronics housing (not shown) that retains the electrical components for the sensor control device 102 (FIG. 1), and the applicator cap 708 may or may not maintain a sterile environment for the electrical components. Preparation of the sensor applicator 150 includes uncoupling the housing 702 from the applicator cap 708, which can be accomplished by unscrewing the applicator cap 708 from the housing 702. The applicator cap 708 can then be discarded or otherwise placed aside.

FIG. 3C depicts the user inserting the sensor applicator 150 into the sensor tray 810. The sensor applicator 150 includes a sheath 704 configured to be received by the platform 808 to temporarily unlock the sheath 704 relative to the housing 702, and also temporarily unlock the platform 808 relative to the sensor tray 810. Advancing the housing 702 into the sensor tray 810 results in the plug assembly 207 (FIG. 3A) arranged within the sensor tray 810, including the sensor and sharp modules, being coupled to the electronics housing arranged within the sensor applicator 150.

In FIG. 3D, the user removes the sensor applicator 150 from the sensor tray 810 by proximally retracting the housing 702 with respect to the sensor tray 810.

FIG. 3E depicts the bottom or interior of the sensor applicator 150 following removal from the sensor tray 810 (FIGS. 3A and 3C). The sensor applicator 150 is removed from the sensor tray 810 with the sensor control device 102 fully assembled therein and positioned for delivery to the target monitoring location. As illustrated, a sharp 2502 extends from the bottom of the sensor control device 102 and carries a portion of the sensor 104 within a hollow or recessed portion thereof. The sharp 2502 is configured to penetrate the skin of a user and thereby place the sensor 104 into contact with bodily fluid.

FIGS. 3F and 3G depict example delivery of the sensor control device 102 to a target monitoring location 221, such as the back of an arm of the user. FIG. 3F shows the user advancing the sensor applicator 150 toward the target monitoring location 221. Upon engaging the skin at the target monitoring location 221, the sheath 704 collapses into the housing 702, which allows the sensor control device 102 (FIGS. 3E and 3G) to advance into engagement with the skin. With the help of the sharp 2502 (FIG. 3E), the sensor 104 (FIG. 3E) is advanced transcutaneously into the patient's skin at the target monitoring location 221.

FIG. 3G shows the user retracting the sensor applicator 150 from the target monitoring location 221, with the sensor control device 102 successfully attached to the user's skin. The adhesive patch 105 (FIG. 1) applied to the bottom of sensor control device 102 adheres to the skin to secure the sensor control device 102 in place. The sharp 2502 (FIG. 3E) is automatically retracted when the housing 702 is fully advanced at the target monitoring location 221, while the sensor 104 (FIG. 3E) is left in position to measure analyte levels.

According to some embodiments, system 100, as described with respect to FIGS. 3A-3G and elsewhere herein, can provide a reduced or eliminated chance of accidental breakage, permanent deformation, or incorrect assembly of applicator components compared to prior art systems. Since applicator housing 702 directly engages platform 808 while sheath 704 unlocks, rather than indirect engagement via sheath 704, relative angularity between sheath 704 and housing 702 will not result in breakage or permanent deformation of the arms or other components. The potential for relatively high forces (such as in conventional devices) during assembly will be reduced, which in turn reduces the chance of unsuccessful user assembly. Further details regarding embodiments of applicators, their components, and variants thereof, are described in U.S. Patent Publication Nos. 2013/0150691, 2016/0331283, and 2018/0235520, all of which are incorporated by reference herein in their entireties and for all purposes.

### Example Embodiment of Sensor Applicator Device

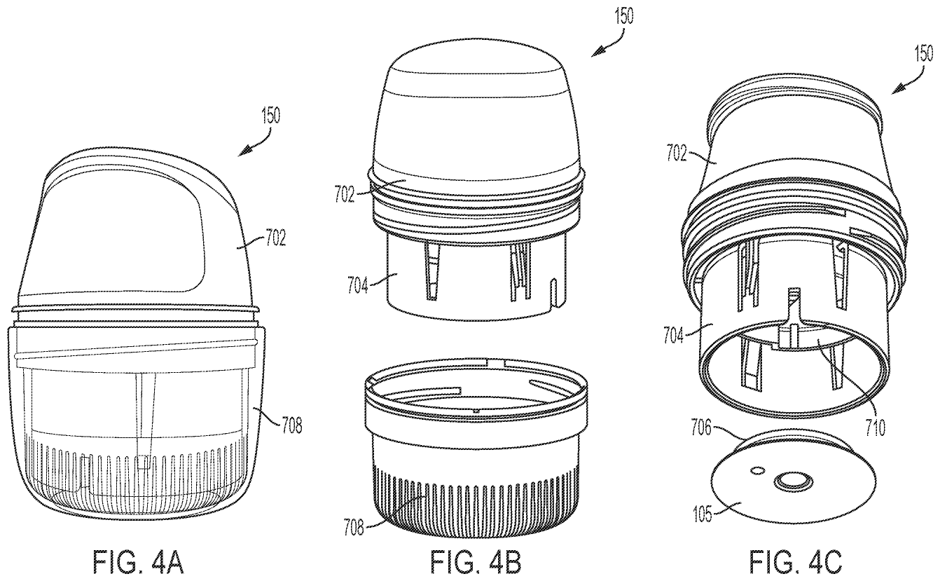

FIG. 4A is a side view depicting an example embodiment of an applicator device 150 coupled with screw cap 708. This is one example of how applicator 150 is shipped to and received by a user, prior to assembly by the user with a sensor. In other embodiments, applicator 150 can be shipped to the user with the sensor and sharp contained therein. FIG. 4B is a side perspective view depicting applicator 150 and cap 708 after being decoupled. FIG. 4C is a perspective view depicting an example embodiment of a distal end of an applicator device 150 with electronics housing 706 and adhesive patch 105 removed from the position they would have retained within device carrier 710 of sheath 704, when cap 708 is in place.

### Exemplary Tray and Sensor Module Assembly

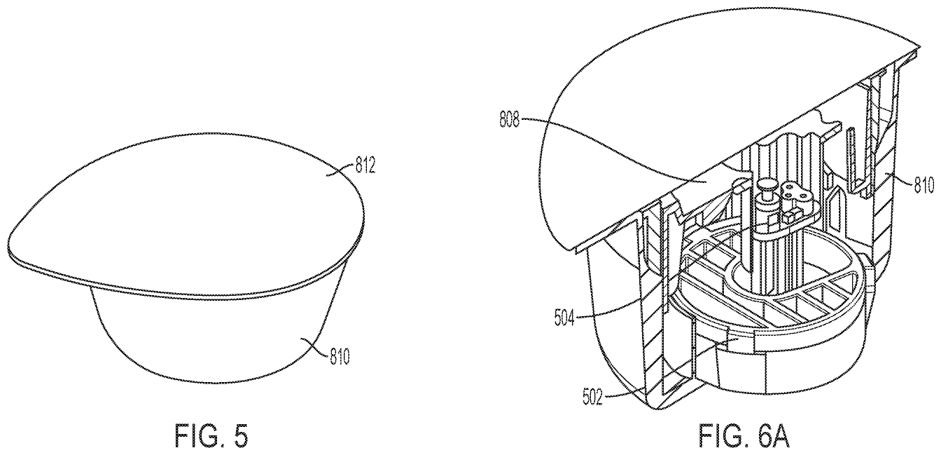

FIG. 5 is a proximal perspective view depicting an example embodiment of a tray 810 with sterilization lid 812 removably coupled thereto, which may be representative of how the package is shipped to and received by a user prior to assembly.

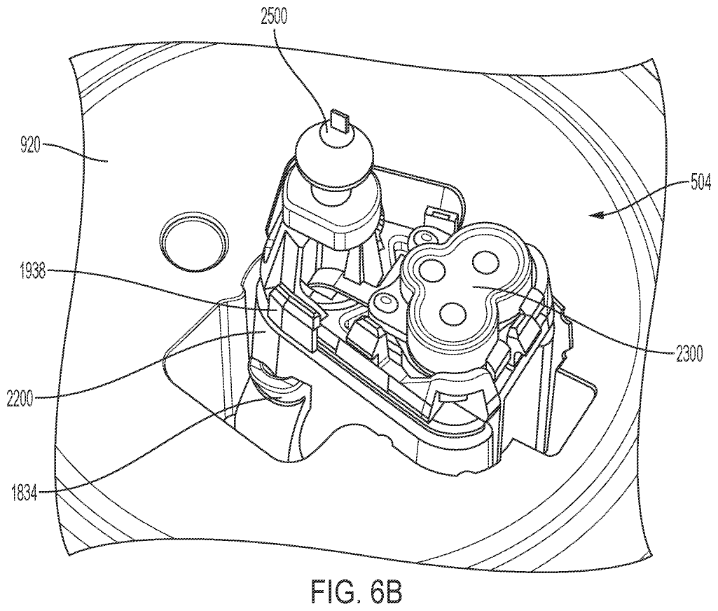

FIG. 6A is a proximal perspective cutaway view depicting sensor delivery components within tray 810. Platform 808 is slidably coupled within tray 810. Desiccant 502 is stationary with respect to tray 810. Sensor module 504 is mounted within tray 810.

FIG. 6B is a proximal perspective view depicting sensor module 504 in greater detail. Here, retention arm extensions 1834 of platform 808 releasably secure sensor module 504 in position. Module 2200 is coupled with connector 2300, sharp module 2500 and sensor (not shown) such that during assembly they can be removed together as sensor module 504.

### Example Embodiment of Applicator Housing

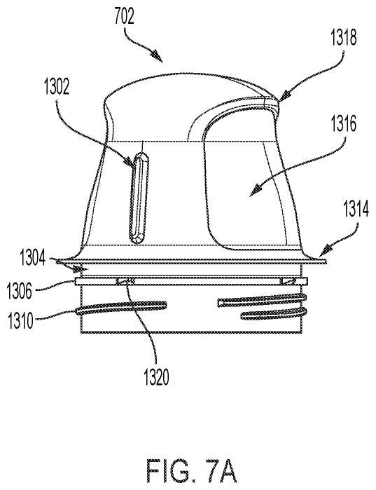

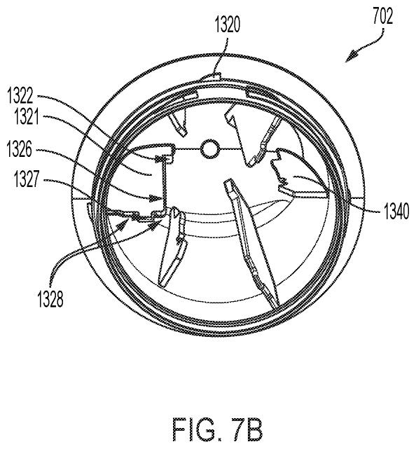

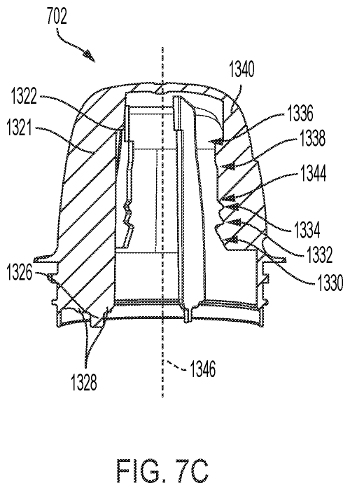

FIG. 7A is side view depicting an example embodiment of the applicator housing 702 that can include an internal cavity with support structures for applicator function. A user can push housing 702 in a distal direction to activate the applicator assembly process and then also to cause delivery of sensor control device 102, after which the cavity of housing 702 can act as a receptacle for a sharp. In the example embodiment, various features are shown including housing orienting feature 1302 for orienting the device during assembly and use. Tamper ring groove 1304 can be a recess located around an outer circumference of housing 702, distal to a tamper ring protector 1314 and proximal to a tamper ring retainer 1306. Tamper ring groove 1304 can retain a tamper ring so users can identify whether the device has been tampered with or otherwise used. Housing threads 1310 can secure housing 702 to complimentary threads on cap 708 (FIGS. 4A and 4B) by aligning with complimentary cap threads and rotating in a clockwise or counterclockwise direction. A side grip zone 1316 of housing 702 can provide an exterior surface location where a user can grip housing 702 in order to use it. Grip overhang 1318 is a slightly raised ridge with respect to side grip zone 1316 which can aid in case of removal of housing 702 from cap 708. A shark tooth 1320 can be a raised section with a flat side located on a clockwise edge to shear off a tamper ring (not shown), and hold tamper ring in place after a user has unscrewed cap 708 and housing 702. In the example embodiment four shark teeth 1320 are used, although more or less can be used as desired.

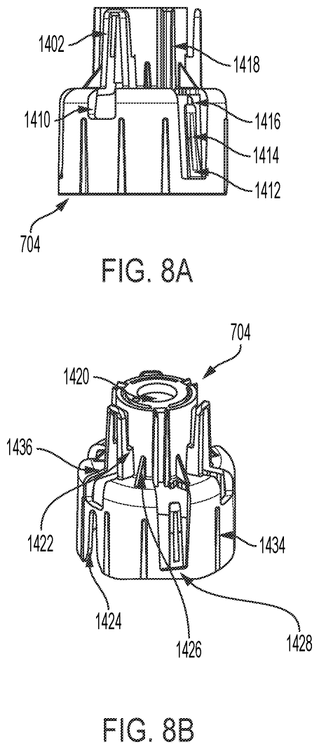

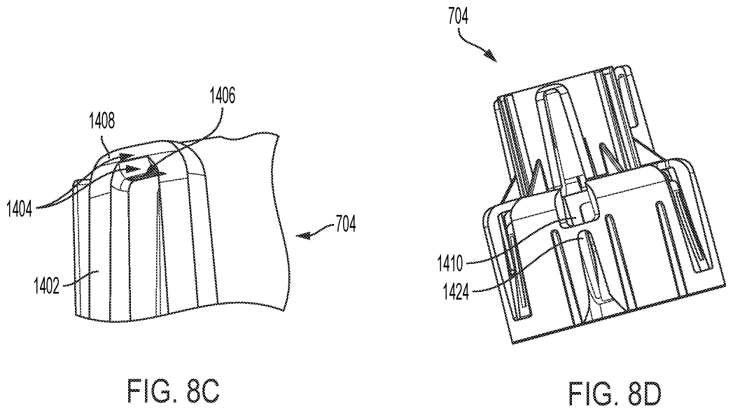

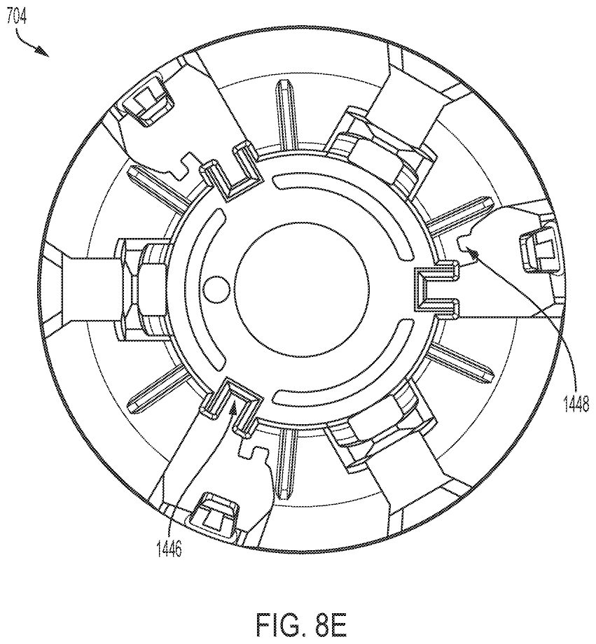

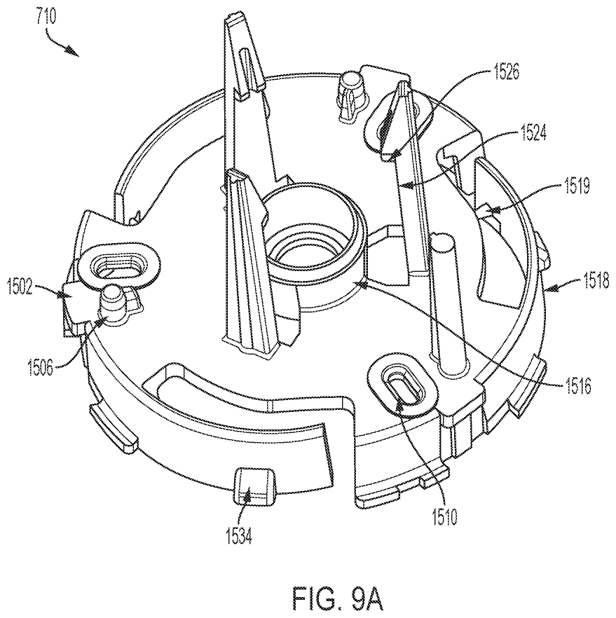

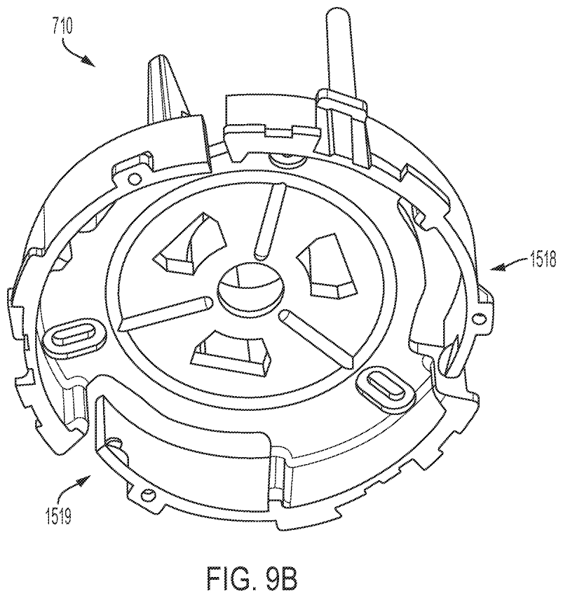

FIG. 7B is a perspective view depicting a distal end of housing 702. Here, three housing guide structures (or “guide ribs”) 1321 are located at 120 degree angles with respect to each other, and at 60 degree angles with respect to locking structures (or “locking ribs”) 1340, of which there are also three at 120 degree angles with respect to each other. Other angular orientations, either symmetric or asymmetric, can be used, as well as any number of one or more structures 1321 and 1340. Here, each structure 1321 and 1340 is configured as a planar rib, although other shapes can be used. Each guide rib 1321 includes a guide edge (also called a “sheath guide rail”) 1326 that can pass along a surface of sheath 704 (e.g., guide rail 1418 described with respect to FIG. 8A). An insertion hard stop 1322 can be a flat, distally facing surface of housing guide rib 1321 located near a proximal end of housing guide rib 1321. Insertion hard stop 1322 provides a surface for a device carrier travel limiter face 1420 of a sheath 704 (FIG. 8B) to abut during use, preventing device carrier travel limiter face 1420 from moving any further in a proximal direction. A carrier interface post 1327 passes through an aperture 1510 (FIG. 9A) of device carrier 710 during an assembly. A device carrier interface 1328 can be a rounded, distally facing surface of housing guide ribs 1321 which interfaces with device carrier 710.

FIG. 7C is a side cross-section depicting an example embodiment of a housing. In the example embodiment, side cross-sectional profiles of housing guide rib 1321 and locking rib 1340 are shown. Locking rib 1340 includes sheath snap lead-in feature 1330 near a distal end of locking rib 1340 which flares outward from central axis 1346 of housing 702 distally. Each sheath snap lead-in feature 1330 causes detent snap round 1404 of detent snap 1402 of sheath 704 as shown in FIG. 8C to bend inward toward central axis 1346 as sheath 704 moves towards the proximal end of housing 702. Once past a distal point of sheath snap lead-in feature 1330, detent snap 1402 of sheath 704 is locked into place in locked groove 1332. As such, detent snap 1402 cannot be easily moved in a distal direction due to a surface with a near perpendicular plane to central axis 1346, shown as detent snap flat 1406 in FIG. 8C.

As housing 702 moves further in a proximal direction toward the skin surface, and as sheath 704 advances toward the distal end of housing 702, detent snaps 1402 shift into the unlocked grooves 1334, and applicator 150 is in an “armed” position, ready for use. When the user further applies force to the proximal end of housing 702, while sheath 704 is pressed against the skin, detent snap 1402 passes over firing detent 1344. This begins a firing sequence due to release of stored energy in the deflected detent snaps 1402, which travel in a proximal direction relative to the skin surface, toward sheath stopping ramp 1338 which is slightly flared outward with respect to central axis 1346 and slows sheath 704 movement during the firing sequence. The next groove encountered by detent snap 1402 after unlocked groove 1334 is final lockout groove 1336 which detent snap 1402 enters at the end of the stroke or pushing sequence performed by the user. Final lockout recess 1336 can be a proximally-facing surface that is perpendicular to central axis 1346 which, after detent snap 1402 passes, engages a detent snap flat 1406 and prevents reuse of the device by securely holding sheath 704 in place with respect to housing 702. Insertion hard stop 1322 of housing guide rib 1321 prevents sheath 704 from advancing proximally with respect to housing 702 by engaging device carrier travel limiter face 1420.

### Example Embodiment of Applicator Sheath

FIGS. 8A and 8B are a side view and perspective view, respectively, depicting an example embodiment of sheath 704. In this example embodiment, sheath 704 can stage sensor control device 102 above a user's skin surface prior to application. Sheath 704 can also contain features that help retain a sharp in a position for proper application of a sensor, determine the force required for sensor application, and guide sheath 704 relative to housing 702 during application. Detent snaps 1402 are near a proximal end of sheath 704, described further with respect to FIG. 8C below. Sheath 704 can have a generally cylindrical cross section with a first radius in a proximal section (closer to top of figure) that is shorter than a second radius in a distal section (closer to bottom of figure). Also shown are a plurality of detent clearances 1410, three in the example embodiment. Sheath 704 can include one or more detent clearances 1410, each of which can be a cutout with room for sheath snap lead-in feature 1330 to pass distally into until a distal surface of locking rib 1340 contacts a proximal surface of detent clearance 1410.

Guide rails 1418 are disposed between device carrier traveler limiter face 1420 at a proximal end of sheath 704 and a cutout around lock arms 1412. Each guide rail 1418 can be a channel between two ridges where the guide edge 1326 of housing guide rib 1321 can slide distally with respect to sheath 704.

Lock arms 1412 are disposed near a distal end of sheath 704 and can include an attached distal end and a free proximal end, which can include lock arm interface 1416. Lock arms 1412 can lock device carrier 710 to sheath 704 when lock arm interface 1416 of lock arms 1412 engage lock interface 1502 of device carrier 710. Lock arm strengthening ribs 1414 can be disposed near a central location of each lock arm 1412 and can act as a strengthening point for an otherwise weak point of each lock arm 1412 to prevent lock arm 1412 from bending excessively or breaking.

Detent snap stiffening features 1422 can be located along the distal section of detent snaps 1402 and can provide reinforcement to detent snaps 1402. Alignment notch 1424 can be a cutout near the distal end of sheath 704, which provides an opening for user alignment with sheath orientation feature of platform 808. Stiffening ribs 1426 can include buttresses, that are triangularly shaped here, which provide support for detent base 1436. Housing guide rail clearance 1428 can be a cutout for a distal surface of housing guide rib 1321 to slide during use.

FIG. 8C is a close-up perspective view depicting an example embodiment of detent snap 1402 of sheath 704. Detent snap 1402 can include a detent snap bridge 1408 located near or at its proximal end. Detent snap 1402 can also include a detent snap flat 1406 on a distal side of detent snap bridge 1408. An outer surface of detent snap bridge 1408 can include detent snap rounds 1404 which are rounded surfaces that allow for easier movement of detent snap bridge 1408 across interior surfaces of housing 702 such as, for example, locking rib 1340.

FIG. 8D is a side view depicting an example embodiment of sheath 704. Here, alignment notch 1424 can be relatively close to detent clearance 1410. Detent clearance 1410 is in a relatively proximal location on distal portion of sheath 704.

FIG. 8E is an end view depicting an example embodiment of a proximal end of sheath 704. Here, a back wall for guide rails 1446 can provide a channel to slidably couple with housing guide rib 1321 of housing 702. Sheath rotation limiter 1448 can be notches which reduce or prevent rotation of the sheath 704. FIG. 8F is a perspective view depicting an example embodiment of a compressible distal end 1450, which can be attached and/or detached from a sheath 704 of an applicator 150. In a general sense, the embodiments described herein operate by flattening and stretching a skin surface at a predetermined site for sensor insertion. Moreover, the embodiments described herein may also be utilized for other medical applications, such as, e.g., transdermal drug delivery, needle injection, wound closure stitches, device implantation, the application of an adhesive surface to the skin, and other like applications.

By way of background, those of skill the art will appreciate that skin is a highly anisotropic tissue from a biomechanical standpoint and varies largely between individuals. This can affect the degree to which communication between the underlying tissue and the surrounding environment can be performed, e.g., with respect to drug diffusion rates, the ability to penetrate skin with a sharp, or sensor insertion into the body at a sharp-guided insertion site.

### Example Embodiments of Device Carriers

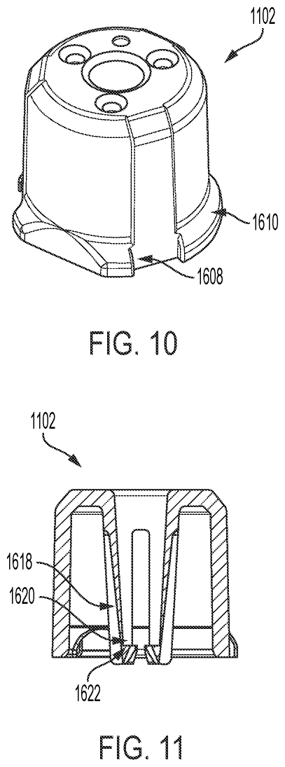

FIG. 9A is a proximal perspective view depicting an example embodiment of device carrier 710 that can retain sensor electronics within applicator 150. It can also retain sharp carrier 1102 with sharp module 2500. In this example embodiment, device carrier 710 generally has a hollow round flat cylindrical shape, and can include one or more deflectable sharp carrier lock arms 1524 (e.g., three) extending proximally from a proximal surface surrounding a centrally located spring alignment ridge 1516 for maintaining alignment of spring 1104. Each lock arm 1524 has a detent or retention feature 1526 located at or near its proximal end. Shock lock 1534 can be a tab located on an outer circumference of device carrier 710 extending outward and can lock device carrier 710 for added safety prior to firing. Rotation limiter 1506 can be a proximally extending relatively short protrusion on a proximal surface of device carrier 710 which limits rotation of carrier 710. Sharp carrier lock arms 1524 can interface with sharp carrier 1102 as described with reference to FIGS. 10A and 10B below.

FIG. 9B is a distal perspective view of device carrier 710. Here, one or more sensor electronics retention spring arms 1518 (e.g., three) are normally biased towards the position shown and include a detent 1519 that can pass over the distal surface of electronics housing 706 of device 102 when housed within recess or cavity 1521. In certain embodiments, after sensor control device 102 has been adhered to the skin with applicator 150, the user pulls applicator 150 in a proximal direction, i.e., away from the skin. The adhesive force retains sensor control device 102 on the skin and overcomes the lateral force applied by spring arms 1518. As a result, spring arms 1518 deflect radially outwardly and disengage detents 1519 from sensor control device 102 thereby releasing sensor control device 102 from applicator 150.

### Example Embodiments of Sharp Carriers

FIGS. 10 and 11 are a proximal perspective view and a side cross-sectional view, respectively, depicting an example embodiment of sharp carrier 1102. Sharp carrier 1102 can grasp and retain sharp module 2500 within applicator 150. Near a distal end of sharp carrier 1102 can be anti-rotation slots 1608 which prevent sharp carrier 1102 from rotating when located within a central area of sharp carrier lock arms 1524 (as shown in FIG. 9A). Anti-rotation slots 1608 can be located between sections of sharp carrier base chamfer 1610, which can ensure full retraction of sharp carrier 1102 through sheath 704 upon retraction of sharp carrier 1102 at the end of the deployment procedure.

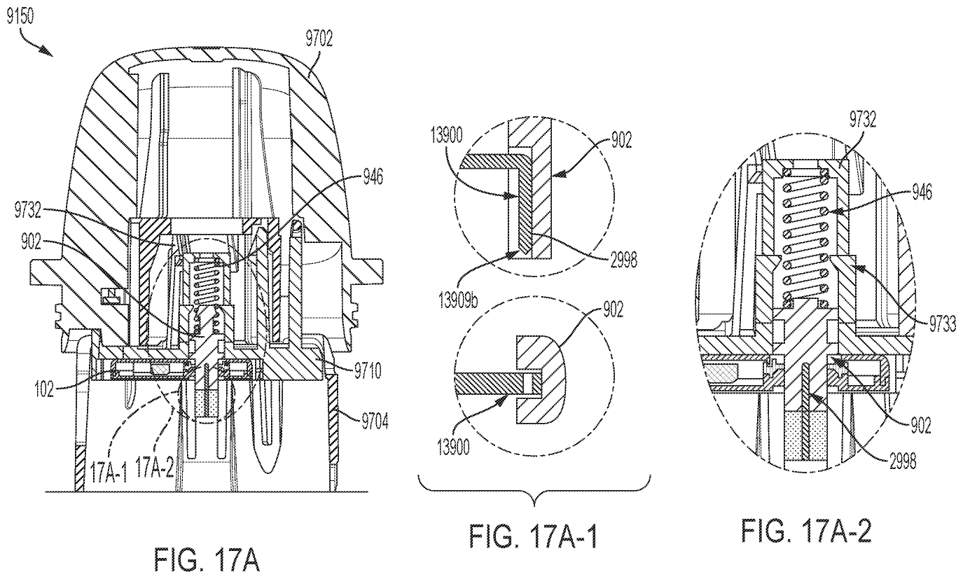

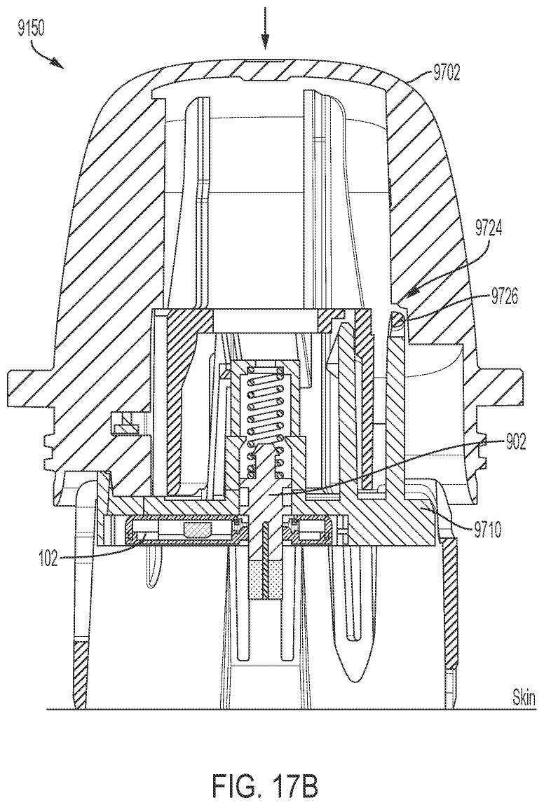

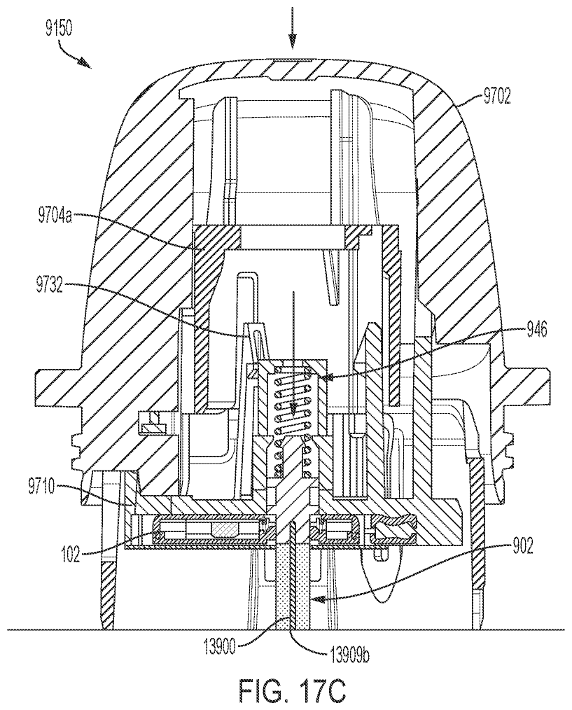

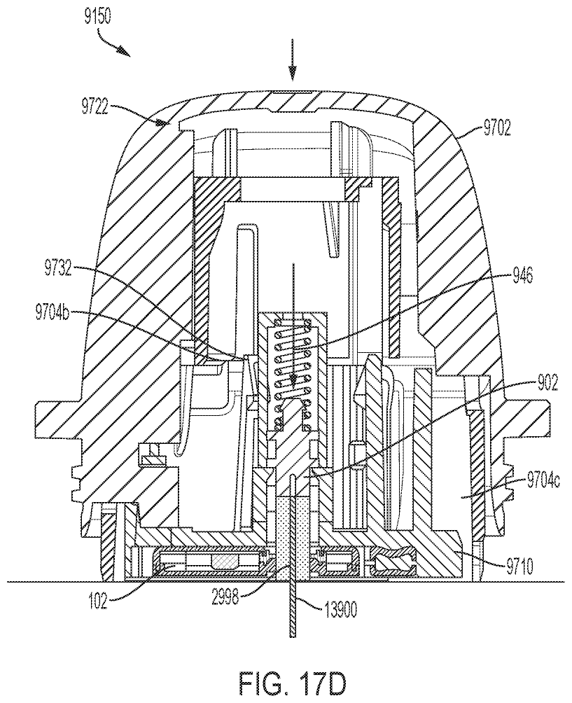

As shown in FIG. 11, sharp retention arms 1618 can be located in an interior of sharp carrier 1102 about a central axis and can include a sharp retention clip 1620 at a distal end of each arm 1618. Sharp retention clip 1620 can have a proximal surface which can be nearly perpendicular to the central axis and can abut a distally facing surface of sharp hub 2516 (FIG. 17A).

### Exemplary Sensor Modules

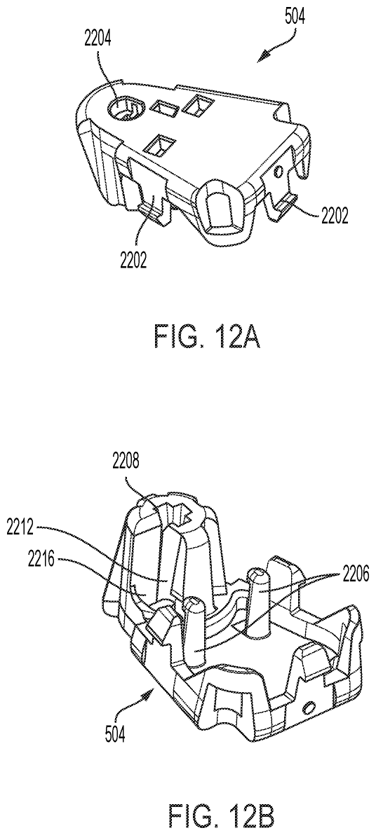

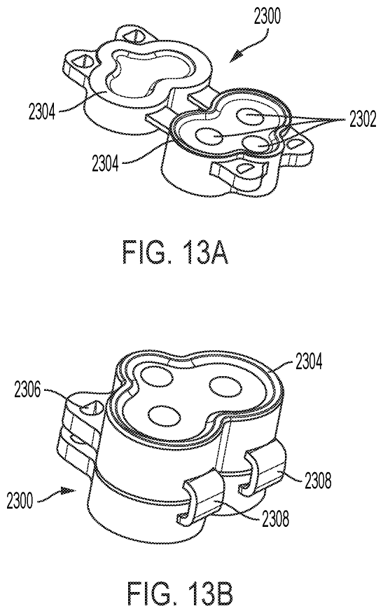

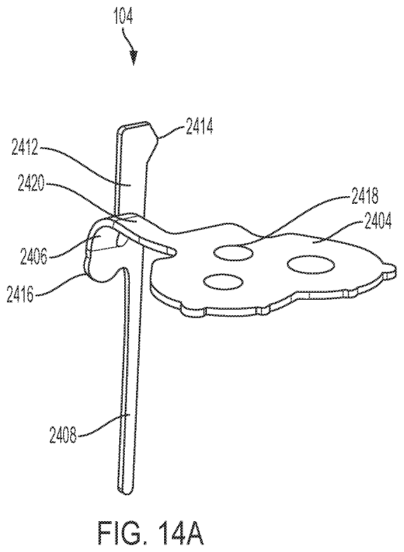

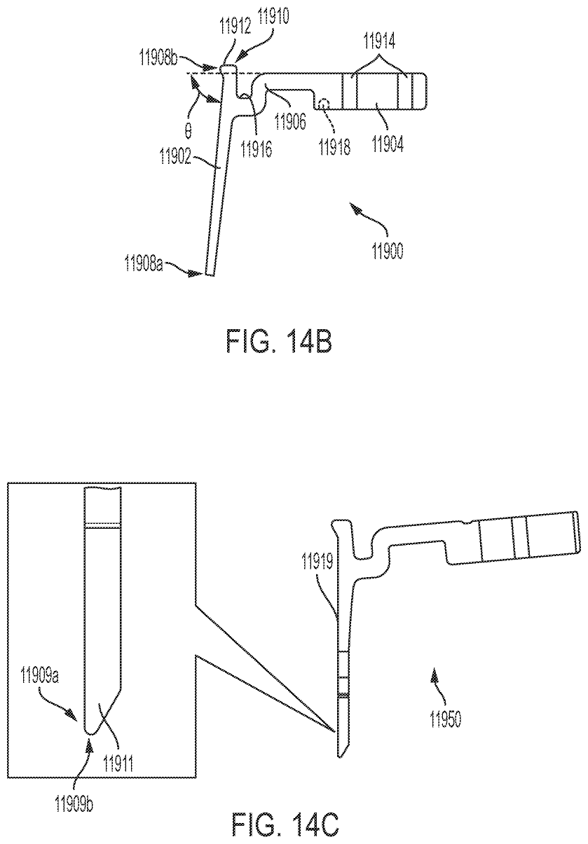

FIGS. 12A and 12B are a top perspective view and a bottom perspective view, respectively, depicting an example embodiment of sensor module 504. Module 504 can hold a connector 2300 (FIGS. 13A and 13B) and a sensor 104 (FIG. 14). Module 504 is capable of being securely coupled with electronics housing 706. One or more deflectable arms or module snaps 2202 can snap into the corresponding features 2010 of housing 706. A sharp slot 2208 can provide a location for sharp tip 2502 to pass through and sharp shaft 2504 to temporarily reside. A sensor ledge 2212 can define a sensor position in a horizontal plane, prevent a sensor from lifting connector 2300 off of posts and maintain sensor 104 parallel to a plane of connector seals. It can also define sensor bend geometry and minimum bend radius. It can limit sensor travel in a vertical direction and prevent a tower from protruding above an electronics housing surface and define a sensor tail length below a patch surface. A sensor wall 2216 can constrain a sensor and define a sensor bend geometry and minimum bend radius.

FIGS. 13A and 13B are perspective views depicting an example embodiment of connector 2300 in an open state and a closed state, respectively. Connector 2300 can be made of silicone rubber that encapsulates compliant carbon impregnated polymer modules that serve as electrical conductive contacts 2302 between sensor 104 and electrical circuitry contacts for the electronics within housing 706. The connector can also serve as a moisture barrier for sensor 104 when assembled in a compressed state after transfer from a container to an applicator and after application to a user's skin. A plurality of seal surfaces 2304 can provide a watertight seal for electrical contacts and sensor contacts. One or more hinges 2208 can connect two distal and proximal portions of connector 2300.

FIG. 14A is a perspective view depicting an example embodiment of sensor 104. A neck 2406 can be a zone which allows folding of the sensor, for example ninety degrees. A membrane on tail 2408 can cover an active analyte sensing element of the sensor 104. Tail 2408 can be the portion of sensor 104 that resides under a user's skin after insertion. A flag 2404 can contain contacts and a sealing surface. A biasing tower 2412 can be a tab that biases the tail 2408 into sharp slot 2208. A bias fulcrum 2414 can be an offshoot of biasing tower 2412 that contacts an inner surface of a needle to bias a tail into a slot. A bias adjuster 2416 can reduce a localized bending of a tail connection and prevent sensor trace damage. Contacts 2418 can electrically couple the active portion of the sensor to connector 2300. A service loop 2420 can translate an electrical path from a vertical direction ninety degrees and engage with sensor ledge 2212 (FIG. 12B).

FIG. 14B is a side view of an example sensor 11900, according to one or more embodiments of the disclosure. The sensor 11900 may be similar in some respects to any of the sensors described herein and, therefore, may be used in an analyte monitoring system to detect specific analyte concentrations. As illustrated, the sensor 11900 includes a tail 11902, a flag 11904, and a neck 11906 that interconnects the tail 11902 and the flag 11904. The tail 11902 includes an enzyme or other chemistry or biologic and, in some embodiments, a membrane may cover the chemistry. In use, the tail 11902 is transcutaneously received beneath a user's skin, and the chemistry included thereon helps facilitate analyte monitoring in the presence of bodily fluids.

The tail 11902 may be received within a hollow or recessed portion of a sharp (not shown) to at least partially circumscribe the tail 11902 of the sensor 11900. As illustrated, the tail 11902 may extend at an angle Q offset from horizontal. In some embodiments, the angle Q may be about 85°. Accordingly, in contrast to other sensor tails, the tail 11902 may not extend perpendicularly from the flag 11904, but instead at an angle offset from perpendicular. This may prove advantageous in helping maintain the tail 11902 within the recessed portion of the sharp.

The tail 11902 includes a first or bottom end 11908a and a second or top end 11908b opposite the bottom end 11908a. A tower 11910 may be provided at or near the top end 11908b and may extend vertically upward from the location where the neck 11906 interconnects the tail 11902 to the flag 11904. During operation, if the sharp moves laterally, the tower 11910 will help pivot the tail 11902 toward the sharp and otherwise stay within the recessed portion of the sharp. Moreover, in some embodiments, the tower 11910 may provide or otherwise define a protrusion 11912 that extends laterally therefrom. When the sensor 11900 is mated with the sharp and the tail 11902 extends within the recessed portion of the sharp, the protrusion 11912 may engage the inner surface of the recessed portion. In operation, the protrusion 11912 may help keep the tail 11902 within the recessed portion.

The flag 11904 may comprise a generally planar surface having one or more sensor contacts 11914 arranged thereon. The sensor contact(s) 11914 may be configured to align with a corresponding number of compliant carbon impregnated polymer modules encapsulated within a connector.

In some embodiments, as illustrated, the neck 11906 may provide or otherwise define a dip or bend 11916 extending between the flag 11904 and the tail 11902. The bend 11916 may prove advantageous in adding flexibility to the sensor 11900 and helping prevent bending of the neck 11906.

In some embodiments, a notch 11918 (shown in dashed lines) may optionally be defined in the flag near the neck 11906. The notch 11918 may add flexibility and tolerance to the sensor 11900 as the sensor 11900 is mounted to the mount. More specifically, the notch 11918 may help take up interference forces that may occur as the sensor 11900 is mounted within the mount.

FIG. 14C illustrates one embodiment of a sensor 11950 including a modified tail 11919. In some embodiments, the first or bottom end 11909a is chiseled or tapered so as to form a bottom end 11909a having one or more beveled edges. In some embodiments, the bottom end 11909a comprises a single beveled edge 11911. More specifically, the sensor tail 11919 comprises a bottom end 11909a which forms a tip portion 11909b having a sharpened, V-shaped vertex. This sensor design can be advantageous in that reduces the overall footprint of the sensor 11950, and minimizes the size of skin penetration and puncture wound as it dilates the tissue during sensor insertion. Consequently, the risk of ESA created by wound trauma can be mitigated by the sensor's 11950 bottom end 11909a having a V-shaped tip portion 11909b with a sharpened vertex.

Generally, the sensor can be understood as including a tail, a flag, and a neck aligned along a planar surface having a vertical axis and a horizontal axis. The spring-like structure can be created by various orientations of turns in the bend of the neck of a sensor. Between the tail and the flag, the neck can include at least two turns in relation to the vertical axis providing a spring-like structure. The at least two turns can provide, in relation to an axis of the planar surface shared by the tail, the flag, and the neck, overlapping layers of the structure of the neck, where the neck itself remains unbroken. These overlapping turns make up the spring-like structure. In some embodiments, the overlapping layers of the neck are vertically-oriented. In some embodiments, the overlapping layers of the neck are horizontally-oriented.

The turns of the neck can be created by folding the neck of the sensor from a larger neck structure, laser cutting the sensor from a sheet of the material comprising the sensor, printing the sensor having the configuration with turns, stamping the sensor from a sheet of material of which the sensor is composed, or other suitable manufacturing processes for providing precision bends in the neck.

### Example Embodiments of Sharp Modules

FIG. 15A is a perspective view depicting an example embodiment of sharp module 2500 prior to assembly within sensor module 504 (FIGS. 12A and 12B). Sharp 2502 can include a distal tip 2506 which can penetrate the skin while carrying sensor tail in a hollow or recess of sharp shaft 2504 to put the active surface of the sensor tail into contact with bodily fluid. A hub push cylinder 2508 can provide a surface for a sharp carrier to push during insertion. A hub small cylinder 2512 can provide a space for the extension of sharp hub contact faces 1622 (FIG. 11). A hub snap pawl locating cylinder 2514 can provide a distal-facing surface of hub snap pawl 2516 for sharp hub contact faces 1622 to abut. A hub snap pawl 2516 can include a conical surface that opens clip 1620 during installation of sharp module 2500. Further details regarding embodiments of sharp modules, sharps, their components, and variants thereof, are described in U.S. Patent Publication No. 2014/0171771, which is incorporated by reference herein in its entirety and for all purposes.

FIG. 15B depicts another example embodiment of a sharp module 2530, and a callout view of the distal portion thereof, depicting the hollow or recess of sharp shaft 2534 and its distal tip 2536. FIG. 15C further illustrates a close-up side perspective view of distal tip 2536 which can penetrate the skin while carrying sensor tail (not shown) in a hollow or recess of sharp shaft 2534 to put the active surface of the sensor tail into contact with bodily fluid. The U-shaped implement of sharp 2532 is further illustrated in the cross-sectional view shown in FIG. 15D.

Example embodiments of a sharp designed to reduce trauma during a sensor insertion and retraction process will now be described. FIG. 15E is a close-up perspective view of a sharp 2592. In FIG. 15E, the sharp distal tip 2596a includes double beveled edges or transitions 2599 that adjoin at a proximal portion of the distal tip 2596a to form a distal tip 2596a having a vertex 2596b. Specifically, the double beveled edges 2599 are concavely sloped so as to form the sharp distal tip 2596a. Referring to FIG. 15E once more, the distal portion is provided with a concavely angled distal tip 2596a. As shown in FIG. 15F, the angled distal tip 2596a may be provided with a first concavely angled tip portion 2593 and a second steep-angled tip portion 2595. More specifically, the first concavely angled tip portion 2593 slopes into the second steep-angled tip portion 2595. The exemplary configuration, which includes multiple edges and faces, provides a sharp point to reduce penetration force, trauma, and bleeding for the subject. The sharp 2592 has a substantially V-shaped profile in this embodiment.

FIGS. 15G and 15I are cross-sectional view of two embodiments of the sharps described herein. FIG. 15G is a cross-sectional view of a previous embodiment, illustrating a substantially U-shaped cross-sectional area of sharp 2502. FIG. 151 is a cross-sectional view of an embodiment depicting a V-shaped cross-sectional area of sharp 2592, having a vertex 2596a. In some embodiments, the vertex 2596a includes a bottom portion of the cross-sectional area with no sharp edges. Additionally, the bottom portion of the cross-sectional area, unlike some U-shaped embodiments, such as the sharp embodiment illustrated in FIG. 15G, is not flattened. FIGS. 15H and 15J are cross-sectional views of the embodiments depicted in FIGS. 15G and 15I, respectively, illustrating the sharp embodiments supporting a sensor, for example, sensor 11900 or sensor 11950, respectively.

15K is a perspective view depicting an example embodiment of a sharp module 2590 having one or more beveled edges and V-shaped geometry configured to create a smaller opening in the skin relative to other sharps (e.g., sharp 2502 depicted in FIG. 15A). Sharp module 2590 is shown here prior to assembly with sensor module 504 (FIGS. 12A and 12B), and can include components similar to those of the embodiment described with respect to FIG. 15A, including sharp 2592, sharp shaft 2594, sharp distal tip 2596a, hub push cylinder (not shown), hub small cylinder 2652, hub snap pawl 2656 and hub snap pawl locating cylinder (not shown). Similar to the previously described sharp module 2500, and as shown in the callout view also depicted in FIG. 15K, sharp module 2590 can include a sharp shaft 2594 coupled to a distal end of the hub portion 2692 at a proximal end, sensor channel 2598 configured to receive at least a portion of an analyte sensor, such as analyte sensor 11950, for example, and a distal tip 2596a configured to penetrate a skin surface during the sensor insertion process.

In FIG. 15L, a front perspective view of sharp shaft 2594 is depicted, and includes a sharp module 2590 comprising one or more sidewalls 2699 and a longitudinally-extending sensor channel 2598 that together form the sharp's V-shaped cross-sectional area comprising a vertex 2596b. Further, the sensor channel 2598 is configured to receive at least a portion of any analyte sensor which has been described herein, such as analyte sensor 1900 or 11950, for example. In some embodiments, such as the embodiment which is illustrated in FIG. 15L, sensor channel 2598 is configured to receive at least a portion of analyte sensor 11900. According to one aspect of the embodiments, one or all of the sharp portion 2592, the sharp shaft 2594, and/or the sharp distal tip 2596a of a sharp 2592 can comprise one or more concave beveled edges.

Referring to FIGS. 15K and 15L, according to one aspect of the embodiment, one or more sidewalls 2699 that form sensor channel 2598 are disposed along sharp shaft 2594 and are adjacent to the distal tip 2596a. Specifically, the one or more sidewalls 2699 are disposed along the sharp shaft 2594 such that the terminus of the one or more sidewalls 2699 is distal to the terminus of the sensor channel 2598. In some embodiments, for example, the terminus of the one or more sidewalls 2699 extend from the vertex 2596b of the sharp distal tip 2596a. The terminus of the one or more sidewalls 2699 adjoin at the proximal end of the distal tip 2596a so as to form the one or more beveled edges thereof. The one or more beveled edges are configured such that they concavely slope and define the vertex 2596b of the distal tip 2596a. In some embodiments, the vertex 2596b is centrally located at the sharp distal tip 2596a. According to one aspect of the embodiment, a sharp 2592 can be characterized as having a sharpened V-shaped distal tip 2596a with all other edges comprising double beveled edges.

The V-shaped tip portion 2596a of the sharp 2592 is designed such that it provides less surface area and includes a reduced cross-sectional footprint relative to, for example, distal tip 2506 of sharp module 2500. The cross-sectional area of the distal tip 2596a is the smallest cross-sectional area of the sharp module 2590. During insertion, as the sharp 2592 moves into the skin surface, the sharp point geometry and V-shaped cross-sectional area of the sharp 2592, as illustrated in FIGS. 15E-15L, penetrate the skin by making a smaller wound due to the smaller size of the tip portion. With respect to sensor insertion, puncture wounds can contribute to ESA in sensors. In this regard, the embodiments described herein provide for less trauma and, consequently, a reduced risk of ESA result during the sensor insertion process.

Furthermore, it will be understood by those of skill in the art that the sharp 2592 embodiment described herein can similarly be used with any of the sensors described herein, including in vivo analyte sensors that are configured to measure an analyte level in a bodily fluid of a subject. Moreover, the sharp embodiments described herein can similarly be used with in vivo analyte sensors comprising a V-shaped tip. For example, in some embodiments, as shown in FIG. 15K, sharp 2592 can include a sensor channel 2598 configured to receive at least a portion of an analyte sensor 11950 with a V-shaped tip 11909b. Further, the V-shaped tip portion 11909b of the sensor 11950 is adjacent to the sharp 2592 and permits the sharp 2592 to create an insertion path for the sensor 11950. In this embodiment, the sharp design itself, and the positioning of the needle with respect to the sensor can be implemented such that the assembly causes less trauma during the insertion process. The distal section of the sensor body has a width sized to fit within the sensor channel 2598. For example, the V-shaped tip portion 11909b of the sensor 11950 is designed to have a complementary shape to the V-shaped cross-sectional area of the sharp 2592. In this embodiment, the V-shaped tip portion 11909b of the sensor 11950 is optimized such that its tip portion 11909b is co-axial to the vertex 2596b of the sharp 2592 so as to better follow the distal tip 2596a as it penetrates the subject's skin.

### Example Embodiments of Sharpless Applicators

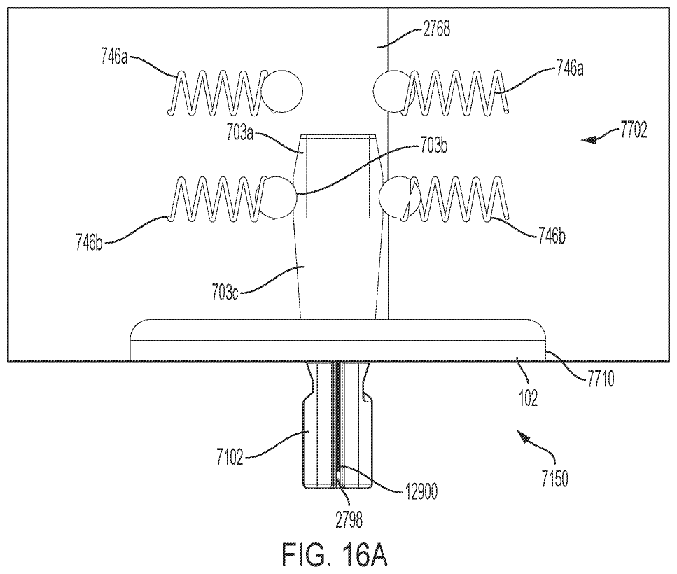

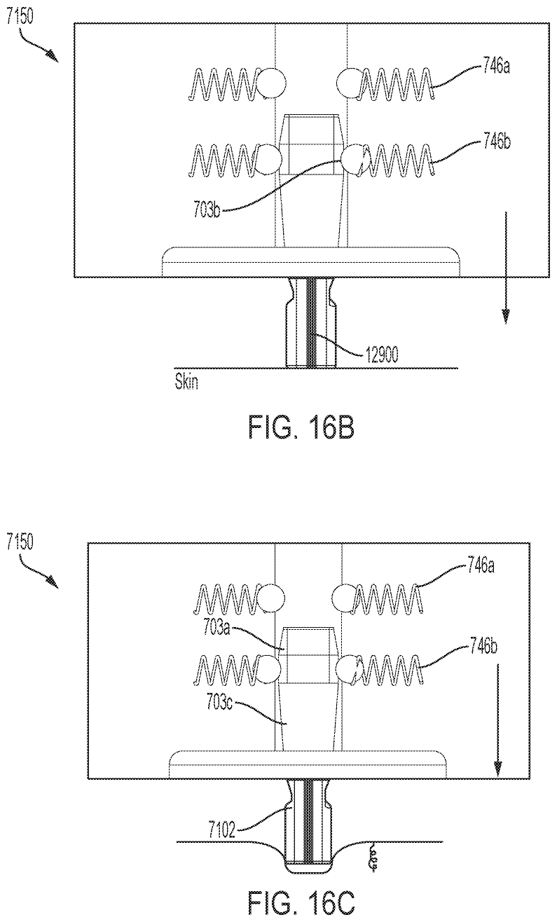

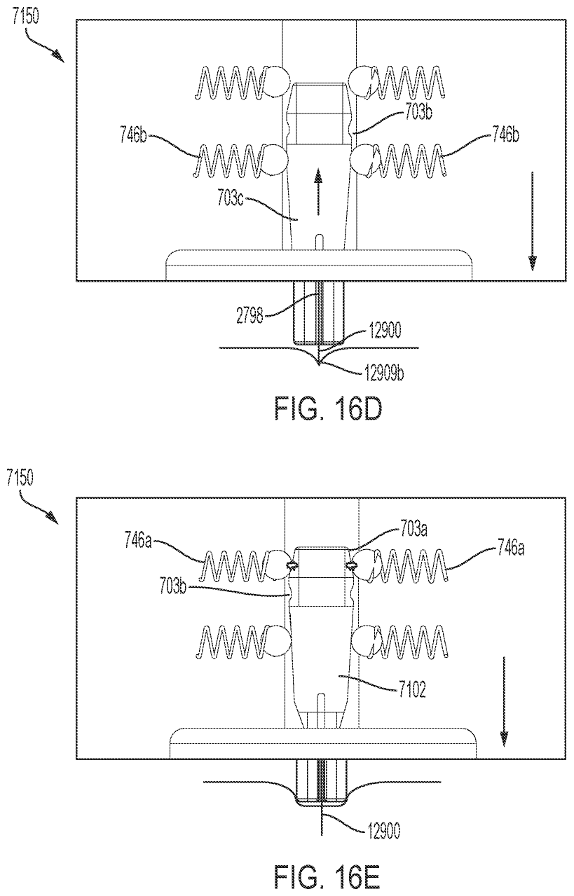

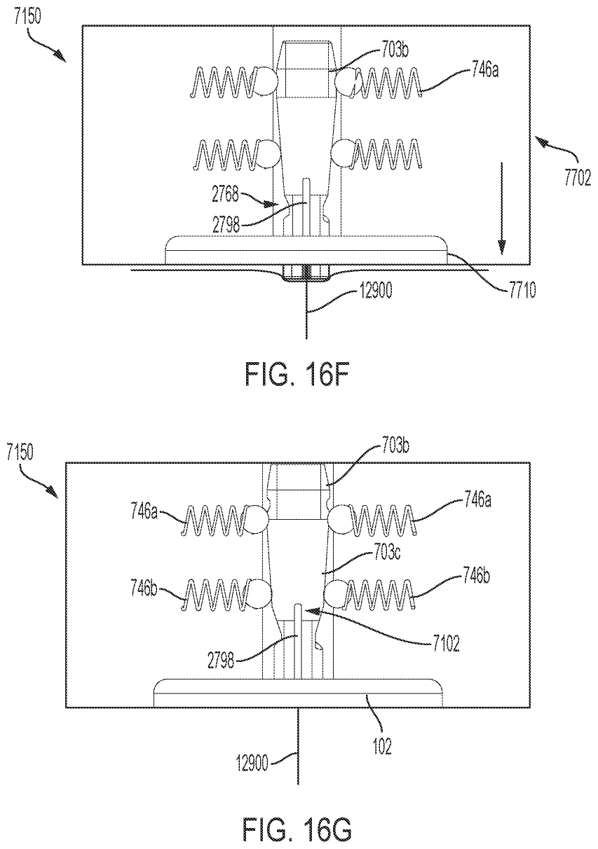

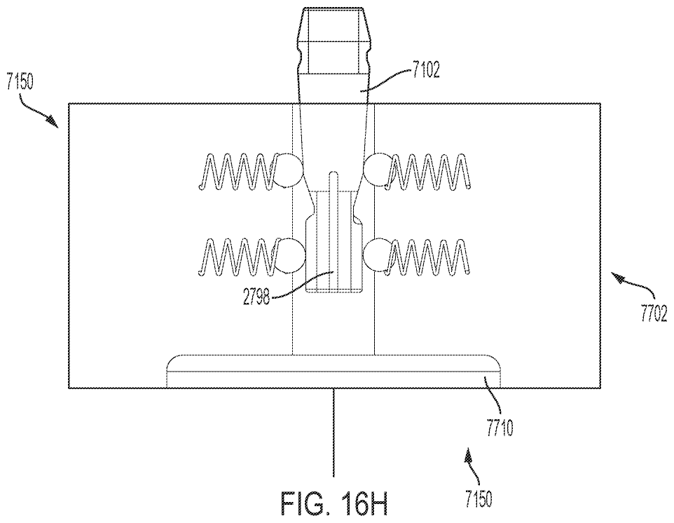

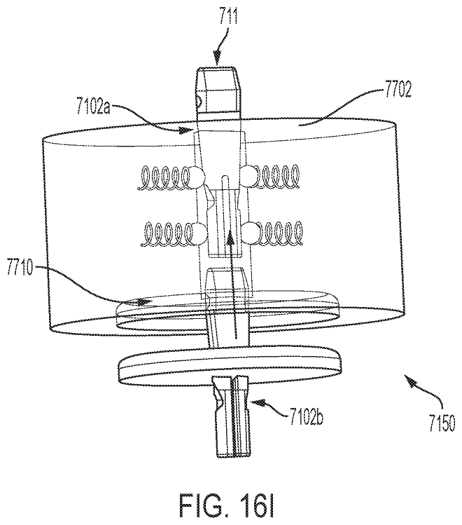

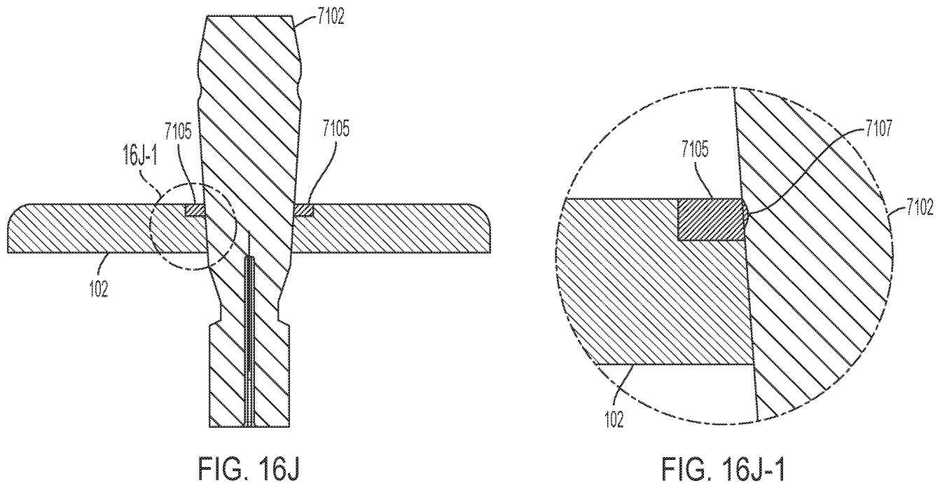

Example embodiments of various sharpless applicators will now be described. Referring first to FIG. 16A, a cross-sectional view of an example embodiment of a sharpless applicator 7150 (in an initial state) is depicted. According to one aspect of some embodiments, sharpless applicator assembly 7150 can include one or more of the following components: housing 7702, which can be movable between a proximal position and a distal position relative to a subject's skin; guide 7102; analyte sensor 12900; device carrier feature 7710 configured to releasably retain sensor control device 102; and a spring loaded system comprising a plurality of spring elements (e.g., spring elements 746a, 746b) which can be configured to engage with a proximal portion of guide 7102. Furthermore, in many embodiments, guide 7102 can be hollow and have a substantially cylindrical shape. In many embodiments, guide 7102 can also include a plurality of ramp surfaces 703a, 703c (e.g., two) with a groove or detent section 703b disposed in between the ramp surfaces.

According to another aspect of some embodiments, device carrier feature 7710 can have a concave or partially concave geometry that is complementary to the shape of sensor control device 102. For example, in some embodiments, device carrier feature 7710 can comprise a hollow round flat cylindrical shape configured to releasably contain sensor control device 102.

Referring still to FIG. 16A, in some embodiments, guide 7102 can comprise a sensor support channel 2798, wherein at least a portion of the sensor support channel 2798 can be disposed in a distal end of guide 7102. In some embodiments, the distal end of guide 7102 can be configured for placement on the skin of the subject. According to some embodiments, a first end of sensor support channel 2798 can terminate at the distal end of guide 7102. In some embodiments, the proximal end of guide 7102 can be hollow. In other embodiments, the proximal end of the guide 7102 can be solid.

According to another aspect of some embodiments, a width of at least a portion of the proximal end of the guide 7102 can be greater than a width of the distal end of guide 7102. Further, in some embodiments, a first ramp 703a can be located at the proximal end of guide 7102, whereas the second ramp 703c can be located distally relative to the first ramp 703a and groove section 703b. According to some embodiments, a width of the groove or detent section 703b of the guide 7102 can be less than the width of the first ramp 703a and the second ramp 703c (at least at the portion which connects the sections).

Referring still to FIG. 16A, according to another aspect of some embodiments, an analyte sensor 12900 can be at least partially disposed within sensor support channel 2798, and supported by guide 7102. Analyte sensor 12900 can include components similar to those of the embodiment described with respect to FIG. 14C. In many embodiments, analyte sensor 12900 comprises a tip portion 12909b (as depicted in FIG. 16D) with sufficient sharpness to initiate and complete insertion without the need for a separate sharp. Accordingly, analyte sensor 12900 can include a predetermined free length so as to provide the necessary sensor stiffness to facilitate effective insertion into the subject's skin. According to one aspect of the embodiments, stiffness of the sensor can be determined by free length of the sensor (e.g., an analyte sensor with a shorter free length will be stiffer). This can mitigate certain effects from “skin tenting,” a phenomenon that occurs when a sharp tip contacts skin and, prior to penetration, the skin deforms inwardly into the body. As a result of “skin tenting,” if the sharp or sensor is not sufficiently stiff, the sharp or sharpened sensor may fail to create a sufficiently large insertion point or misposition the sensor due to deflection.

Referring back to FIG. 16A, according to some embodiments, sharpless applicator 7150 can include an alignment shaft 2768 for maintaining the longitudinal alignment of the guide 7102 such that guide 7102 moves axially within the alignment shaft 2768 while it interfaces with a spring loaded system. In some embodiments, alignment shaft 2768 has a cylindrical geometry. According to one aspect of some embodiments, guide 7102 can be partially disposed within alignment shaft 2768 of the device support 7710 when the sharpless applicator 7150 is in the initial stage. According to another aspect of some embodiments, guide 7102 can be entirely disposed within alignment shaft 2768 after the sharpless applicator 7150 has completed the insertion and retraction steps.

According to another aspect of some embodiments, a plurality of spring elements can be disposed in or along an interior portion of alignment shaft 2768 to facilitate movement or positioning of guide 7102. In particular, the plurality of spring elements can be configured to control a position of guide 7102, increase or decrease the speed of guide 7102 during insertion and/or retraction, and also limit the free length of the sensor during various stages of operation. In this regard, the sensor stiffness can be maintained to aid in effective insertion. In some embodiments, a plurality of low friction rollers, ball-and-plunger sets, or an equivalent thereof, can be utilized to provide spring elements for the spring-loaded system. As shown in FIG. 16A, two pairs of ball-plunger sets 746a, 746b are utilized, an upper ball-plunger set 746a and a bottom ball-plunger set 746b, wherein each set can comprise a first ball-plunger that interacts with a left side of guide 7102, and a second ball-plunger that interacts a right side of guide 7102. Specifically, each ball-plunger set 746a, 746b can comprise a spring with one end anchored along the alignment shaft 2768 (or, alternatively, another stationary structure of sharpless applicator 7150, such as housing 7702), wherein a second end of the spring includes a ball structure configured to engage with a portion or side of guide 7102 as it travels along alignment shaft 2768. According to another aspect of the embodiments, each spring can be in a partially or fully compressed and/or expanded state during various stages of operation of the sharpless applicator 7150. According to yet another aspect of the embodiments, each of the spring elements can be configured to compress and/or expand at different times. For example, in some embodiments, at a certain stage of retraction, upper ball-plunger set 746a can be positioned further inward toward the center of alignment shaft 2768 relative to the bottom ball-plunger set 746b. In this regard, the sharpless applicator assembly retraction mechanism relies at least in part on an upward force of the subject's skin during insertion, combined with the ball-plunger interaction with the ramped surfaces 703a, 703c of the guides 7102 to allow for retraction of the guide 7102 into a container 711 within the housing 7702. Those of skill in the art will also appreciate that the embodiments of the present disclosure can allow for re-usability of sharpless applicator 7150.

FIGS. 16B-16H are various cross-sectional views depicting an example embodiment of a sharpless applicator assembly 7150 during various stages of operation.

FIG. 16B is a cross-sectional view of an example embodiment of a sharpless applicator assembly 7150 for insertion of an analyte sensor 12900 in a subject. In the initial state, the distal end of the guide 7102 is in contact with the subject's skin surface. In this initial state, as shown in FIG. 16B, the bottom ball-plunger set 746b is engaged with the groove or detent section 703b of guide 7102. According to one aspect of the embodiments, the ball portion of each ball-plunger set of the bottom spring elements 746b is in contact with the detent section 703b, and the corresponding spring of each ball-plunger set is in a partially compressed state. As shown in FIG. 16B, a force is applied to sharpless applicator 7150 causing it to move in a distal direction towards the subject's skin. In some embodiments, housing 7702 can move at a speed of between 1 to 1.5 m/s in the distal direction.

FIG. 16C is a cross-sectional view showing sharpless applicator assembly 7150 as applicator 7150 continues moving distally (e.g., in a downward direction) and creates a force in the distal direction against the skin. Meanwhile, according to one aspect of the embodiments, the bottom ball-plunger set 746b exerts a force on groove section 703b of guide 7102, and prevents guide 7102 from moving until a sufficient force in the proximal direction is received. As applicator 7150 continues to move distally, distal end of guide 7102 continues to push down on the subject's skin. As a result, as can be seen in FIG. 16C, the skin surface deforms from the force of guide 7102, and subsequently exerts a counterforce in the proximal direction against guide 7102. Consequently, the bottom ball-plunger set 746b begins to disengage from the groove section 703b.

Turning to FIG. 16D, as applicator assembly 7150 continues to move in a distal direction, the downward force from applicator 7150 further deforms the skin, which in turn increases the skin's counterforce exerted in the proximal direction against guide 7102. The upward force exerted by the skin exceeds the force imparted from the spring loaded system to hold guide 7102 in place. Consequently, bottom ball-plunger set 746b disengages from groove section 703b of guide 7102, as guide 7102 begins to travel in a proximal direction inside alignment shaft. According to another aspect of some embodiments, ramped surface of guide 7102 results in acceleration of guide 7102 in the proximal (e.g., upward) direction, and subsequently exposes the sharp tip portion 12909b of analyte sensor 12900 disposed within the sensor support channel 2798. The acceleration of the guide 7102 increases the velocity of the skin relative to the sensor 12900, as the skin follows guide 7102. In this regard, the sharpless applicator assembly 7150 utilizes the skin deformation, or skin tenting, during insertion as a source of potential energy to further load the guide 7102 and increase relative velocity between skin and sensor. This increase in velocity can aid in insertion effectiveness. As the sharp sensor 12900 protrudes from the sensor support channel 2798, it begins to insert itself into the skin.

FIG. 16E shows the applicator assembly 7150 as it continues moving in a distal direction towards the skin. At the stage illustrated in FIG. 16E, guide 7102 continues to accelerate in the proximal direction until the first ramp 703a engages the upper ball-plunger set 746a, which impedes the motion of guide 7102. This ensures that sensor length during insertion is small enough to maintain the desired stiffness for effective insertion and avoid sensor bending. At this stage, the contact between the guide 7102 and the upper ball-plunger set 746a increases the force applied to skin, thereby causing further skin deformation. As this occurs, the counterforce created by the skin overcomes the force from the ball-plunger sets 746a, 746b, causing guide 7102 to accelerate in the proximal direction (e.g., away from the skin).

FIG. 16F shows the applicator assembly 7150 as it continues moving downward after sensor insertion. Guide 7102 continues to travel in a proximal direction into housing 7702 due to the counterforce generated by the skin. According to some embodiments, at this stage, sensor insertion can continue with more of the sensor length entering the skin of the subject. Further, at this stage, the upper ball-plunger set 746a sits in the guide grooves 703b. In this stage, the guide 7102 is still partially protruding from its distal end and is not fully retracted into the applicator 7150.

FIG. 16G illustrates sharpless applicator assembly 7150 in a sensor fully-inserted stage. In some embodiments, the motion of housing is slowed or stopped by sensor control device 102 contacting the skin. According to one aspect of the embodiments, at the stage depicted in FIG. 16G, analyte sensor 12900 has reached the desired insertion depth in the subject's skin. In some embodiments, distal end of guide 7102 can be flush with bottom surface of sensor control device 102. As shown in FIG. 16G, guide groove section 703b has now been advanced to a position proximal to the upper ball-plunger set 746a. As such, both ball-plunger sets 746a, 746b are in contact with the second ramp 703c of guide 7102. This can impart a force on guide 7102 in a proximal direction, thereby pushing guide 7102 further into the housing 7702 and away from device carrier 7710. With respect to the guide ramp shapes and ball-plunger forces, those of skill in the art will appreciate that certain embodiments of the guide ramps and ball-plungers can be dimensioned and configured to provide optimization for increased axial force in either a distal or proximal direction to ensure insertion and/or retraction. For example, if certain guide ramp surfaces are more sloped, this can cause an increase in insertion and/or retraction speed. Alternatively, certain guide ramp shapes may increase the initial acceleration.

FIG. 16H is a cross-sectional view showing the sharpless applicator assembly 7150 in a retraction state. As depicted in FIG. 16H, sensor 12900 has been inserted into the subject's skin to the desired insertion depth, and guide 7102 has been retracted fully into the applicator 7150 and away from user access. The sharpless applicator assembly 7150 is further advantageous because it does not contain a sharp and is therefore not considered a biohazard.

In addition, as seen in FIG. 16I, according to some embodiments, sharpless applicator assembly 7150 can include a container 711 either coupled with or inside applicator 7150. According to some embodiments, container 711 can be configured to collect used guides. In some embodiments where sharpless applicator assembly 7150 is reusable, the user may reload a new guide and sensor control device into the distal end of the applicator. By doing this, a force in the proximal direction is exerted on the used guide 7102a, causing it to push upwards into the applicator 7150 until it is collected in the container 711. In some embodiments, the container 711 is designed and configured to collect a plurality of used guides. Alternatively, the used guide 7102a may be ejected from an aperture at the proximal surface of the applicator 7150 every time a user loads a new guide 7102b.

FIG. 16J depicts a guide-sensor control device assembly, which can be configured to be loaded into a reusable sharpless applicator, such as those depicted in FIGS. 16A to 16I. As can be seen in call-out FIG. 16J-1, to prevent contaminants from entering an interior of sensor control device 102, a seal 7105 can be disposed at an interface between guide 7102 and sensor control device 102. According to one aspect of the embodiments, seal 7105 can comprise an overmolded elastomeric material. In some embodiments, seal 7105 can comprise a single annular ring configured to interface with guide 7102. In other embodiments, seal 7105 can comprise a plurality of discrete elastomeric components configured to interface a corresponding plurality of locations along guide 7102. According to another aspect of some embodiments, seal 7105 can comprise at least one concave surface 7107 configured to interface with guide 7102. In some embodiments, sensor control device 102 can comprise a housing constructed from a hard plastic material different from the elastomeric material of seal 7105. In other embodiments, housing of sensor control device 102 can be constructed from the same elastomeric material of seal 7105, such that the housing and seal have a unitary construction. Those of skill in the art will appreciate that guide 7102, or at least some portions thereof, can comprise an elastomeric material or have one or more concave surfaces to provide for a barrier against contaminants entering the interior of sensor control device 102.

Another example embodiment of a sharpless applicator 9150 is illustrated in cross-section in FIG. 17A. Referring to FIG. 17A, various components of sharpless applicator 9150 will now be described. In particular, FIG. 17A depicts a cross-sectional view of a sharpless applicator 9150 in an initial state, shown along with two call-out views (FIG. 17A-1 and FIG. 17A-2), wherein sharpless applicator 9150 can comprise the following components: housing 9702, sheath 9704, guide 902, retraction spring 946, and device carrier 9710. FIG. 17A also depicts sensor control device 102 and sensor 13900 disposed entirely within sharpless applicator 9150. Those of skill in the art will understand that sharpless applicator 9150 can include any of the embodiments of housings, sheaths, device carriers, and/or analyte sensors described herein, or in other publications which have been incorporated by reference.

FIG. 17A illustrates sharpless applicator 9150 in an initial state, prior to insertion, in which housing 9702 is in a proximal position with respect to sheath 9704. According to an aspect of the embodiments, sheath 9704 is slidably coupled with and partially positioned within housing 9702. Although sheath 9704 and housing 9702 are shown in FIG. 17A as having a generally cylindrical geometry, those of skill in the art will appreciate that other geometries can be utilized. According to another aspect of the embodiments, as best seen in call-out FIG. 17A-2, spring 946 can comprise a distal end of the spring 946 in contact with a proximal end of guide 902, and a proximal end of spring 946 in contact with a spring retention element 9712. In some embodiments, spring retention element 9732 can be a proximally extending arm that protrudes from device carrier 9710, as described in further detail below. In other embodiments (not shown), spring retention element 9732 can comprise a feature of sheath 9704.

Referring still to FIG. 17A, according to an aspect of some embodiments, in the initial configuration, the retraction spring 946 may be in a semi-compressed state, i.e., not fully compressed, nor fully expanded, and pre-loaded against the guide 902 while housing 9702 is disposed proximally from sheath 9704. In other embodiments, retraction spring 946 may be fully compressed.

According to another aspect of some embodiments, sheath 9704 generally encloses or defines a cavity within which guide 902 and device carrier 9710 are moveable from a proximal position entirely within the applicator to a distal position. In many of the embodiments, device carrier 9710 is configured to releasably retain a sensor control device 102 having a distal surface for placement on the skin of the subject. In some embodiments, a guide locating ring (not shown) which receives one end of the guide 902 can be provided. Furthermore, in some embodiments, device carrier 9710 can comprise a proximally-extending latch 9733 (seen in call-out FIG. 17A-2) configured to retain guide 902 in a retracted position after insertion is complete. At least a portion of guide 902 extends through and is engaged with device carrier 9710 and sensor control device 102, and movable therein during insertion (e.g., before retraction is completed). As described above, in some embodiments, device carrier 9710 can also include one or more deflectable arms 9732 for retaining the retraction spring 946 in a compressed or partially compressed state. In some embodiments, the one or more deflectable arms 9732 may also engage with an exterior surface or wall of guide 902. The one or more movable arms 9732 may be maintained in engagement with the guide 9732 when the device carrier 9710 is in the proximal position.

According to another aspect of the embodiments, guide 902 can have a hollow and/or substantially cylindrical shape, and can include a sensor support channel or slot 2998, at least a portion of which is located in a distal portion of guide 902. In some embodiments, sensor support channel or slot 2998 can include a distal end that does not extend beyond the distal portion of guide 902. The proximal portion of guide 902 can be hollow and include a conical or partially conical surface.

According to another aspect of some embodiments, analyte sensor 13900 is at least partially disposed within sensor support channel or slot 2998 and, as best seen in call-out FIG. 17A-1, supported by one or more supporting walls of guide 902. The sensor tail of the analyte sensor 13900 may be disposed in the sensor support channel 2998. Analyte sensor 13900 can comprises a tip portion 13909b with sufficient sharpness so as to initiate and complete insertion without the need for a separate sharp. In some embodiments, sensor 13900 comprises a sensor tail 13909b having a bottom end with a V-shaped tip portion. Additionally, analyte sensor 13900 further comprises sufficient free length so as to provide the necessary sensor stiffness to facilitate effective insertion into the subject's skin. Stiffness of the sensor is determined by free length of the sensor. For example, an analyte sensor with a shorter free length will be stiffer. Analyte sensor 13900 can include components similar to those of the embodiment described with respect to FIG. 14C.

FIG. 17B illustrates applicator 9150 in cross-section as a user applies a force in a distal direction to housing 9702 (as indicated by the downward arrow). In some embodiments, a predetermined minimum force must be used so that attachment snap 9726 advances past detent 9724. After detent 9724 has been overcome, e.g., snap 9726 is radially displaced, and further depression of housing 9702 with respect to sheath 9704 further causes guide 902, device carrier 9710, and sensor control device 102 to continue to advance from a proximal position towards a distal position towards the skin. In this stage, the distal end of the guide 902 is protruding further distally than the distal surface of sensor control device 102.

Referring next to FIG. 17C, applicator 9150 is depicted as the distal portion of guide 902 and sensor 13900 make contact with the subject's skin surface. Subsequently, the sensor's sharpened tip portion 13909b pierces the skin, while sensor 13900 is supported by guide 902, and inserts a sensor insertion portion of sensor 13900 into the subject's skin, S. In some embodiments, during this phase, the interior surface of proximal portion 9704a of the sheath 9704 remains engaged with the carrier arms 9732 to prevent radial displacement of the arms 9732, and thus maintains retraction spring 946 in a compressed or partially compressed state.

Referring next to FIG. 17D, applicator 9150 is depicted as sensor 13900 reaches its insertion depth, and device carrier 9710 and sensor control device 102 have reached the distal position. In some embodiments, sensor control device 102 includes an adhesive pad that engages the skin surface, S, of the subject at this stage.

According to another aspect of the embodiments, spring retention element 9732 causes retraction spring 946 to expand and retract guide 902 to its retracted position. In some embodiments, for example, spring 946 is a passive element that, during retraction, expands and can return guide 902 to its initial position, or alternatively, be captured by a latch 9733 (FIG. 17A) at some point in between which would allow the guide 902 to be at a different position than its initial position. According to many of the embodiments, sensor 13900 is maintained in its inserted position and sensor control device 102 is left attached to the skin.

Referring still to FIG. 17D, in some embodiments, retraction can be activated when carrier arms 9732 are advanced distally beyond shelf 9704b of sheath and clear a support wall. This allows carrier arms 9732 to deflect radially outwardly into the larger diameter distal portion 9704c of the sheath 9704. When carrier arms 9732 deflect outwardly, shoulder portions of carrier arms 9732 are no longer in an interference relationship with the guide 902, which can be retracted in a proximal direction to its retracted position within applicator 9150. According to another aspect of some embodiments, the retraction step can also be aided by the counterforce created by the skin (and skin deformation), as described earlier with respect to FIG. 16A to 16J.

According to another aspect of some embodiments, housing 9702 is maintained in the distal position by a lock-out feature. In some embodiments, for example, a sheath snap of the sheath 9704 can move up to lock over feature 9722 of the housing 9702. Now the housing 9702 and the sheath 9704 can no longer slidably move with respect to each other, and provides an indication to a user that the inserter has been used.

With respect to retraction spring 946, it should be noted that although compression springs are shown in FIGS. 17A-17D, those of skill in the art will appreciate that other types of springs can be utilized in any of the embodiments described herein, including but not limited to torsion springs, disc springs, leaf springs and others. Furthermore, those of skill in the art will understand that insertion efficiency of the applicator embodiments described herein can be changed by changing insertion speed, sensor length, material or shape, and guide reaction force and fit with sensor during insertion. Additionally, insertion efficiency may be changed by pre-penetrating the skin, as well. Similarly, those of skill in the art will understand that insertion and retraction speeds of the applicator embodiments described herein can be changed by changing the stiffness or length of the retraction spring, and the insertion force and travel length of the sensor.

In some embodiments, the applicator may be a powered applicator and comprise an insertion spring to drive the sensor (and other elements) into the skin of the subject. In the powered applicator embodiment, the insertion spring can be compressed and pre-loaded before it is fired.

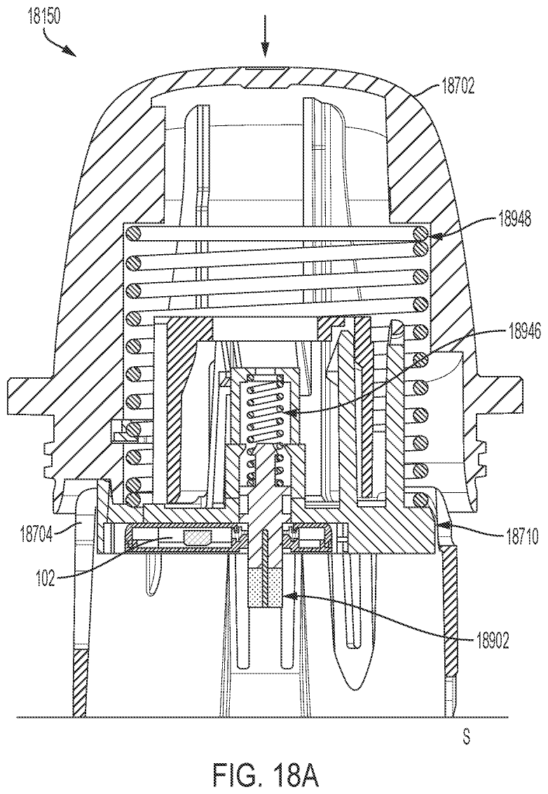

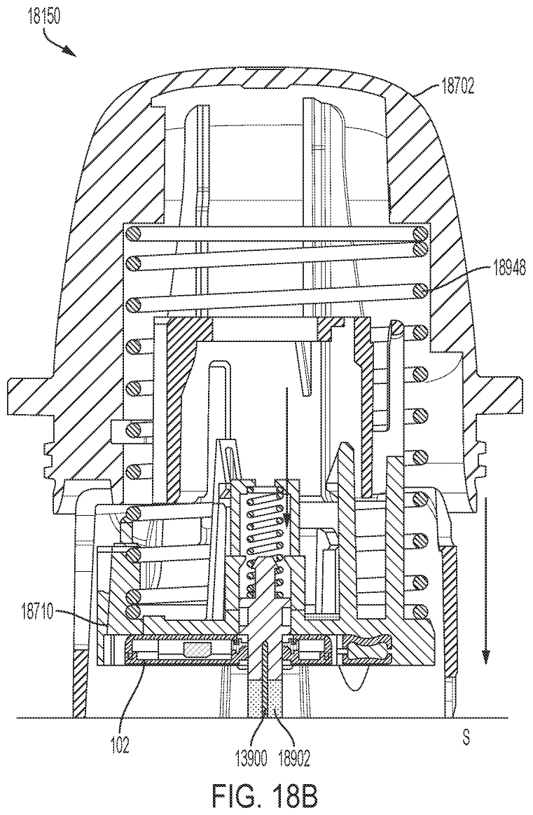

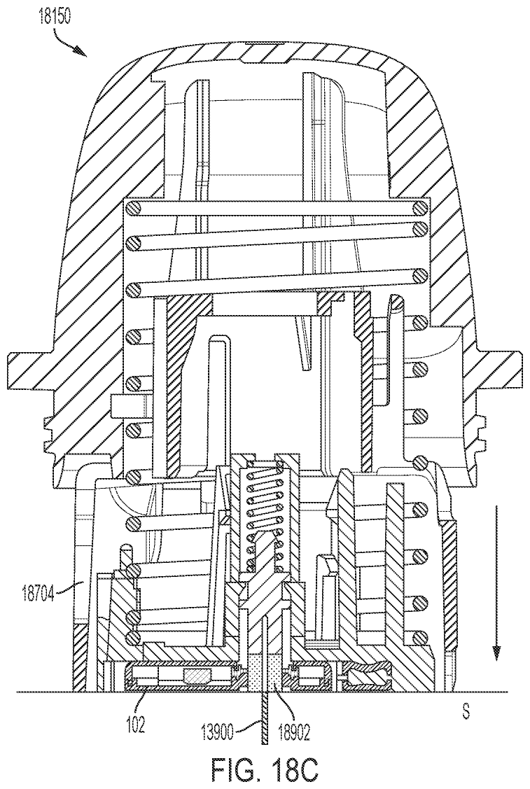

FIGS. 18A to 18C depict cross-sectional views of a powered and sharpless applicator 18150 in various stages of operation. In many respects, applicator 18150 operates in a similar manner to applicator 9150, as described with respect to FIGS. 17A to 17D. For example, applicator 18150 can include many of the same components to applicator 9150, including a housing 18702, sheath 18704, retraction spring 18946, device carrier 18710, and guide 18902, each of which operates in a substantially similar manner to its corresponding component in applicator 9150 (FIGS. 17A to 17D). According to one aspect of the embodiments, however, applicator 18150 further comprises an insertion spring 18948 configured to facilitate a powered (or partially powered) insertion of the sensor.

Referring first to FIG. 18A, applicator 18150 is depicted in an initial stage, where user begins to apply a force upon housing 18702 in a distal direction (as indicated by the downward arrow above applicator 18150). According to one aspect of the embodiments, before the force is applied, carrier 18710, guide 18902, and sensor control device 102 are in a proximal position entirely within applicator 18150. According to another aspect of the embodiments, both insertion spring 18948 and retraction spring 18946 are in a compressed or partially compressed state. Referring next to FIG. 18B, insertion spring 18948 is shown in an activated state, which causes device carrier 18710, sensor control device 102, and guide 18902 to advance from the proximal position to a distal position (as indicated by the downward arrow to the right of applicator 18150). At this stage, guide 18902 is in contact with the subject's skin, S, and configured to support sensor 13900 during insertion. In some embodiments, insertion spring 18948 is triggered when housing 18702 is advanced a predetermined distance in the distal direction in response to the application of force in the distal direction. For example, housing 18702 can be configured to cause a retention element (not shown) to disengage from insertion spring 18948, causing insertion spring 18948 to expand. In some embodiments, insertion spring 18948 can be activated by a separate button, for example, that can only be depressed if the housing is advanced a predetermined distance in the distal direction. Those of skill in the art will appreciate that other mechanisms for activating insertion spring 18948 can be utilized and are within the scope of the present disclosure.

Referring next to FIG. 18C, sensor 13900 is depicted after it reached the insertion depth. At this stage, sensor control device 102 has also reached the distal position. In some embodiments, sensor control device 102 can include an adhesive patch or an adhesive distal-facing surface that is configured to adhere the sensor control device 102 to the subject's skin, S. According to another aspect of the embodiments, retraction spring 18946 has been activated and retracts guide 18902 in the proximal direction to a retracted position. In some embodiments, retracted position of guide 18902 is the same as the initial proximal position of guide 18902, as shown in FIG. 18A. In other embodiments, the retracted position of guide 18902 can be different from the initial proximal position of guide 18902, as shown in FIG. 18A. Subsequently, the applicator 18150 can be removed from the user's skin, S, leaving behind the sensor control device 102 and sensor 13900.

In some embodiments, either or both of sheath 18704 and/or guide 18902 can be locked into the retracted position. As described with respect to FIGS. 17A to 17D, applicator 18150 can include lock-out mechanisms to provide an indication to the user that the applicator has been used.

### Example Embodiments of an Insertion Apparatus

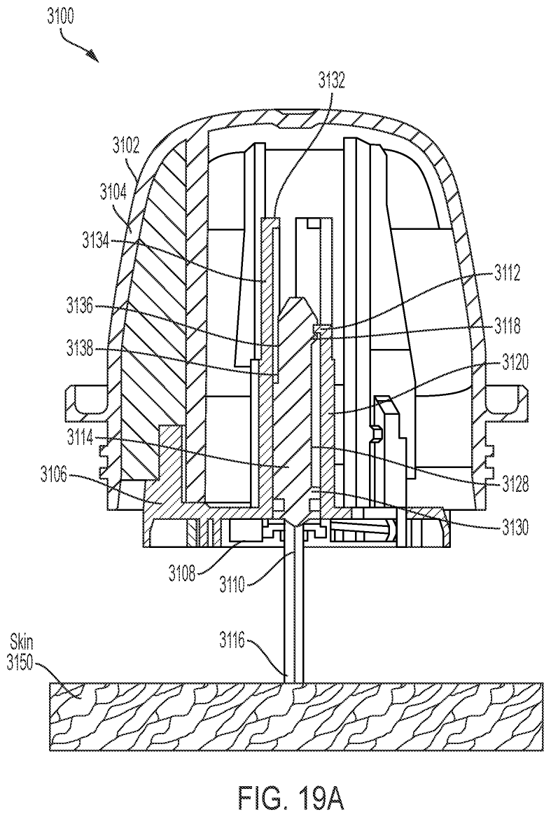

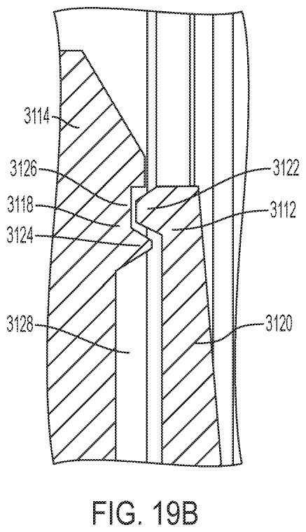

Referring to FIGS. 19A to 21B, according to a first embodiment of the present disclosure, an insertion apparatus 3100 is provided.

Referring in particular to FIG. 19A, the insertion apparatus 3100 is shown. The insertion apparatus 3100 is generally provided for inserting a sensor 3110, such as an analyte sensor, into skin 3150 of a subject. The insertion apparatus 3100 may otherwise be referred to as an applicator. Those of skill in the art will understand that the insertion apparatus 3100 can include any of the embodiments of applicators, housings, sheaths, device carriers, and/or sensors described herein. In some embodiments, the insertion apparatus 3100 may be similar to the sensor applicator 150 as described herein, and the insertion apparatus 3100 may comprise one or more features described in relation to the sensor applicator 150. In some embodiments, the insertion apparatus 3100 may be similar to the sharpless applicator 7150 as described herein, and the insertion apparatus 3100 may comprise one or more features described in relation to the sharpless applicator 7150.

The insertion apparatus 3100 is arranged so that the sensor 3110 may be inserted in the distal direction to the skin. The distal direction is preferably defined as the direction of insertion of the sensor 3110 towards the skin 3150. In the first embodiment, the distal direction is along the length of the insertion apparatus 3100. The proximal direction is defined as the opposite direction to the distal direction and is the direction away from the skin. The axial direction (or axial axis) comprises the distal direction and the opposite proximal direction. The lateral direction is the direction perpendicular to the distal and proximal directions, and in the first embodiment is the direction along the width of the insertion apparatus 3100.

The insertion apparatus 3100 comprises a housing 3102. The housing 3102 is generally provided for containing components of the insertion apparatus 3100 within a structure. The housing 3102 may otherwise be referred to as a body.

The housing 3102 comprises a cover 3104. The cover 3104 is generally provided for allowing a user to grip the housing 3102 by the cover 3104 to aid insertion of the sensor 3110 into the skin 3150. The cover 3104 may otherwise be referred to as a handle. In some embodiments, the cover 3104 may be similar to the housing 702 as described herein, and the cover 3104 may comprise one or more features described in relation to the housing 702.

The housing 3102 also comprises a device carrier 3106. The device carrier 3106 is generally provided for holding a sensor control device 3108 including the sensor 3110. The device carrier 3106 may otherwise be referred to as a puck carrier. In some embodiments, the device carrier 3106 may be similar to the device carrier 710 as described herein, and the device carrier 3106 may comprise one or more features described in relation to the device carrier 710.

The device carrier 3106 is coupled to the cover 3104. In other words, the device carrier 3106 is connected to the cover 3104. The device carrier 3106 is coupled to the cover 3104 such that movement of the cover 3104 causes movement of the device carrier 3106 and vice versa. In the first embodiment, the device carrier 3106 is rigidly coupled to the cover 3104.

The insertion apparatus 3100 also comprises a sensor control device 3108. The sensor control device 3108 may generally be provided for controlling the sensor 3110 through electronics. The sensor control device 3108 may otherwise be referred to as an on-body unit (OBU) or a puck. In some embodiments, the sensor control device 3108 is configured to receive signals from the sensor 3110, such as data indicative of the sensor measurements, such as analyte concentration. In some embodiments, the sensor control device 3108 may process and/or transmit sensor data, for example to a receiver device, such as the receiver 120 as described herein. In some embodiments, the sensor control device 3108 may be similar to the sensor control device 102 as described herein, and the sensor control device 3108 may comprise one or more features described in relation to the sensor control device 102.

The insertion apparatus 3100 also comprises a sensor 3110. In particular, the sensor control device 3108 comprises the sensor 3110. In the first embodiment, the sensor 3110 is an analyte sensor for inserting into the skin 3150 of a user. In particular, in the first embodiment, the sensor 3110 is a glucose sensor for monitoring glucose from bodily fluids of a user through insertion into the skin 3150. In some embodiments, the sensor 110 may be similar to the sensor 104 as described herein, and the sensor 110 may comprise one or more features described in relation to the sensor 104. In other embodiments, the sensor 110 may be similar to the sensors 11900, 11950, 12900 described herein, and the sensor 110 may comprise one or more features described in relation to the sensors 11900, 11950, 12900. In particular, the sensor 110 may be implemented with features of analyte sensor 12900 such as a tip portion to aid insertion.

The sensor control device 3108 comprises sensor electronics. The sensor electronics can be used to operate the sensor 3110, and process and/or transmit signals generated by the sensor 3110. In some embodiments, the sensor electronics may be similar to the sensor electronics 160 as described herein, and the sensor electronics of the sensor control device 3108 may comprise one or more features described in relation to the sensor electronics 160.

The sensor control device 3108 comprises an adhesive patch. The adhesive patch allows the sensor control device 3108 to be attached to the skin 3150 of the subject when the sensor is inserted into the skin 3150 to hold the sensor and the sensor control device 3108 in position together. In some embodiments, the adhesive patch may be similar to the adhesive patch 105 described herein, and the adhesive patch of the sensor control device 3108 may comprise one or more features described in relation to the adhesive patch 105.

The housing 3102 is configured to releasably retain the sensor control device 3108. In particular, the device carrier 3106 is configured to releasably retain the sensor control device 3108. The device carrier 3106 is configured to retain the sensor control device 3108 in position within the housing 3102 when the sensor control device 3108 is loaded into the device carrier 3106. The device carrier 3106 is then configured to release the sensor control device 3108 onto the skin 3150 of the user. In some embodiments, the insertion apparatus 3100 may be provided without a sensor control device 3108, and instead may be configured to receive a sensor control device 3108.

The sensor 3110 is arranged to protrude from the sensor control device 3108. In particular, the sensor 3110 is arranged to protrude from below the sensor control device 3108 in the distal direction. This allows the sensor 3110 to be inserted into the skin 3150 of the subject before the surface of the sensor control device 3108 contacts the skin 3150, which would otherwise block further insertion of the sensor 3110. In particular, the protruding sensor 3110 allows the sensor 3110 to be inserted into the skin 3150 before the adhesive patch contacts the skin 3150. After insertion of the sensor 3110, the sensor control device 3108 can be released by the device carrier 3106 onto the skin 3150.

The sensor 3110 is configured to penetrate the skin 3150 of the subject. In other words, the sensor 3110 is sufficiently sharp to penetrate the skin 3150 under operation of the insertion apparatus 3100. In particular, the sensor 3110 is configured to penetrate the skin 3150 without requiring a needle. This is achieved at least in part by virtue of the stored potential energy, as set out herein. The sensor 3110 may otherwise be referred to as a needle-free sensor 3110. Accordingly, the insertion apparatus 3100 may otherwise be referred to as a needle-free insertion apparatus or a needle-free applicator.

In the first embodiment, the sensor 3110 has a pointed tip. This makes the sensor 3110 sufficiently sharp for insertion of the sensor 3110 into the skin 3150 without a needle.

The housing 3102 comprises an interface element 3112. In the first embodiment, the device carrier 3106 comprises the interface element 3112. In other embodiments, the interface element 3112 is arranged elsewhere on the housing 3102. For example, in one embodiment, the interface element 3112 is arranged on the cover 3104. In that case, the cover 3104 comprises the interface element 3112.

The insertion apparatus 3100 also comprises a guide 3114. The guide 3114 has a generally elongate shape. In the first embodiment, the guide 3114 has a generally cylindrical shape. The guide 3114 is arranged at least partially within the housing 3102. The guide 3114 comprises a distal portion and a proximal portion. The proximal portion is arranged within the housing 3102. In the first embodiment, the distal portion is arranged externally of the housing 3102. In the first embodiment, the distal portion is narrower than the proximal portion. However, those skilled in the art will appreciate that the guide 3114 may comprise other shapes.

The guide 3114 is configured to support at least a portion of the sensor 3110. In some embodiments, the guide 3114 can comprise a sensor support channel, wherein at least a portion of the sensor support channel can be disposed in the distal portion of the guide 3114. In some embodiments, a first end of sensor support channel can terminate at the distal end of guide 3114. In some embodiments, the proximal portion of guide 3114 can be hollow. In other embodiments, the proximal portion of the guide 3114 can be solid.

The distal portion of the guide 3114 is generally provided for supporting the sensor 3110. In the first embodiment, the distal portion is hollow for receiving the sensor 3110. In this way, the guide 3114 can guide the sensor 3110 towards the skin 3150 for insertion. This allows the guide 3114 to support the sensor 3110 as it travels towards the skin 3150, stabilizing the position of the sensor 3110. This helps insert the sensor 3110 properly. In particular, this helps insert the sensor 3110 at the desired location, in the desired orientation, and smoothly with minimal impact to the user.

The guide 3114 is movable relative to the housing 3102. In other words, the guide 3114 is not rigidly connected to the housing 3102. Therefore, when the housing 3102 is moved, the guide 3114 does not necessarily move. In particular, the housing 3102 is slidable in the proximal/distal direction relative to the guide 3114. The proximal portion is generally provided for engaging with the housing 3102. In particular, the proximal portion is configured to interact with the interface element 3112.

The guide 3114 comprises a distal end 3116. In some embodiments, the distal end 3116 of the guide 3114 can be configured for placement on the skin 3150 of the subject. The distal end 3116 is arranged at the end of the distal portion. The distal end 3116 is configured to engage the skin 3150 to allow distal movement of the housing 3102 relative to the guide 3114. In other words, the distal end 3116 of the guide 3114 abuts the skin 3150. The distal end 3116 abutting the skin 3150 prevents the guide 3114 from moving distally under a distal force applied to the insertion apparatus 3100. In this case, the housing 3102 can generally move distally relative to the guide 3114, while the guide 3114 is prevented from movement due to the distal end 3116 abutting the skin 3150.

Because the housing 3102 is movable relative to the guide 3114, the housing 3102 can be moved distally over the guide 3114 towards the skin 3150. This withdraws the guide 3114 into the housing 3102, and covers more of the distal portion of the guide 3114.

The guide 3114 comprises a retaining element 3118. The retaining element 3118 is generally provided for engaging with the interface element 3112 to control movement of the housing 3102 relative to the guide 3114. In particular, the retaining element 3118 is configured to initially prevent distal movement of the interface element 3112 of the housing 3102 beyond the retaining element 3118, to retain the interface element 3112 in position and prevent distal movement of the housing 3102 relative to the guide 3114, when an applied distal force is below a threshold.

Because the housing 3102 is otherwise movable relative to the guide 3114, the retaining element 3118 allows the movement of the housing 3102 to be controlled by engaging with the interface element 3112 to limit the movement relative to the guide 3114.

In the first embodiment, the housing 3102 comprises a deflectable arm 3120. In particular, the device carrier 3106 comprises the deflectable arm 3120. In other embodiments, the deflectable arm 3120 is provided in other components of the housing 3102, such as the cover 3104. The deflectable arm 3120 is deflectable which preferably means it can be pivoted, deformed, or otherwise bent out of its default position. This allows tension to be stored within the deflectable arm 3120 when it is deflected out of position. In the first embodiment, the deflectable arm 3120 is made from plastic.

The deflectable arm 3120 extends from a main body of the device carrier 3106 and is arranged to extend generally in the proximal direction within the housing 3102. The deflectable arm 3120 is configured to engage the guide 3114. In particular, in the first embodiment, an end of the deflectable arm 3120 forms the interface element 3112.

Referring to FIG. 19B, the interaction between the interface element 3112 and the retaining element 3118 is shown in more detail.

In the first embodiment, the interface element 3112 comprises a protrusion 3122. The protrusion 3122 is at the end of the deflectable arm 3120. In particular, the protrusion 3122 is at the free end of the deflectable arm 3120. The protrusion 3122 extends laterally from the deflectable arm 3120 towards the guide 3114. The protrusion 3122 is configured to interact with the guide 3114.

The protrusion 3122 has a proximal surface which slopes in a distal direction laterally towards the surface of the guide 3114 towards the apex of the protrusion 3122. The protrusion 3122 also has a contact surface adjacent to the proximal surface which is generally flat and faces the surface of the guide 3114, extending in the distal direction. The protrusion 3122 also has a distal surface opposite the proximal surface, which slopes in a distal direction laterally back away from the surface of the guide 3114 from the contact surface of the protrusion 3122. The contact surface is arranged between the proximal surface and the distal surface. In the first embodiment, the protrusion 3122 forms a generally trapezium shape in cross section extending from the deflectable arm 3120.

In the first embodiment, the retaining element 3118 comprises a projection 3124. The projection 3124 of the guide 3114 extends laterally from the surface of the guide 3114 towards the deflectable arm 3120. The projection 3124 extends laterally outwards from the surface of the guide 3114. The projection 3124 has a proximal surface which slopes in a distal direction laterally away from the surface of the guide 3114 towards the apex of the projection 3124. The projection 3124 also has a distal surface opposite the proximal surface, which slopes in a distal direction laterally back towards the surface of the guide 3114 from the apex of the projection 3124. The projection 3124 therefore has a pointed shape. In the first embodiment, the projection 3124 forms a generally triangular shape in cross section extending from the guide 3114.

The guide 3114 comprises a support surface 3128. The support surface 3128 extends in the distal direction from the retaining element 3118. In particular, the support surface 3128 extends from the projection 3124, and extends from the distal surface of the projection 3124. In the first embodiment, the support surface 3128 is generally flat along the length of the guide 3114.

The distal surface of the projection 3124 slopes until it reaches the support surface 3128 of the guide 3114. In the first embodiment, the distal surface of the projection 3124 and the proximal surface of the projection 3124 have corresponding angles (e.g., the angles are equal in magnitude but in opposite directions when measured from the lateral direction). This means that the slopes have the same gradient. In the first embodiment, the distal surface of the projection 3124 is longer than the proximal surface of the projection 3124. This means that the support surface 3128 is deeper than the recess 3126 towards the center of the guide 3114.

When the protrusion 3122 is in contact with the surface of the guide 3114, such as in the recess 3126, the projection 3124 extends outwards preventing the protrusion 3122 from moving distally past the projection 3124. This allows the projection 3124 of the guide 3114 to retain the protrusion 3122 of the deflectable arm 3120 in place. In other words, this allows the retaining element 3118 to retain the interface element 3112 in place.

In the first embodiment, the interface element 3112 of the housing 3102 is biased against the guide 3114. In other words, the interface element 3112 is forced to push against the guide 3114. In this manner, the interface element 3112 contacts the surface of the guide 3114. Because the interface element 3112 is biased against the guide 3114, this aids the function of the retaining element 3118 because the interface element 3112 is biased preventing the interface element 3112 from deflecting around the retaining element 3118. In the first embodiment, the interface element 3112 is biased by virtue of the tension in the deflectable arm 3120. In other embodiments, a biasing member may be provided to bias the deflectable arm 3120 against the guide. For example, a spring may act against the deflectable arm 3120 to push the deflectable arm against the guide 3114.

In the first embodiment, the geometry of the interface element 3112 and the retaining element 3118 act to further improve the retaining force. The projection 3124 extends underneath the protrusion 3122. The protrusion 3122 of the interface element 3112 extends along the proximal side of the projection 3124 of the retaining element 3118. In particular, the distal surface of the protrusion 3122 extends along the proximal surface of the projection 3124. This overlap provides surface contact defining a distance that is required for the protrusion 3122 to deflect in order to overcome the projection 3124. The retention is also aided by the interface element 3112 being biased against the guide 3114 and resisting the protrusion 3122 deflecting this distance over the projection 3124.

In the first embodiment, the guide 3114 comprises a recess 3126. In particular, the retaining element 3118 comprises the recess 3126. The recess 3126 is arranged adjacent to the projection 3124. In particular, the recess 3126 is arranged proximally of the projection 3124.

This means that the recess 3126 is bounded by the projection 3124 at the distal edge of the recess 3126. In other words, the proximal surface of the projection 3124 is adjacent to the distal edge of the recess 3126. When the interface element 3112 is retained by the retaining element 3118, the projection 3122 is arranged within the recess 3126 and supported by the projection 3124. The recess 3126 aids retention of the interface element 3112 because the projection 3122 can be retained in the recess 3126 while the projection 3124 of the retaining element 3118 also prevents distal movement. The recess 3126 can also inhibit proximal movement of the interface element 3112 due to a proximal edge of the recess 3126. This provides more control over the movement of the housing 3102 relative to the guide 3114.

In the first embodiment, the recess 3126 has a complementary shape to the shape of the protrusion 3122 of the interface element 3112. In particular, the recess 3126 comprises a contact surface between the proximal and distal edges. The contact surface is flat and faces the interface element 3112 and is configured to engage the contact surface of the protrusion 3122. The proximal edge of the recess 3126 comprises a sloped surface which is configured to engage the proximal surface of the protrusion 3122. This prevents the interface element 3112 from moving proximally, allowing the housing 3102 to be kept in place without the guide 3114 separating from the deflectable arm 3122. The distal edge of the recess 3126 adjoins the proximal surface of the projection 3124, which engages the distal surface of the protrusion 3122. In this manner, the recess 3126 is configured to receive the protrusion 3122.

In other embodiments, the retaining element 3118 may comprise a recess 3126 without a projection 3124. In this case, the protrusion 3122 can be held within the recess 3126. The recess 3126 can extend laterally inwards from the surface of the guide 3114. In other embodiments, a recess 3126 is not provided and the projection 3124 can provide the desired retention.

In other embodiments, the interface element 3112 is not in the form of a protrusion 3122. For example, in some embodiments, the interface element 3112 may comprise a recess. In this case, the projection 3124 of the guide 3114 may engage with the recess such that the projection 3124 is retained within the recess of the deflectable arm 3120. The interface element 3112 may also comprise a projection proximally of the recess to further prevent distal movement of the interface element 3112 past the retaining element 3118. In this case, the deflectable arm 3120 may then deflect, releasing the projection 3124 from the recess in order to move the housing 3102 relative to the guide 3114.

In summary, in the first embodiment, the interface element 3112 engages with the retaining element 3118. In particular, the protrusion 3122 of the deflectable arm 3120 is arranged within the recess 3126 of the guide 3114, and the projection 3124 of the guide 3114 prevents distal movement of the protrusion 3122. This prevents distal movement of the interface element 3112 past the retaining element 3118. This thereby controls movement of the device carrier 3106 and thus the housing 3102 relative to the guide 3114. This is the first configuration of the insertion apparatus 3100.

Referring back to FIG. 19A, because the distal end 3116 of the guide 3114 abuts the skin 3150, the retaining element 3118 keeps the housing 3102 in position relative to the skin 3150. In the first configuration, the housing 3102 is arranged away from the surface of the skin 3150. In particular, the sensor control device 3108 is spaced from the skin 3150. In other words, the sensor 3110 is positioned away from the skin 3150.

In the first embodiment, the guide 3114 also comprises a second retaining element 3130. The second retaining element 3130 is arranged distally of the retaining element 3118. In other words, the second retaining element 3130 is arranged on the surface of the guide 3114 aligned with the retaining element 3118 in the distal direction. In particular, the second retaining element 3130 is arranged on the opposite side of the support surface 3128 to the retaining element 3118.

In embodiments with the second retaining element 3130, the retaining element 3118 may be referred to as the first retaining element 3118. Similarly, the projection 3124 of the first retaining element 3118 may be referred to as the first projection 3124, and the recess 3126 of the first retaining element 3118 may be referred to as the first recess 3126.

In the first embodiment, the second retaining element 3130 comprises a second projection. For the avoidance of doubt, the term “second projection” is used to differentiate from the projection 3124 of the first retaining element 3118, rather than the second retaining element 3130 itself comprising two projections.

The second projection extends laterally outwards from the surface of the guide 3114 towards the deflectable arm 3120. The second projection comprises a proximal surface, which slopes in a distal direction laterally away from the surface of the guide 3114. In particular, the second projection extends from the support surface 3128.

In other embodiments, the second projection may additionally comprise a distal surface opposite the proximal surface, where the second projection forms an apex. In some embodiments, the second retaining element 3130 may additionally comprise a second recess. For the avoidance of doubt, the term “second recess” is used to differentiate from the recess 3126 of the first retaining element 3118, rather than the second retaining element 3130 itself comprising two recesses. extending laterally out from the surface of the guide 3114. The second recess may be arranged adjacent to and proximally of the second projection, such as adjacent to the proximal surface.

In the first embodiment, the housing 3102 comprises a second interface element 3132. In the first embodiment, the device carrier 3106 comprises the second interface element 3132. In other embodiments, the second interface element 3132 is arranged elsewhere on the housing 3102. For example, in one embodiment, the second interface element 3132 is arranged on the cover 3104. In that case, the cover 3104 comprises the second interface element 3132.

In the first embodiment, the housing 3102 comprises a second deflectable arm 3134. In particular, the device carrier 3106 comprises the second deflectable arm 3134. In embodiments with the second deflectable arm 3134, the deflectable arm 3120 may be referred to as the first deflectable arm 3120. The second deflectable arm 3134 is arranged on the opposite side of the guide 3114 to the first deflectable arm 3120. The second deflectable arm 3134 may be generally similar to the first deflectable arm 3120, except that the second deflectable arm 3134 has a different height than the first deflectable arm 3120. In particular, the second deflectable arm 3134 is longer than the first deflectable arm 3120. The second deflectable arm 3134 extends further into the proximal direction than the first deflectable arm 3134.

The housing 3102, in particular the device carrier 3106, comprises a plurality of deflectable arms including the first deflectable arm 3120 and the second deflectable arm 3134. In some embodiments, the plurality of deflectable arms may comprise other deflectable arms, for example comprising at least three deflectable arms.

In the first embodiment, the guide 3114 comprises a second support surface 3136. In embodiments with the second support surface 3136, the support surface 3128 may be referred to as the first support surface 3128. The second support surface 3136 is on the opposite side of the guide 3114 to the first support surface 3128. The second support surface 3136 is arranged towards the proximal end of the guide 3114.

In the first embodiment, the guide 3114 comprises a third retaining element 3138. The third retaining element 3138 is arranged proximally of the second support surface 3136.

In the first embodiment, when the insertion apparatus 3100 is in the first configuration, the second interface element 3132 does not engage with the guide 3114. As shown in FIG. 19A, the second interface element 3132 is spaced away from the guide 3114. This is by virtue of the second deflectable arm 3134 being longer than the first deflectable arm 3120 and the guide 3114. In other embodiments, the guide 3114 may extend further in the proximal direction so that the second interface element 3132 engages with the side of the guide 3114. However, the arrangement in the first embodiment allows the guide 3114 to be shorter, which allows the length of the cover 3104 to be shorter as it accommodates movement of the guide 3114.

During operation, when a distal force is applied to the insertion apparatus 3100, such as by the user applying a distal force on the cover 3104, the housing 3102 pushes against the guide 3114. In particular, the interface element 3112 pushes against the retaining element 3118. Because the distal end 3116 of the guide 3114 abuts the skin 3150, the guide 3114 is prevented from moving distally, providing a restoring force against the interface element 3112. Because of the tension in the deflectable arm 3120 biasing the interface element 3112 against the guide 3114, the interface element 3112 is prevented from deflecting over the retaining element 3118. As the user exerts a distal force on the housing 3102, tension builds up in the deflectable arm 3120. If the distal force is below a threshold, then the retaining element 3118 will retain the interface element 3112 in position. In this case, the applied force will cause potential energy to be stored in the deflectable arm 3120. The user will experience a resistance to the force they apply.

If the distal force exceeds a threshold, then the interface element 3112 will deflect over the retaining element 3118 due to the tension in the deflectable arm 3120. This will remove the retaining force on the housing 3102, and allow the interface element 3112 to move past the retaining element 3118 and allow the housing 3102 to move relative to the guide 3114 under the applied force. This allows the housing 3102 to move towards the skin 3150.

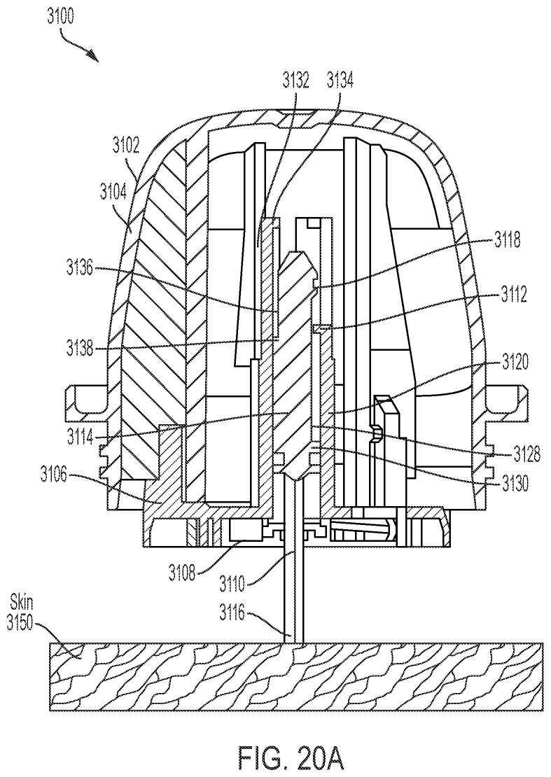

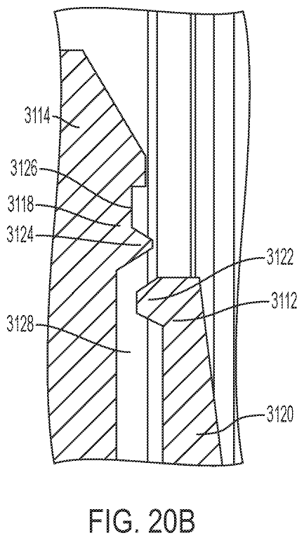

Referring to FIG. 20A, the insertion apparatus 3100 of the first embodiment is shown when the insertion apparatus 3100 is moving from the first configuration to the second configuration. In other words, FIG. 20A shows a point between the first configuration and a second configuration. FIG. 20A is similar to FIG. 19A, except that the housing 3102 has moved distally relative to the guide 3114. In particular, FIG. 20A shows the arrangement after the distal force applied by the user to the housing 3102 exceeds the threshold such that the interface element 3112 deflects over and past the retaining element 3118.

FIG. 20A shows that the interface element 3112 has moved distally past the retaining element 3118. Since the retaining element 3118 is no longer holding the interface element 3112, the housing 3102 has moved distally under the applied force. Therefore, the housing 3102 has moved closer to the skin 3150. This moves the sensor 3110 towards the skin 3150.

Referring to FIG. 20B, the interaction between the interface element 3112 and the support surface 3134 is shown in more detail. In particular, FIG. 20B shows a point in time slightly before FIG. 20A, where the interface element 3112 has just passed the retaining element 3118.

FIG. 20B is similar to FIG. 19B, except that the housing 3102 has moved distally relative to the guide 3114. Compared to FIG. 19B, FIG. 20B shows the interface element 3112 having overcome the retaining element 3118 and moved past the retaining element 3118. In particular, the protrusion 3122 of interface element 3112 on the deflectable arm 3120 has sprung out of the recess 3126 and over the projection 3124 of the retaining element 3118 on the guide 3114. This allows the deflectable arm 3122 to move distally in order to move the housing 3102 relative to the guide 3114.

Referring back to FIG. 20A, as the deflectable arm 3120 is biased towards the guide 3114, the interface element 3112 is pushed against the support surface 3128. As the support surface 3128 is flat it does not inhibit distal movement in the same way that the retaining element 3118 did, so the interface element 3112 is free to move distally along the support surface 3128. However, because of the bias, contact between the interface element 3112 and the support surface 3128 is ensured. This keeps the guide 3114 and the housing 3102 aligned, which helps direct the sensor 3110 in a controlled manner as it approaches the skin 3150.

As the guide 3114 is held in position relative to the skin 3150 by the distal end 3116, the sensor 3110 has moved along the guide 3114 because the sensor control device 3108 has distally moved with the device carrier 3106 as part of the housing 3102. In the first embodiment, the sensor 3110 has moved part way through the hollow distal portion of the guide 3114 towards the skin 3150. A portion of the distal portion of the guide 3114 is now arranged within the housing 3102.

As the user continues to push the insertion apparatus 3100, the housing 3102 moves towards the skin 3150 and the sensor 3110 approaches the skin 3150. The interface element 3112 continues to slide along the support surface 3128. At a particular point, the sensor 3110 will come into contact with the skin 3150. The sensor 3110 will contact the skin 3150 before the adhesive patch of the sensor control device 3108 touches the skin 3150 because the sensor 3110 protrudes distally from the sensor control device 3108. This allows the sensor 3110 to be positioned at the desired penetration depth.

Because the retaining element 3118 retained the interface element 3112 in position under application of a distal force up to a threshold in the first configuration, it stored potential energy in the deflectable arm 3122. When the force overcomes the threshold, the interface element 3112 is able to overcome the retaining element 3118 and slide along the support surface 3128. Once the interface element 3112 overcomes the retaining element 3118, the stored potential energy is released. This enables the release of a large amount of kinetic energy. In particular, this provides far more kinetic energy than would otherwise be available to the housing 3102. This increases the velocity of the sensor 3110, accelerating the sensor 3110 towards the skin 3150. This provides sufficient energy for the sensor 3110 to penetrate the skin 3150.

In particular, the sensor 3110 has enough energy to penetrate the skin 3150 without the aid of a needle. Conventionally, sensors can be inserted into skin 3150 by using a needle to penetrate the skin 3150 and then feeding the sensor behind the needle. However, because of the energy of the sensor 3110 with the pointed tip in the present application, the sensor 3110 itself can penetrate the skin 3150 without the use of a needle. This allows the insertion apparatus 3100 to be needle-free. This is advantageous because the needle can be removed from the device, improving safety and user-concerns over needles.

As the housing 3102 moves distally relative to the guide 3114, the guide 3114 effectively moves up into the interior space of the cover 3104 as the overlap between the guide 3114 and the housing 3102 increases. FIG. 20A shows that as the housing 3102 moves down, the guide 3114 gets closer to the proximal end of the cover 3104. In doing so, the second interface element 3134 approaches the guide 3114, and the separation between the second interface element 3134 and the third retaining element 3138 decreases.

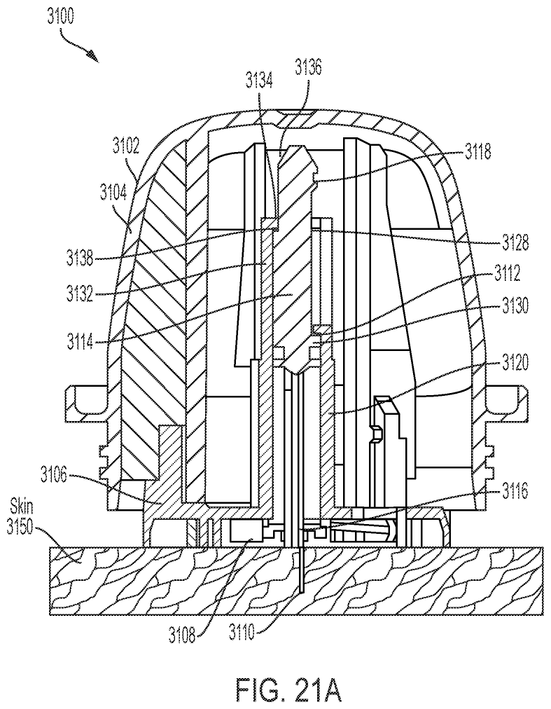

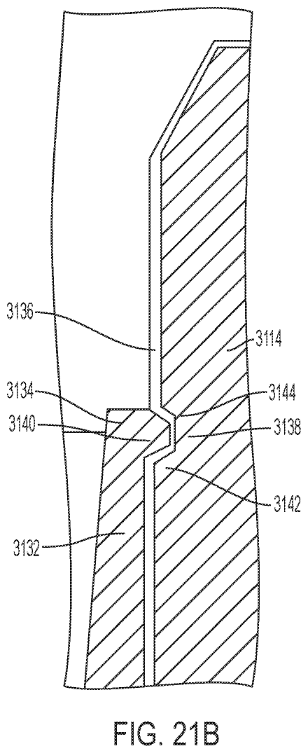

Referring to FIG. 21A, the insertion apparatus 3100 of the first embodiment is shown when the insertion apparatus 3100 is in the second configuration. FIG. 21A is similar to FIGS. 19A and 20A, except that the housing 3102 has moved further distally relative to the guide 3114. In particular, FIG. 21A shows the arrangement after the distal force applied by the user to the housing 3102 fully inserts the sensor 3110 into the skin 3150.

FIG. 21A shows that the interface element 3112 has continued to slide along the support surface 3128 until the interface element 3112 has engaged the second retaining element 3130. Because the second retaining element 3130 comprises a protrusion extending laterally out from the support surface 3128, the second retaining element 3130 prevents further distal movement of the interface element 3112. This limits distal movement of the housing 3102 relative to the guide 3114. When the interface element 3112 is engaged with the second retaining element 3130, the insertion apparatus 3100 is in the second configuration.

FIG. 21A shows the sensor 3110 inserted into the skin 3150. In particular, the sensor 3110 is fully inserted into the skin 3150. As the housing 3102 moves closer to the skin 3150, the sensor 3110 moves further along the guide 3114. In particular, the sensor 3110 moves further along the hollow distal portion of the guide 3114. FIG. 21A shows that in the second configuration, the sensor 3110 has passed out from the distal end 3116 of the guide 3114. Thus, the sensor 3110 has become exposed from the housing 3102. As the sensor 3110 is supported by the guide 3114 up until it contacts the skin 3150, the stability of insertion of the sensor 3110 is improved.

As the interface element 3112 approaches the second retaining element 3130, the sensor 3110 penetrates the skin 3150 and becomes partially inserted. The sensor 3110 continues to be inserted as the interface element 3112 continues to approach the second retaining element 3130. As the interface element 3112 reaches the second retaining element 3130, the sensor 3110 becomes fully inserted into the skin 3150. Accordingly, the length of the support surface 3128 and thus the distance between the first retaining element 3118 and the second retaining element 3130 (which defines the distance between the interface element 3112 between the first configuration and the second configuration) is selected to correspond to the distance between the sensor 3110 in the first configuration compared to the second configuration. This ensures that the sensor 3110 is fully inserted as the interface element 3112 reaches the end of the support surface 3128 and engages with the second retaining element 3130.

FIG. 21A also shows the sensor control device 3108 engaged with the skin 3150. In the second configuration, the sensor control device 3108 contacts the skin 3150. Accordingly, when the interface element 3112 engages with the second retaining element 3130 and the sensor 3110 becomes fully inserted, the sensor control device 3108 contacts the skin 3150. In other words, the sensor control device 3108 engages the skin 3150 at the same time as the interface element 3112 engages the second retaining element 3130. At this point, the sensor 3110 cannot be further inserted and the interface element 3112 cannot be further moved distally. As the sensor control device 3108 comes into contact with the skin 3150, the adhesive patch comes into contact with the skin 3150. Under the pressure of the application of the force on the insertion apparatus 3100, the adhesive patch sticks to the skin 3150. After this point, the sensor control device 3108 is released by the device carrier 3106. The adhesive patch retains the sensor control device 3108 in place on the skin 3150, with the sensor 3110 inserted into the skin 3150. The housing 3102 can then be withdrawn, leaving the sensor control device 3108 with the sensor 3110 on the skin 3150.

As the housing 3102 continues to move distally relative to the guide 3114, the guide 3114 moves further up into the interior space of the cover 3104. FIG. 21A shows that the guide 3114 is fully received into the housing 3102 in the second configuration. In the second configuration, the distal portion is withdrawn from the sensor control device 3108 so that the sensor control device 3108 can be released.

As the guide 3114 is retracted within the housing 3102 during insertion of the sensor 3110, the second interface element 3134 engages with the guide 3114. In particular, the second interface element 3134 engages with the second support surface 3136. In a similar manner to the first deflectable arm 3120, the second deflectable arm 3132 is biased towards the guide 3114. Accordingly, in the first embodiment, the second interface element 3134 engages with the second support surface 3136. Although the second support surface 3136 is flat and does not resist distal movement, the contact ensures support of the guide 3114 as the sensor 3110 approaches insertion. The combination of support of the interface element 3112 against the support surface 3128 with the support of the second interface element 3134 against the second support surface 3136 on the opposite side of the guide 3114 provides improved stability.

As the sensor 3110 becomes fully inserted, and the insertion apparatus 3100 reaches the second configuration, the second interface element 3134 engages with the third retaining element 3138 of the guide 3114.

Referring to FIG. 21B, the interaction between the second interface element 3134 and the third retaining element 3138 is shown in more detail.

In the first embodiment, the second interface element 3134 comprises a second protrusion 3140. In embodiments with the second protrusion 3140, the protrusion 3122 of the first interface element 3112 may be referred to as the first protrusion 3122. The second protrusion 3140 is arranged at the end of the second deflectable arm 3132. The second protrusion 3140 may be similar to the first protrusion 3122. For example, the second protrusion 3140 may have a proximal surface, a contact surface, and a distal surface. The proximal surface may be sloped distally towards the guide 3114 and the distal surface may be sloped distally away from the guide 3114. The contact surface may be flat.

In the first embodiment, the third retaining element 3138 comprises a third projection 3142. The third projection 3142 extends laterally out from the guide 3114 towards the second deflectable arm 3132. As the second protrusion 3140 is not designed to move beyond the projection 3142, there is no need for the third projection 3142 to be pointed, and the third projection 3142 can have a proximal surface which is sloped to restrict distal movement of the second protrusion 3140. The surface of the guide 3114 can then extend distally from the proximal surface of the third projection 3142, for example extending flat in the distal direction as shown.

In the first embodiment, the third retaining element 3138 comprises a third recess 3144. The third recess 3144 is arranged adjacent to and proximally of the third projection 3142. This means that the third recess 3144 is bounded by the third projection 3142 at the distal edge. The third recess 3144 may be similar to the first recess 3126. The third recess 3144 is configured to receive the second protrusion 3140. The third recess 3144 has a complementary shape to the second protrusion 3140. For example, the third recess 3144 has a proximal surface and a contact surface. The contact surface is adjacent to the third projection 3142. In the first embodiment, the proximal surface of the third projection is longer than the proximal surface of the third recess 3144. This aids prevention of the second interface element 3134 moving past the third retaining element 3138.

In the first configuration, the second interface element 3134 does not engage the guide 3114. As the housing 3102 moves relative to the guide 3114, the protrusion 3140 will contact the second support surface 3136. The contact surface of the protrusion 3140 will slide along the second support surface 3136 with continued movement. As the sensor 3110 becomes fully inserted and the insertion apparatus 3100 reaches the second configuration, the second interface element 3134 will engage the third retaining element 3138. In particular, the protrusion 3140 will engage with the third projection 3142 and the third recess 3144. This will prevent further distal movement of the housing 3102 relative to the guide 3114.

Moreover, because the protrusion 3140 is held within the third recess 3144, proximal movement of the housing 3102 is prevented by locking the third interface element 3130 in position. In other words, the interaction of the second interface element 3134 with the third retaining element 3138 not only further prevents distal movement, but also prevents proximal movement, avoiding dislodging the sensor 3110. This fixes the guide 3114 in the retracted position.

In some embodiments, the second interface element 3134 and the third retaining element 3138 are not provided. In some embodiments, the second retaining element 3130 may interact with the interface element 3112 to fix the guide 3114 in the second configuration. For example, the interface element 3112 may be configured to be received into a second recess of the second retaining element 3130 to prevent proximal movement. This would avoid the need for the second deflectable arm 3132, simplifying the design. Otherwise, the second recess of the second retaining element 3130 may be provided in addition to the second interface element 3134 and third retaining element 3138. This allows for greater stability by preventing proximal movement and supporting the guide 3114 from both sides. In other embodiments, the second interface element 3134 and the third retaining element 3138 may be provided instead of the second retaining element 3130, where the third projection 3142 prevents further distal movement and the second retaining element 3130 is not required. Otherwise, the second retaining element 3130 may be provided in addition to the third projection 3142. This allows for greater stability by preventing distal movement and supporting the guide 3114 from both sides.

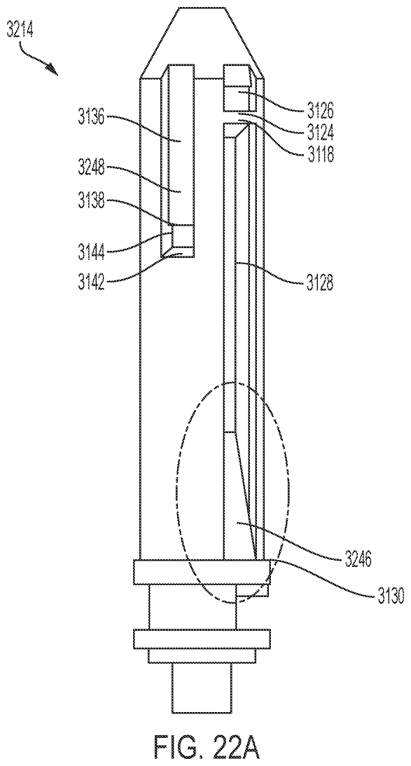

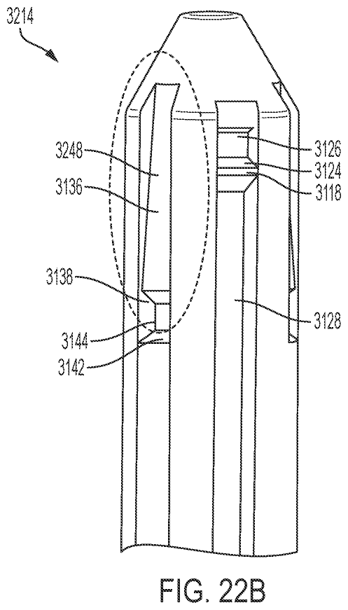

Referring to FIGS. 22A and 22B, a guide 3214 for an insertion apparatus according to a second embodiment is provided. The insertion apparatus of the second embodiment is similar to the insertion apparatus 3100 of the first embodiment, except as set out below. In particular, the insertion apparatus is similar to the insertion apparatus 3100, except that the insertion apparatus comprises a guide 3214 instead of a guide 3114 of the first embodiment. The guide 3214 of the second embodiment is similar to the guide 3114 of the first embodiment, except as set out below. FIGS. 22A and 22B show perspective views of the guide 3214 of the second embodiment. The guide 3214 has a generally cylindrical shape, where the guide 3214 has an outer circumferential surface. The guide 3214 comprises a retaining element 3118 having a projection 3124 and a recess 3126. The projection 3124 and the recess 3126 have similar cross section to that shown in FIGS. 19B and 20B. The projection 3124 and the recess 3126 have a width around the circumference of the guide 3214. The recess 3126 has a generally rectangular contact surface at the bottom.

The guide 3214 also comprises a support surface 3128. As explained above, the support surface 3128 is recessed from the outer surface of the guide 3214. The support surface 3128 joins to the distal surface of the projection 3124. The support surface 3128 is bounded by side walls along the length in the distal direction. This aids support of the interface element 3112 as it slides along the support surface 3128. Thus, the support surface 3128 is provided in a groove along the side of the guide 3214. Correspondingly, the interface element 3112 (not shown) has a width which fits within the recess 3126 and the groove of the support surface 3128.

In the second embodiment, the guide 3214 differs from the first embodiment in that the guide 3214 comprises a ramp portion 3246. The ramp portion 3246 extends from the support surface 3128 to the second retaining element 3130. The ramp portion 3246 is sloped, extending laterally outwards from the support surface 3128. The ramp portion 3246 slopes distally and laterally away from the support surface 3128. In the second embodiment, the ramp portion 3246 extends out to the outer circumference of the guide 3214. In other words, the groove of the support surface 3128 is defined in the distal direction by the distal surface of the projection 3124, the flat support surface 3128, and the ramp portion 3246, while being bounded by side walls which join the support surface 3128 within the groove to the outer circumference of the guide 3214.

During operation, as the interface element 3112 moves along the support surface 3128, it approaches the ramp portion 3246. At the ramp portion 3246, the interface element 3112 engages with the ramp portion 3246. This increases the surface area in contact with the interface element 3112. The angled surface provides an outward force against the interface element 3112 without fully preventing distal movement. This increases friction and reduces the speed of the interface element 3112. Therefore, the ramp portion 3246 acts to slow the interface element 3112 as it approaches the second retaining element 3130. This improves stability for insertion of the sensor 3110 because the movement of the sensor 3110 is more controlled. This is particularly useful as the sensor 3110 is approaching the skin 3150. This prevents buckling, bouncing, or deflecting of the sensor 3110. Accordingly, in the second embodiment, the ramp portion 3246 is arranged so that the ramp portion 3246 engages the interface element 3112 as the sensor 3110 approaches the skin 3150. In other words, while the sensor 3110 is being inserted into the skin 3150, the interface element 3112 is engaged with the ramp portion 3246 for greater control. However, the length of the support surface 3128 before the ramp portion 3246 is set so that the sensor 3110 can build up sufficient speed for needle-free insertion, taking into account the slowing at the ramp portion 3246.

In the second embodiment, the ramp portion 3246 is a flat, angled surface. In other embodiments, the ramp portion 3246 may comprise a plurality of sections having different angles (e.g., increasing angles) to incrementally slow the sensor 3110. In other embodiments, the ramp portion 3246 may be curved so that the angle changes (e.g., continuously) along its length. For example, the ramp portion 3246 may be a concave surface. This allows the sensor 3110 to be gradually and continuously slowed. The shape of the ramp portion 3246 can be adjusted to allow for the deceleration of the sensor 3110 to be controlled as desired.

In the second embodiment, the second retaining element 3130 is arranged at the end of the ramp portion 3246. In the second embodiment, the second retaining element 3130 comprises a second projection in the form of a lip around the guide 3214. The lip is a circumferential lip around the guide 3214, towards a distal end of the proximal portion of the guide 3214. The lip has a larger diameter than the outer circumferential surface of the guide 3214. The second retaining element 3130 provides a surface to prevent further distal movement of the interface element 3130 once it reaches the end of the support surface 3128 (in this case including the ramp portion 3246) and once the sensor 3110 is fully inserted and the sensor control device 3108 contacts the skin 3150.

In other embodiments, the second retaining element 3130 may further comprise a second recess arranged proximally of the second projection for retaining the interface element 3112, as set out above.

Similarly to the first embodiment, the guide 3214 also comprises a third retaining portion 3138 having a third projection 3142 and a third recess 3144. The third retaining portion 3138 is configured to receive the second interface element 3134 after it passes over the second support surface 3136 as the sensor 3110 is inserted.

In the second embodiment, the guide 3214 additionally differs from the first embodiment in that the guide 3214 also comprises a second ramp portion 3248. The second ramp portion 3248 is arranged on the second support surface 3136. In particular, the second ramp portion 3248 is arranged adjacent to and proximally of the third retaining portion 3138. The second ramp portion 3248 is sloped, extending laterally inwards from the proximal surface of the recess 3144.

Referring to FIG. 22B, the second ramp portion 3248 is shown in more detail. The second ramp portion 3248 slopes proximally and laterally towards from the center of the guide 3214 away from the outer circumference of the guide 3214. In the second embodiment, the outer surface of the guide 3214 tapers at the proximal end to a narrower diameter. The second ramp portion 3248 extends to meet the tapered portion at the proximal end, part way along the tapered length. This makes it easier for the second interface element 3134 to enter the groove of the second ramp portion 3248. The second ramp portion 3248 then slopes outwards in the distal direction. This acts to slow the second interface element 3134. In combination with the ramp portion 3246, this can be used to slow the sensor 3110 as it approaches the skin 3150. The second ramp portion 3248 then meets the proximal surface of the recess 3144, which then allows the second interface element 3134 to fit into the recess 3144 where it can be held. The proximal surface of the recess 3144 can then prevent proximal movement of the second interface element 3134, while the projection 3142 forming the distal surface of the recess 3144 can prevent distal movement.

In some embodiments, the guide 3214 may comprise the ramp portion 3246 and/or the second ramp portion 3248. In other words, some embodiments may provide the ramp portion 3246 with or without the second ramp portion 3248, and some embodiments may provide the second ramp portion 3248 with or without the ramp portion 3246.

Example Embodiments of an Insertion Apparatus with Sheath

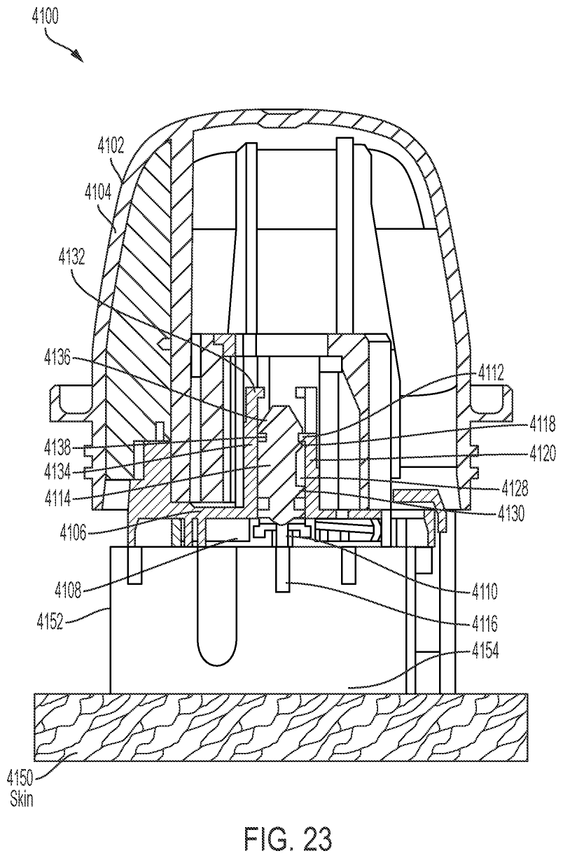

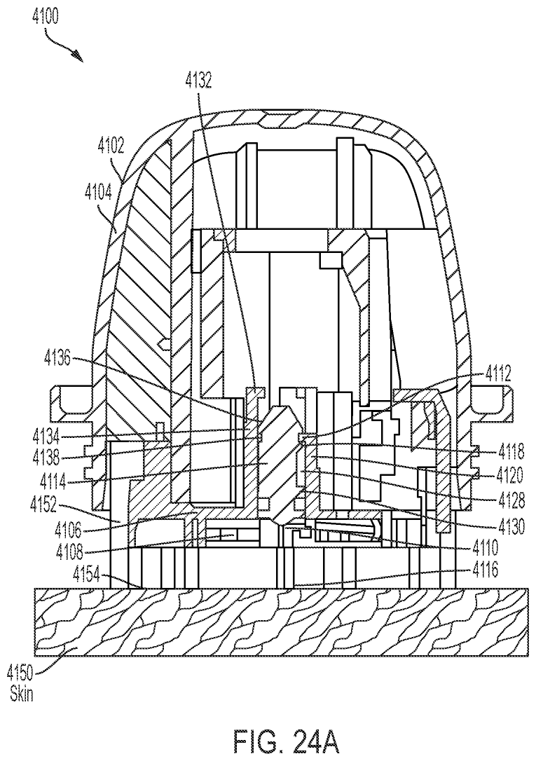

Referring to FIGS. 23 to 26B, according to a third embodiment of the present disclosure, an insertion apparatus 4100 is provided.

Referring in particular to FIG. 23, the insertion apparatus 4100 is shown. The insertion apparatus 4100 of the third embodiment is similar to the insertion apparatus 3100 of the first embodiment as described above in relation to FIGS. 19A to 21B, except as set out below. In particular, the insertion apparatus 4100 is similar to the insertion apparatus 3100, except that the insertion apparatus 4100 further comprises a sheath 4152. In some embodiments, the sheath 4152 may be similar to the sheath 704 as described herein, and the sheath 4152 may comprise one or more features described in relation to the sheath 704.

The sheath 4152 comprises a proximal portion and a distal portion. The proximal portion is narrower and is arranged to house the guide 4114. The distal portion has a larger diameter which is generally similar (but slightly smaller) to the diameter of the cover 4104. Referring to FIG. 23, the sheath 4152 is partially exposed. In particular, the distal portion of the sheath 4152 extends beyond the cover 4104 in the distal direction.

Referring to FIG. 23, the sheath 4152 is shown in a first configuration. The first configuration of the third embodiment is different from the first configuration of the first embodiment because the guide 4114 is not in contact with the skin 4150, and instead the sheath 4152 is in contact with the skin 4150. In the first configuration of the third embodiment, the interface element 4112 of the deflectable arm 4120 of the device carrier 4106 of the housing 4104 engages with the retaining element 4118 of the guide 4114.

Referring to FIG. 23, the sheath 4152 is in contact with the skin 4150. The sheath 4152 comprises a distal end 4154. The distal end 4154 of the sheath 4152 is in contact with the skin 4150. The distal end 4154 abuts the skin 4150 so that as the user presses the insertion apparatus 4100 against the skin 1450, the distal end 4154 engages the skin 4150.

The sheath 4152 is slidably connected to the housing 4102. In this manner, the sheath 4152 can slide in the axial direction relative to the housing 4102. In particular, the housing 4102 can slide over the sheath 4152 to cover more of the distal portion of the sheath 4152.

In more detail, the housing 4102 comprises a sheath interface element and the sheath 4152 comprises a sheath retaining element. In some examples, the sheath interface element is arranged on the cover 4106 and/or the device carrier 4106. The sheath retaining element of the sheath 4152 engages with the sheath interface element of the housing 4102. Under a distal force applied to the housing 4102, the distal end 4154 of the sheath 4152 engages the skin 4150, which provides a restoring force to resist distal movement. The sheath retaining element thus retains the sheath interface element in position. This allows the distal movement of the housing 4102 to be resisted relative to the sheath 4152. In particular, when a distal force is applied to the housing 4102, such as by a user pushing on the cover 4104, distal movement of the housing 4102 is resisted, when the distal force is below a threshold. Potential energy can be stored in response to this resistance. For instance, potential energy may be stored in the sheath interface element and/or the sheath retaining element.

In the third embodiment, the guide 4114 is similar to the guide of the first embodiment, except that the guide 4114 is shorter. The distal portion of the guide 4114 is short because it does not need to be in contact with the skin 4150 in the first configuration, because instead the sheath 4152 engages the skin 4150. In the third embodiment, the length of the guide 4114 is also less due to a shorter supporting surface 4128. This allows the guide 4114 to be housed within the sheath 4152 without the sheath 4152 interfering with the cover 4104. In other embodiments, the supporting surface 4128 may be longer, and for example the cover 4104 may be longer.

In the first configuration, the interface element 4112 of the deflectable arm 4120 of the device carrier 4106 engages with the retaining element 4118 of the guide 4114. This retains the guide 4114 in position relative to the housing 4102, which is in turn held in position relative to the sheath 4152.

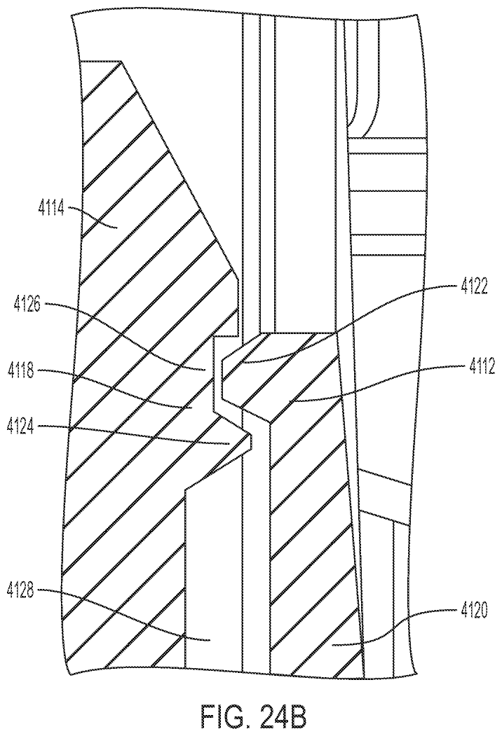

Referring to FIG. 24A, when the distal force on the housing 4102 exceeds the threshold force, the sheath interface element can overcome the sheath retaining element, and the housing 4102 may be free to move distally relative to the sheath 4152. The stored potential energy can be released to allow the housing 4102 to accelerate towards the skin 4150. This allows the user to trigger movement of the housing 4102 under application of a predefined force.

FIG. 24A shows the housing 4102 moved over the sheath 4152 towards the skin 4150. As the housing 4102 is slidably moveable relative to the sheath 4152, the housing 4102 slides over the sheath 4152, covering a portion of the sheath 4152. As the housing 4102 moves distally, the device carrier 4106 with the sensor control device 4108 and the sensor 4110 also move distally closer to the skin 4150. Due to the interface element 4112 of the deflectable arm 4120 of the device carrier 4106 engaging with the retaining element 4118 of the guide 4114, the device carrier 106 pulls the guide 4114 distally as the housing 4104 moves. As shown in FIG. 24A, this brings the guide 4114 into contact with the skin 4150. In particular, the distal end 4116 of the guide 4114 engages the skin 4150. In the third embodiment, the sensor 4110 is not yet in contact with the skin 4150, meaning that the guide 4114 has a sensor support channel longer than a length of the sensor 4110.

FIG. 24A shows a second configuration of the third embodiment. The second configuration of the third embodiment is similar to the first configuration of the first embodiment in that the guide 4114 contacts the skin 4150. In particular, the sheath 4152 has now collapsed into the housing 4104, exposing the skin 4150 to the guide 4114. The guide 4114 is now armed ready for firing, similar to the first configuration of the first embodiment. The insertion apparatus 4100 has moved from the first configuration to the second configuration under the distal force to collapse the sheath 4152.

Referring to FIG. 24B, the interaction between the interface element 4112 and the retaining element 4118 is shown in more detail. It will be appreciated that this is similar to the first configuration of the first embodiment. In particular, the interface element 4112 of the deflectable arm 4120 of the device carrier 4106 is engaged with the retaining element 4118 of the guide 4114. The interface element 4112 has a protrusion 4112 which engages with a recess 4126 and a projection 4124 of the retaining element 4118, in a similar manner to the first embodiment.

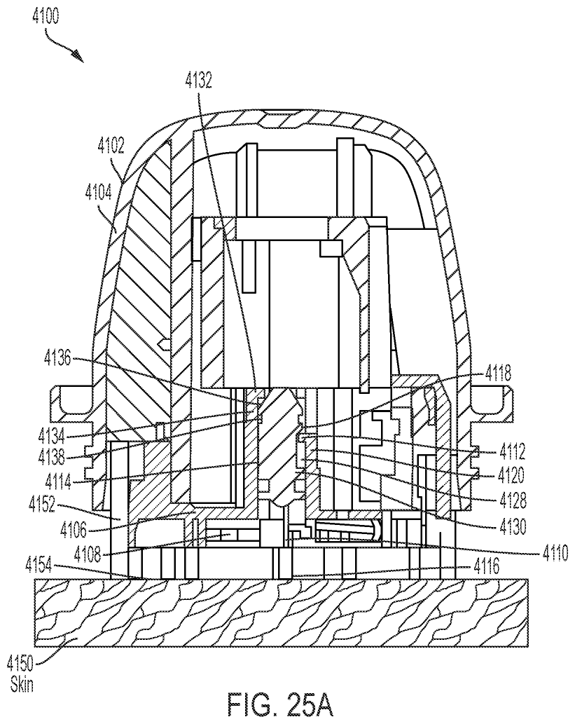

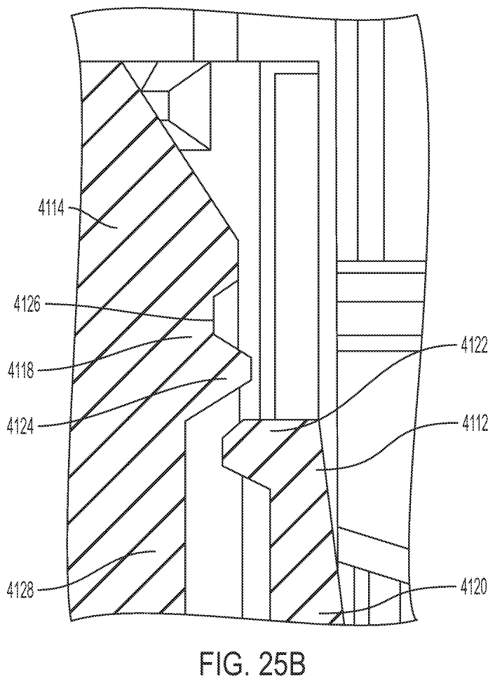

Referring to FIG. 25A, the insertion apparatus 4100 is shown moving from the second configuration to a third configuration. In particular, the interface element 4112 has overcome the retaining element 4118 in response to the distal force exceeding the threshold force. This is similar to the first embodiment. In summary, under the distal force on the housing 4102, potential energy can be stored as the guide 4114 pushes against the skin 4150 and the retaining element 4118 prevents distal movement of the interface element 4112. Once the force exceeds the threshold, the interface element 4112 moves past the retaining element 4118 and is free to move distally. The interface element 4112 moves along the supporting surface 4128, which can support the sensor 4110 for insertion. The housing 4102 is thus free to move, and the sensor 4110 within the sensor control device 4108 accelerates towards the skin 4150.

Referring to FIG. 25B, the interface element 4112 is shown having overcome the retaining element 4118. In particular the protrusion 4122 of the interface element 4112 has snapped out of the recess 4126 and over the projection 4124 to move distally.

Referring back to FIG. 25A, the second interface element 4132 of the second deflectable arm 4134 of the device carrier 4106 approaches engagement with the guide 4114 similar to the first embodiment. In particular, the second interface element 4132 comes into contact with the second supporting surface 4136 as it approaches the third retaining element 4138. This can further support the sensor 4110 for insertion.

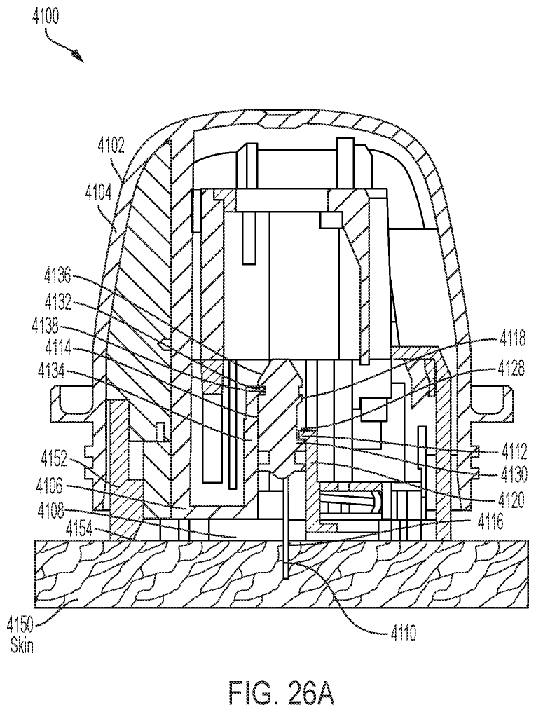

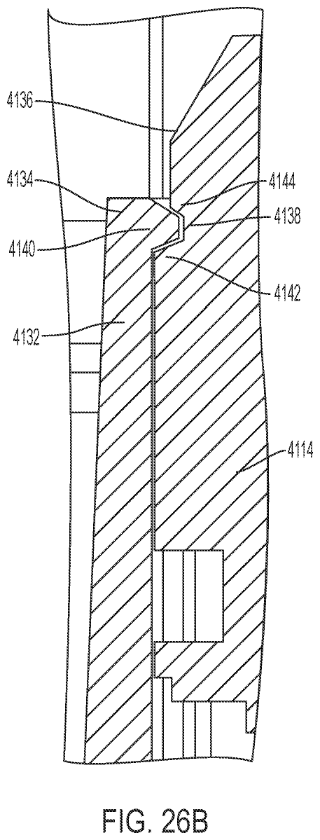

Referring to FIG. 26A, the insertion apparatus 4100 is shown in the third configuration. The third configuration of the third embodiment is similar to the second configuration of the first embodiment in that the sensor 4110 is inserted into the skin 4150. In particular, in the third configuration, the housing 4102 has continued to move distally until the interface element 4112 engages with the second retaining element 4130 which prevents further distal movement. At this point, the sensor control device 4108 engages with the skin 4150. The sensor 4110 is now fully inserted. This is similar to the first embodiment. The sensor control device 4108 can now be released on the skin 4150 with the sensor 4110 inserted.

Referring to FIG. 26B, the second interface element 4132 of the second deflectable arm 4134 engages with the third retaining element 4138. The protrusion 4140 of the second interface element 4134 engages with the recess 4144 and the projection 4142 of the third retaining element 4138. This prevents proximal movement of the housing 4102, preventing dislodging of the sensor 4110.

In other embodiments, the sheath 4152 of the third embodiment may be provided with other embodiments than the first embodiment. For example, the sheath 4152 of the third embodiment may be provided with a guide having a ramp portion and/or second ramp portion similar to the guide 3214 of the second embodiment. Other features described in relation to other embodiments may be readily applied to the third embodiment and vice versa.

Example Embodiments of an Insertion Apparatus with Sheath

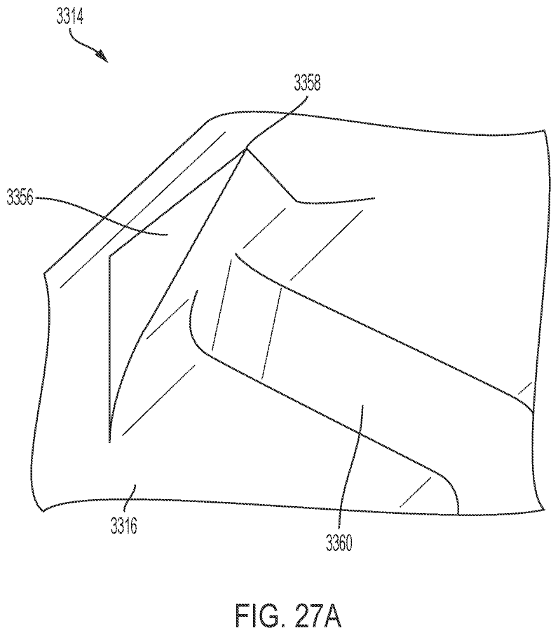

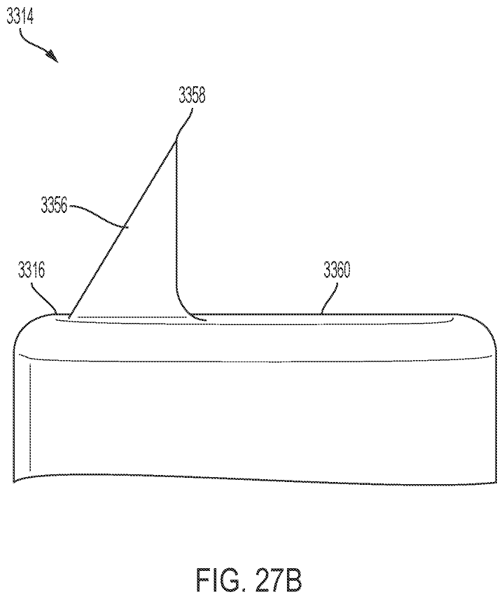

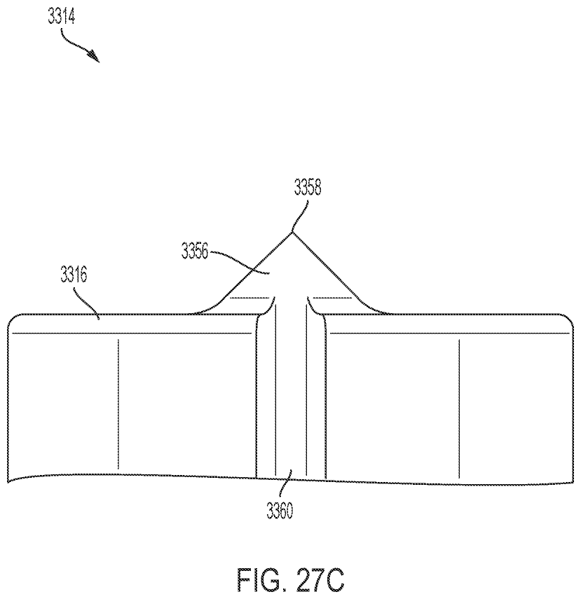

Referring to FIGS. 27A to 27C, according to a fourth embodiment of the present disclosure, an insertion apparatus is provided.

Referring in particular to FIG. 27A, the guide 3314 for an insertion apparatus is shown. The insertion apparatus of the fourth embodiment is similar to the insertion apparatus 3100 of the first embodiment, except as set out below. In particular, the insertion apparatus of the fourth embodiment is similar to the insertion apparatus 3100, except that the insertion apparatus of the fourth embodiment comprises a guide 3314 instead of the guide 3114 of the first embodiment. The guide 3314 of the fourth embodiment is similar to the guide 3114 of the first embodiment or the guide 3214 of the second embodiment, except as set out below.

FIG. 27A shows a perspective view of the underside of the guide 3314. The guide 3314 comprises a distal end 3316, shown in FIG. 27A. The guide 3314 comprises a projection element 3356 at the distal end 3316. In particular, the projection element 3356 extends from the distal end 3316. In other words, the projection element 3356 protrudes from the distal end 3316. The projection element 3356 may otherwise be referred to as a protuberance, an extension element, or a projection. The projection element 3356 extends from the distal end 3316 at least in the direction of the length of the guide 3314. FIG. 27A shows the projection element 3356 extending upwards from the distal end 3316. Accordingly, when the guide 3314 is oriented for use against the skin, such as that shown in FIG. 19A, the projection element 3356 will extend downwards beyond the surface of the distal end 3316. In the fourth embodiment, the projection element 3356 is integral with the guide 3314. In other embodiments, the projection element 3356 may be provided separately and attached to the guide 3314.

The projection element 3356 comprises a tip 3358. In the fourth embodiment, the tip 3358 is a pointed tip. In other words, the projection element 3356 extends to a pointed tip 3358. The tip 3358 may otherwise be described as sharp. In the fourth embodiment, the projection element 3356 has a triangular shape. In particular, the projection element 3356 has a triangular cross section forming a pyramidal shape. The projection element 3356 protrudes from the distal end 3316 at a base having a triangular footprint, converging to a point at the tip 3358. In other words, the projection element 3356 may be considered to be a spike.

The guide 3114 comprises a sensor support channel 3360. The sensor support channel 3360 is configured to support the sensor for insertion, as described herein. The edges of the sensor support channel 3360 can support the sensor for insertion. In the fourth embodiment, the sensor support channel 3360 comprises a recess into the side of the guide 3314. The sensor support channel 3360 has a depth into the guide 3314 from the side of the guide 3314, where the depth is defined by two opposing side faces. The sensor support channel 3360 also has a width between the two opposing side faces, defined by a back face. The depth is larger than the width in the fourth embodiment. The sensor support channel 3360 has edges defined by the side faces and the back face, which extend through the guide 3314 for supporting the sensor, where the edges are generally parallel to a length of the guide 3314 and perpendicular to the distal end 3316.

An end of the sensor support channel 3360 is arranged at the distal end 3316. In the fourth embodiment, the projection element 3356 is arranged adjacent to the end of the sensor support channel 3360. In particular, the tip 3358 is arranged at a position aligned with an edge of the sensor support channel 3360. In more detail, the projection element 3356 is arranged adjacent to the back face of the sensor support channel 3360. The tip 3358 is aligned with the edge of the sensor support channel 3360 of the back face, in a direction parallel to the edge of the sensor support channel 3360 and perpendicular to the surface of the distal end 3316. The projection element 3356 extends along the width of the sensor support channel 3360.

Referring to FIG. 27B, the guide 3314 is shown from a side perspective. The projection element 3356 protrudes from the distal end 3316. In particular, the projection element 3356 has a triangular cross section from the side. The cross section is generally a right-angled triangle shape, where the side (distal side) furthest from the sensor support channel 3360 is angled (sloped) from the base towards the tip 3358 in the direction towards the sensor support channel 3360. Meanwhile, the side (proximal side) closest to the sensor support channel 3360 is generally perpendicular to the surface of the distal end 3316. As shown in FIG. 27B, this provides the tip 3358 adjacent to the sensor support channel 3360. As shown in FIG. 27A, this is achieved by three flat triangular-shaped surfaces defining a pyramid shape, but other shapes are possible.

Referring to FIG. 27C, the guide 3314 is shown from a side perspective rotated 90° from FIG. 27B, from the front looking into the sensor support channel 3360. The projection element 3356 is shown protruding from the distal end 3316, with a triangular cross section. The tip 3358 is aligned with the center of the sensor support channel 3360 along a width of the edge of the sensor support channel 3360 such that the tip 3358 is generally aligned with a midpoint of the back face of the sensor support channel 3360.

During operation, when the guide 3314 engages the skin, the distal end 3316 is configured to contact the skin. As the projection element 3356 protrudes beyond the distal end 3316, the projection element 3356 deforms the skin. In particular, the projection element 3356 pushes against the skin, and the skin bends to permit receiving the projection element 3356. In the fourth embodiment, the projection element 3356 bends the skin without penetrating it. This creates a small indent in the surface of the skin. Due to the triangular shape of the projection element 3356, the deepest point of the indent corresponds to the position of the pointed tip 3358 of the projection element 3356. When the sensor is inserted (for example, using a mechanism as set out herein), the sensor is supported by the sensor support channel 3360, and continues to be supported by the proximal surface of the projection element 3356. The projection element 3356 guides the sensor from the distal end 3316 to the skin. In particular, the projection element 3356 directs the sensor specifically to the bottom of the indent caused by the projection element 3356. The pointed tip 3358 defines a precise location at the bottom of the indent of the skin. The projection element 3356 thus guides the sensor to the end of the projection element 3356 and to the pointed tip 3358. At this point, the sensor can enter the skin at the end of the indent. In this manner, the projection element 3356 can accurately aim the sensor to a precise location defined by the point tip 3358. This improves the aiming of the sensor, and support of the sensor during insertion. This also inhibits buckling of the sensor. This is particularly useful when the sensor is inserted without use of a sharp (e.g., a sharpless applicator, or a sensor configured to penetrate the skin, such as described herein). In alternative embodiments, the projection element 3356 may penetrate the skin. In particular, the projection element 3656 pushes against the skin, and the tip may enter through the skin. Because the tip of the projection element 3656 penetrates the skin before the sensor is inserted, this aids insertion of the sensor. This is particularly useful when the sensor is inserted without use of a sharp (e.g., a sharpless applicator, or a sensor configured to penetrate the skin, such as described herein).

In alternative embodiments, the projection element 3356 may have different shapes. For example, the projection element 3356 may terminate in a point using shapes other than a pyramid. Alternatively, the projection element 3356 may be located in a different position, such as adjacent to a side face of the sensor support channel 3360.

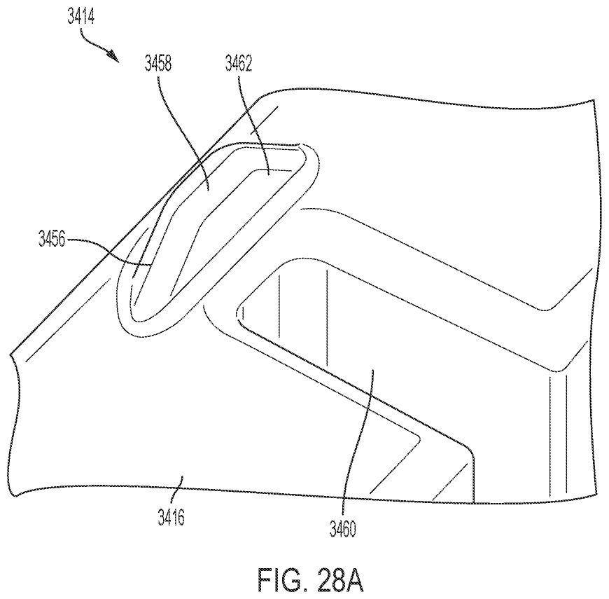

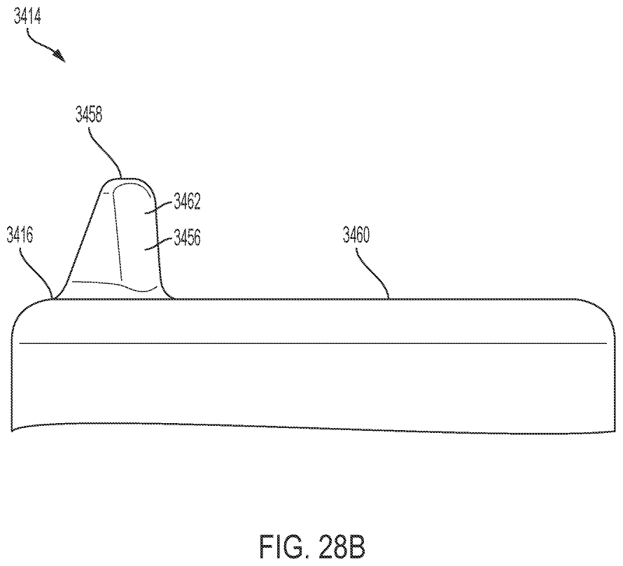

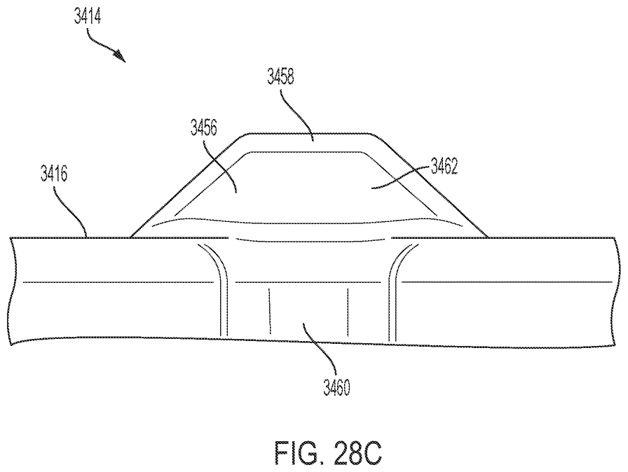

Referring to FIGS. 28A to 28C, according to a fifth embodiment of the present disclosure, an insertion apparatus is provided.

Referring in particular to FIG. 28A, the guide 3414 for an insertion apparatus is shown. The insertion apparatus of the fifth embodiment is similar to the insertion apparatus 3100 of the first embodiment, except as set out below. The guide 3414 of the fifth embodiment is similar to the guide 3314 of the fourth embodiment, except as set out below.

FIG. 28A is from a similar perspective to FIG. 27A. The guide 3414 comprises a distal end 3416 and a projection element 3456. In the fifth embodiment, the projection element 3456 extends along one side of the sensor support channel 3460, in particular along the edge adjacent to the back face. The projection element 3456 is adjacent to the end of the sensor support channel 3460. The projection element 3456 has a planar cross section from above. The projection element 3456 is in the form of a wall 3462. The wall 3462 extends along the edge of the end of the back face of the sensor support channel 3460. The wall 3462 has a height defined by angled surfaces at each end of the wall 3462. The angled surfaces slope from the base of the wall 3462 towards the middle of the wall until they reach the tip 3458. The tip 3458 is the top of the wall 3462. The tip 3458 is generally flat (i.e., the wall 3462 does not change height along the length of the tip 3458). The tip 3458 has a length along the length of the wall 3462 adjacent to the back face of the sensor support channel 3460. In other words, the projection element 3456 forms a trapezium shape, where the angled surfaces meet a top surface defining a tip 3458.

In contrast to the tip 3356 of the fourth embodiment, the tip 3458 of the fifth embodiment is a rounded surface of the top of the projection element 3456. The tip 3458 extends along the top of the projection element 3456, along the top of the wall. The tip 3458 extends in a plane parallel to the distal end 3416 and perpendicular to the sensor support channel 3460. In other words, the tip 3458 is not a point, but has a length defining a top of the wall.

Referring to FIG. 28B, the guide 3414 is shown from a side perspective. FIG. 28B is from a similar perspective to FIG. 27B. The projection element 3456 extends along one side of the sensor support channel 3460, adjacent to the back face.

The side (proximal side) of the projection element 3456 closest to the sensor support channel 3460 is generally flat and perpendicular to the surface of the distal end 3416 and parallel to an edge of the sensor support channel 3460, but this may be angled in other examples. The side (distal side) of the projection element 3456 furthest from the sensor support channel 3460 is angled towards the sensor support channel 3460 as it approaches the tip 3458. This forms a tip 3458 narrower than the base. In the fifth embodiment, the tip 3458 is curved and does not form a sharp point.

Referring to FIG. 28C, the guide 3414 is shown from a side perspective rotated 90° from FIG. 28B. FIG. 28C is from a similar perspective to FIG. 28C. This shows the trapezium cross section of the projection element 3458. The length of the wall 3458 is longer than the width of the sensor support channel 3460 (the dimension of the back face), and the length of the tip 3458 (being the top of the wall 3458) is approximately equal to the width of the sensor support channel 3460 (the width of the back face), but in other examples other sizes are possible.

During operation, when the guide 3414 engages the skin, the distal end 3416 is configured to contact the skin. As the projection element 3456 protrudes beyond the distal end 3416, the projection element 3456 deforms the skin. In particular, the projection element 3456 pushes against the skin, and the skin bends to permit receiving the projection element 3456. In the fifth embodiment, the projection element 3456 bends the skin without penetrating it. This creates a small indent in the surface of the skin. The tip 3458 of the fifth embodiment is provided to tighten the skin in the region in which the sensor is to be inserted. In particular, the tip 3458 adjacent to the sensor support channel 3460 stretches the skin along a length corresponding to the length of the tip 3458. This tightens the skin adjacent to the end of the sensor support channel 3460, meaning the skin is tightened near the region where the sensor will be inserted. When the sensor is inserted (for example, using a mechanism as set out herein), the sensor insertion is aided by the tightened skin. This improves the insertion of the sensor. This is particularly useful when the sensor is inserted without use of a sharp (e.g., a sharpless applicator, or a sensor configured to penetrate the skin, such as described herein).

In other embodiments, the position of the projection element 3456 may be different. For example, the projection element 3456 may be positioned on a different edge of the sensor support channel 3460 (such as adjacent to a side face), or on more than one side. In other examples, the guide may comprise more than one projection element 3456, such as on opposing sides of the sensor support channel 3460 for stretching the skin across the end of the sensor support channel 3460.

In other embodiments, the wall of the fifth embodiment may be combined with another projection element such as the spike of the fourth embodiment. This can combine the functionality of aiming the sensor and stretching the skin.

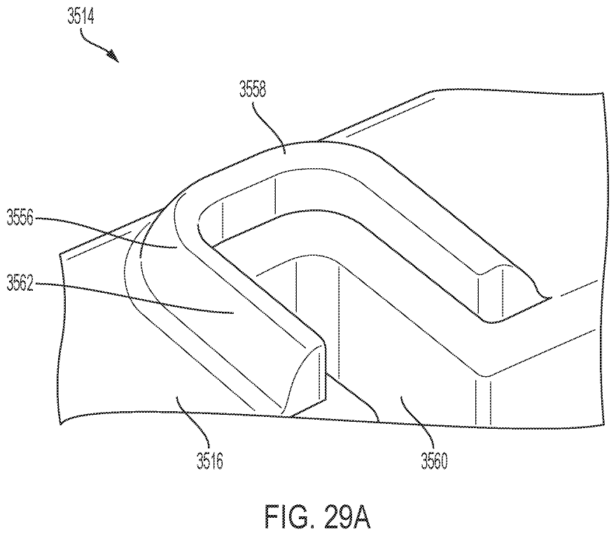

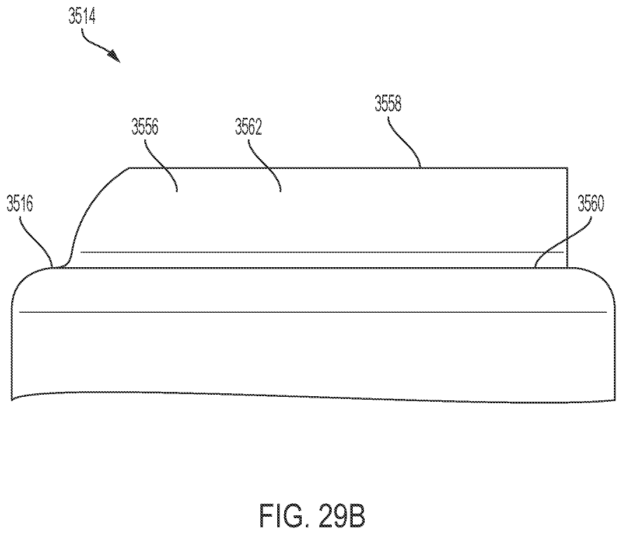

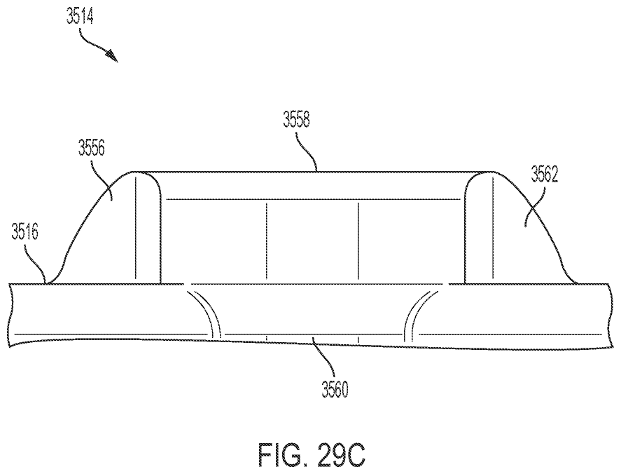

Referring to FIGS. 29A to 29C, according to a sixth embodiment of the present disclosure, an insertion apparatus is provided.

Referring in particular to FIG. 29A, the guide 3514 for an insertion apparatus is shown. The insertion apparatus of the sixth embodiment is similar to the insertion apparatus 3100 of the first embodiment, except as set out below. The guide 3514 of the sixth embodiment is similar to the guide 3414 of the fifth embodiment, except as set out below.

FIG. 29A is from a similar perspective to FIGS. 27A and 28A. The guide 3514 comprises a distal end 3516 and a projection element 3556. In the sixth embodiment, the projection element 3556 is in the form of a wall 3562. The wall 3562 extends around an edge of the sensor support channel 3560. As shown in FIG. 29A, the wall 3562 extends around three faces of the sensor support channel 3560. In particular, the wall 3562 extends along edges of the back face and the two opposing side faces of the sensor support channel 3560. Thus, the wall 3562 has a U-shape, or a horseshoe shape. The wall 3562 is adjacent to the end of the sensor support channel 3560. In other examples, the wall 3562 may extend around a sensor support channel 3562 in the form of a hole, in which case the wall 3562 may be a closed wall, such as a circular shape (e.g., circle, oval, etc.).

The wall 3562 terminates at a tip 3558. In a similar manner to the fifth embodiment, the tip 3558 is a rounded surface of the top of the wall 3562. In other words, the tip 3558 extends along the top of the wall 3562. The tip 3558 extends in a plane parallel to the distal end 3516 and perpendicular to the sensor support channel 3560.

Referring to FIG. 29B, the guide 3514 is shown from a side perspective. FIG. 29B is from a similar perspective to FIGS. 27B and 28B. The projection element 3556 has a wall 3562 extending around the sensor support channel 3560.

The side (proximal side) of the wall 3562 closest to the sensor support channel 3560 is flat and perpendicular to the surface of the distal end 3516 and parallel to an edge of the sensor support channel 3560. The side (distal side) of the wall 3562 furthest from the sensor support channel 3560 is rounded and curves towards the sensor support channel 3560 as it approaches the tip 3558. This forms a tip 3558 of the wall 3562 narrower than the base. In the fifth embodiment, the tip 3558 is curved and does not form a sharp point.

Referring to FIG. 29C, the guide 3514 is shown from a side perspective rotated 90° from FIG. 29B. FIG. 29C is from a similar perspective to FIGS. 27C and 28C. The projection element 3556 surrounds the three faces of the sensor support channel 3560. FIG. 29B shows the curve in the distal side of the wall 3562. The rounded tip 3558 of the wall 3562 is also shown.

During operation, when the guide 3514 engages the skin, the distal end 3516 is configured to contact the skin. As the projection element 3556 protrudes beyond the distal end 3516, the projection element 3556 deforms the skin. In particular, the projection element 3556 pushes against the skin, and the skin bends to permit receiving the projection element 3556. In the fifth embodiment, the projection element 3556 bends the skin without penetrating it. This creates a small indent in the surface of the skin. Due to the wall 3562 of the projection element 3556, the indent in the skin is generally U-shaped. This forms the indent at three sides corresponding to the faces of the sensor support channel 3560. In a similar manner to the fifth embodiment, the tip 3558 of the sixth embodiment is provided to tighten the skin in the region in which the sensor is to be inserted. In particular, the wall 3562 stretches the skin in the U-shape, which tightens the skin between opposing sides of the U-shape. This tightens the skin over the end of the sensor support channel, meaning the skin is tightened across the region where the sensor will be inserted. When the sensor is inserted (for example, using a mechanism as set out herein), the sensor insertion is aided by the tightened skin. This improves the insertion of the sensor. This is particularly useful when the sensor is inserted without use of a sharp (e.g., a sharpless applicator, or a sensor configured to penetrate the skin, such as described herein).

The stretching is improved because the skin is stretched from two locations. In other embodiments, a similar effect may be achieved by providing a plurality of discrete projection elements arranged over the distal end. For example, the guide may comprise at least two projection elements, such as at opposing edges of the sensor support channel. These can provide anchor points for stretching the skin. In some examples, the plurality of projection elements may be discrete projection elements such as bumps, or non-continuous wall sections.

In other embodiments, the wall of the sixth embodiment may be combined with another projection element such as the spike of the fourth embodiment or the wall of the fifth embodiment. This can combine the functionality of aiming the sensor and stretching the skin.

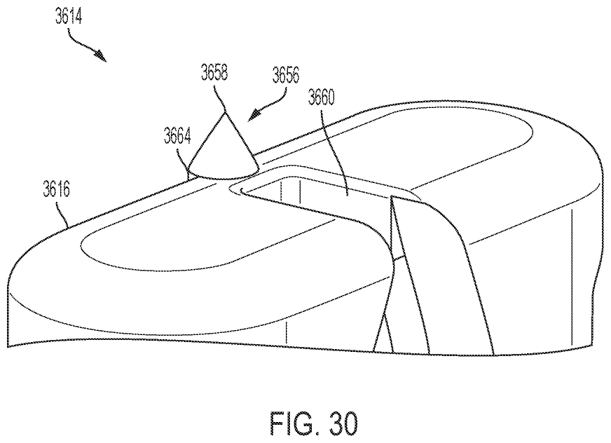

Referring to FIG. 30, according to a seventh embodiment of the present disclosure, an insertion apparatus is provided.

Referring in particular to FIG. 30, the guide 3614 for an insertion apparatus is shown. The insertion apparatus of the seventh embodiment is similar to the insertion apparatus 3100 of the first embodiment, except as set out below. The guide 3614 of the seventh embodiment is similar to the guide 3314 of the fourth embodiment, the guide 3414 of the fifth embodiment, and the guide 3514 of the sixth embodiment, except as set out below.

FIG. 30 is from a similar perspective to FIGS. 27A to 29A. The guide 3614 comprises a distal end 3616 and a projection element 3656. In the seventh embodiment, the projection element 3656 is in the form of an end or tip 3658 of a needle 3664. In particular, the needle 3664 is a thin needle. In the seventh embodiment, the needle 3664 is an acupuncture needle. The projection element 3656 is arranged at an edge of the sensor support channel 3660. As shown in FIG. 30, the projection element 3656 is arranged adjacent to the back face of the sensor support channel 3660. In some embodiments, the end or tip of the needle 3664 is a separate component and is attached to the guide 3614. In other embodiments, the needle 3664 is integral with the guide 3614. The projection element 3656 terminates at a tip 3658. In a similar manner to the fourth embodiment, the tip 3658 is a pointed tip. The tip 3658 is the tip of the needle 3664. In some embodiments, the projection element 3656 may comprise a needle 3664 extending adjacent to the back face of the sensor support channel 3660.

During operation, when the guide 3614 engages the skin, the distal end 3616 is configured to contact the skin. As the projection element 3656 protrudes beyond the distal end 3616, the projection element 3656 interacts with the skin. In contrast to the fourth to sixth embodiments, the projection element 3656 of the seventh embodiment is configured to pre-pierce and penetrate the skin. In particular, the needle 3664 pushes against the skin, and the tip 3658 enters through the skin. Because the needle 3664 penetrates the skin before the sensor is inserted, this aids insertion of the sensor. This is particularly useful when the sensor is inserted without use of a sharp (e.g., a sharpless applicator, or a sensor configured to penetrate the skin, such as described herein).

In other embodiments, a plurality of needles 3664 may be provided. For example, needles 3664 may be provided in different locations, such as on different sides of the sensor support channel 3660.

In other embodiments, the needle may be combined with another projection element such as the spike of the fourth embodiment and/or the wall of the fifth or sixth embodiment. This can combine the functionality of aiming the sensor and/or stretching the skin with pre-piercing the skin.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the inventive scope of the claims by features, functions, steps, or elements that are not within that scope.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible. The embodiments described herein are restated and expanded upon in the following paragraphs without explicit reference to the figures.

Systems, devices, and methods are provided for inserting a sensor into skin of a subject. The insertion apparatus includes a sensor control device, a housing, and a guide for guiding insertion of a sensor into the skin. The housing may have an interface element, a cover, and a device carrier configured to releasably retain the sensor control device. The guide may be configured to support at least a portion of the sensor, and includes a retaining element. In a first configuration, the retaining element is configured to engage with the interface element to resist distal movement of the housing relative to the guide. In a second configuration, the interface element is configured to disengage from the retaining element to distally move the housing relative to the guide and insert the sensor into the skin. The insertion apparatus is configured to move from the first to the second configuration in response to a distal force on the housing.

In many embodiments, an insertion apparatus for inserting a sensor into skin of a subject includes a sensor control device comprising a sensor, wherein the sensor is configured to penetrate the skin of the subject; a housing comprising a cover and a device carrier coupled to the cover, wherein the device carrier is configured to releasably retain the sensor control device, and wherein the housing comprises an interface element; and a guide for guiding insertion of the sensor into the skin, wherein the guide is configured to support at least a portion of the sensor, wherein the guide comprises a distal end configured to engage the skin to allow distal movement of the housing relative to the guide, and wherein the guide comprises a retaining element; wherein the insertion apparatus has a first configuration in which the retaining element of the guide is configured to engage with the interface element of the housing to resist distal movement of the housing relative to the guide; and wherein the insertion apparatus is configured to move from the first configuration to a second configuration in response to a distal force on the housing above a threshold force, in which the interface element is configured to disengage from the retaining element to distally move the housing relative to the guide and insert the sensor into the skin.

In some embodiments, the retaining element and the interface element are configured to store potential energy whilst resisting distal movement of the housing in response to a distal force on the housing below the threshold force. In some embodiments, the stored potential energy is released when the interface element disengages from the retaining element, accelerating the sensor towards the skin.

In some embodiments, the distal end of the guide is configured to act against the skin in response to the distal force to provide a restoring force for engaging the retaining element with the interface element when the distal force is below the threshold force, and for allowing the housing to distally move relative to the guide when the distal force is above the threshold force.

In some embodiments, the interface element is arranged on the device carrier.

In some embodiments, the interface element is arranged on the cover.

In some embodiments, the retaining element comprises a protrusion extending laterally from the guide, and wherein the interface element is arranged proximally of the protrusion in the first configuration and the protrusion is configured to resist distal movement of the interface element.

In some embodiments, the interface element is configured to deflect over the protrusion to move to the second configuration when the distal force is above the threshold force.

In some embodiments, the retaining element comprises a recess adjacent to and proximally of the protrusion, wherein the recess is configured to receive the interface element in the first configuration to resist distal movement of the interface element.

In some embodiments, the interface element is configured to deflect out of the recess to move to the second configuration when the distal force is above the threshold force.

In some embodiments, the housing comprises a deflectable arm, and wherein the interface element comprises an end of the deflectable arm.

In some embodiments, the guide comprises a second retaining element, wherein the second retaining element is arranged distally of the retaining element, and wherein the second retaining element, when in the second configuration, is configured to engage with the interface element to resist distal movement of the housing relative to the guide.

In some embodiments, an axial distance between the retaining element and the second retaining element corresponds to a distance for movement of the housing for insertion of the sensor into the skin.

In some embodiments, the guide comprises a third retaining element, wherein the housing comprises a second interface element, and wherein the third retaining element, when in the second configuration, is configured to engage with the second interface element to resist proximal movement of the housing relative to the guide.

In some embodiments, the housing comprises a second deflectable arm, and wherein the second interface element comprises an end of the second deflectable arm.

In some embodiments, the apparatus also includes a biasing element configured to bias the interface element against the guide.

In some embodiments, the guide comprises a supporting surface arranged distally of the retaining element, and wherein the interface element is configured to contact and slide along the supporting surface when moving to the second configuration.

In some embodiments, the supporting surface comprises at least a straight portion parallel to an axial direction.

In some embodiments, the supporting surface comprises a ramp portion angled relative to an axial direction and extending laterally outwards.

In some embodiments, the ramp portion is configured to increase surface contact between the guide and the interface element.

In some embodiments, the ramp portion is arranged at a distal portion of the supporting surface.

In some embodiments, the ramp portion is configured to contact the interface element as the sensor is inserted into the skin.

In some embodiments, the ramp portion is configured to contact the interface element as the sensor extends beyond the guide when moving into the second configuration.

In some embodiments, the ramp portion is arranged adjacent to and proximally of the second retaining element.

In some embodiments, the guide comprises a second supporting surface arranged proximally of the third retaining element, and wherein the second interface element is configured to contact and slide along the second supporting surface when moving to the second configuration.

In some embodiments, the second supporting surface comprises a second ramp portion angled relative to an axial direction and extending laterally inwards.

In some embodiments, the second ramp portion is configured to increase surface contact between the guide and the second interface element.

In some embodiments, the second ramp portion is arranged at a proximal portion of the second supporting surface.

In some embodiments, the second ramp portion is configured to contact the second interface element as the sensor is inserted into the skin.

In some embodiments, the second ramp portion is configured to contact the second interface element as the sensor extends beyond the guide when moving into the second configuration.

In some embodiments, the second ramp portion is arranged adjacent to and proximally of the third retaining element.

In some embodiments, the guide comprises a sensor support channel, and wherein the sensor is arranged to distally move through the sensor support channel within the guide in order to guide insertion of the sensor into the skin.

In some embodiments, at least a portion of the guide is arranged to extend through the device carrier.

In some embodiments, in the second configuration, the sensor control device contacts the skin, and the device carrier is configured to release the sensor control device on the skin.

In some embodiments, the apparatus also includes a sheath slidably moveable relative to the housing, and wherein the sheath comprises a distal end configured to engage the skin.

In some embodiments, the housing is configured to distally move relative to the sheath in response to a second distal force on the housing.

In some embodiments, distally moving the housing relative to the sheath comprises moving the distal end of the guide into contact with the skin before the movement from the first configuration to the second configuration.

In some embodiments, the sensor comprises a pointed tip configured to penetrate the skin.

In some embodiments, the sensor comprises a free length portion configured to provide rigidity to facilitate insertion of the sensor into the skin.

In some embodiments, the sensor is an in vivo analyte sensor configured to measure an analyte level in a bodily fluid of the subject.

In some embodiments, the guide comprises a projection element extending from the distal end of the guide, and wherein the projection element is configured to interact with the skin when the distal end engages the skin.

In some embodiments, the projection element is arranged adjacent to an end of the sensor support channel at the distal end.

In some embodiments, the projection element is configured to deform the skin.

In some embodiments, the projection element comprises a pointed tip.

In some embodiments, the projection element comprises a rounded tip.

In some embodiments, the projection element comprises a wall arranged to extend along the distal surface of the guide.

In some embodiments, the projection element is configured to pre-penetrate the skin to aid insertion of the sensor.

In some embodiments, the projection element comprises a needle.

In some embodiments, the needle is an acupuncture needle.

In many embodiments, an insertion apparatus for inserting a sensor into skin of a subject includes a sensor control device comprising a sensor; a housing comprising a cover and a device carrier coupled to the cover, wherein the device carrier is configured to releasably retain the sensor control device; and a guide for guiding insertion of the sensor into the skin, wherein the guide comprises a sensor support channel configured to support at least a portion of the sensor, wherein the guide comprises a distal end configured to engage the skin to allow distal movement of the housing relative to the guide; wherein the guide comprises a projection element extending from the distal end of the guide, and wherein the projection element is configured to interact with the skin when the distal end engages the skin.

In some embodiments, the projection element is arranged adjacent to an end of the sensor support channel at the distal end.

In some embodiments, the projection element is configured to interact with the skin such that the projection element passes through a plane defining a position of the skin.

In some embodiments, the projection element is configured to deform the skin.

In some embodiments, the projection element is configured to deform the skin to assist aiming of the sensor to aid insertion of the sensor.

In some embodiments, the projection element comprises a pointed tip.

In some embodiments, the pointed tip terminates at a position aligned with an edge of the sensor support channel.

In some embodiments, the projection element comprises a triangular cross section.

In some embodiments, the projection element is configured to deform the skin to stretch the skin to aid insertion of the sensor.

In some embodiments, the projection element comprises a rounded tip.

In some embodiments, a tip of the projection element is defined by a surface extending in a plane parallel to the distal surface of the guide.

In some embodiments, a tip of the projection element is arranged at least at two positions in a plane parallel to the distal surface of the guide.

In some embodiments, the projection element is arranged at least at opposing sides of the sensor support channel.

In some embodiments, the projection element comprises a wall arranged to extend along the distal surface of the guide.

In some embodiments, the wall extends around at least part of the edge of the end of the sensor support channel.

In some embodiments, the wall extends around at least three sides of the end of the sensor support channel.

In some embodiments, the projection element is configured to pre-penetrate the skin to aid insertion of the sensor.

In some embodiments, the projection element comprises a needle.

In some embodiments, the needle is an acupuncture needle.

In some embodiments, the sensor is configured to penetrate the skin.

In some embodiments, the sensor comprises a pointed tip configured to penetrate the skin.

In some embodiments, the sensor comprises a free length portion configured to provide rigidity to facilitate insertion of the sensor into the skin.

In some embodiments, the insertion apparatus comprises a sharp configured to penetrate the skin.

In some embodiments, the sensor is an in vivo analyte sensor configured to measure an analyte level in a bodily fluid of the subject.

### CLAUSES

Exemplary embodiments are set out in the following numbered clauses.

Clause 1. An insertion apparatus for inserting a sensor into skin of a subject, comprising:
a sensor control device comprising a sensor, wherein the sensor is configured to penetrate the skin of the subject;
a housing comprising a cover and a device carrier coupled to the cover, wherein the device carrier is configured to releasably retain the sensor control device, and wherein the housing comprises an interface element; and
a guide for guiding insertion of the sensor into the skin, wherein the guide is configured to support at least a portion of the sensor, wherein the guide comprises a distal end configured to engage the skin to allow distal movement of the housing relative to the guide, and wherein the guide comprises a retaining element;
wherein the insertion apparatus has a first configuration in which the retaining element of the guide is configured to engage with the interface element of the housing to resist distal movement of the housing relative to the guide; and
wherein the insertion apparatus is configured to move from the first configuration to a second configuration in response to a distal force on the housing above a threshold force, in which the interface element is configured to disengage from the retaining element to distally move the housing relative to the guide and insert the sensor into the skin.

Clause 2. The insertion apparatus of clause 1, wherein the retaining element and the interface element are configured to store potential energy whilst resisting distal movement of the housing in response to a distal force on the housing below the threshold force.

Clause 3. The insertion apparatus of clause 2, wherein the stored potential energy is released when the interface element disengages from the retaining element, accelerating the sensor towards the skin.

Clause 4. The insertion apparatus of any preceding clause, wherein the distal end of the guide is configured to act against the skin in response to the distal force to provide a restoring force for engaging the retaining element with the interface element when the distal force is below the threshold force, and for allowing the housing to distally move relative to the guide when the distal force is above the threshold force.

Clause 5. The insertion apparatus of any preceding clause, wherein the interface element is arranged on the device carrier.

Clause 6. The insertion apparatus of any of clauses 1 to 4, wherein the interface element is arranged on the cover.

Clause 7. The insertion apparatus of any preceding clause, wherein the retaining element comprises a protrusion extending laterally from the guide, and wherein the interface element is arranged proximally of the protrusion in the first configuration and the protrusion is configured to resist distal movement of the interface element.

Clause 8. The insertion apparatus of clause 7, wherein the interface element is configured to deflect over the protrusion to move to the second configuration when the distal force is above the threshold force.

Clause 9. The insertion apparatus of clause 7 or 8, wherein the retaining element comprises a recess adjacent to and proximally of the protrusion, wherein the recess is configured to receive the interface element in the first configuration to resist distal movement of the interface element.

Clause 10. The insertion apparatus of clause 9, wherein the interface element is configured to deflect out of the recess to move to the second configuration when the distal force is above the threshold force.

Clause 11. The insertion apparatus of any preceding clause, wherein the housing comprises a deflectable arm, and wherein the interface element comprises an end of the deflectable arm.

Clause 12. The insertion apparatus of any preceding clause, wherein the guide comprises a second retaining element, wherein the second retaining element is arranged distally of the retaining element, and wherein the second retaining element, when in the second configuration, is configured to engage with the interface element to resist distal movement of the housing relative to the guide.

Clause 13. The insertion apparatus of clause 12, wherein an axial distance between the retaining element and the second retaining element corresponds to a distance for movement of the housing for insertion of the sensor into the skin.

Clause 14. The insertion apparatus of clause 13, wherein the guide comprises a third retaining element, wherein the housing comprises a second interface element, and wherein the third retaining element, when in the second configuration, is configured to engage with the second interface element to resist proximal movement of the housing relative to the guide.

Clause 15. The insertion apparatus of clause 14, wherein the housing comprises a second deflectable arm, and wherein the second interface element comprises an end of the second deflectable arm.

Clause 16. The insertion apparatus of any preceding clause, further comprising a biasing element configured to bias the interface element against the guide.

Clause 17. The insertion apparatus of any preceding clause, wherein the guide comprises a supporting surface arranged distally of the retaining element, and wherein the interface element is configured to contact and slide along the supporting surface when moving to the second configuration.

Clause 18. The insertion apparatus of clause 17, wherein the supporting surface comprises at least a straight portion parallel to an axial direction.

Clause 19. The insertion apparatus of clause 17 or 18, wherein the supporting surface comprises a ramp portion angled relative to an axial direction and extending laterally outwards.

Clause 20. The insertion apparatus of clause 19, wherein the ramp portion is configured to increase surface contact between the guide and the interface element.

Clause 21. The insertion apparatus of clause 19 or 20, wherein the ramp portion is arranged at a distal portion of the supporting surface.

Clause 22. The insertion apparatus of any of clauses 19 to 21, wherein the ramp portion is configured to contact the interface element as the sensor is inserted into the skin.

Clause 23. The insertion apparatus of any of clauses 19 to 22, wherein the ramp portion is configured to contact the interface element as the sensor extends beyond the guide when moving into the second configuration.

Clause 24. The insertion apparatus of any of clauses 19 to 22 when dependent on clause 12, wherein the ramp portion is arranged adjacent to and proximally of the second retaining element.

Clause 25. The insertion apparatus of clause 14, wherein the guide comprises a second supporting surface arranged proximally of the third retaining element, and wherein the second interface element is configured to contact and slide along the second supporting surface when moving to the second configuration.

Clause 26. The insertion apparatus of clause 25, wherein the second supporting surface comprises a second ramp portion angled relative to an axial direction and extending laterally inwards.

Clause 27. The insertion apparatus of clause 26, wherein the second ramp portion is configured to increase surface contact between the guide and the second interface element.

Clause 28. The insertion apparatus of clause 26 or 27, wherein the second ramp portion is arranged at a proximal portion of the second supporting surface.

Clause 29. The insertion apparatus of any of clauses 26 to 28, wherein the second ramp portion is configured to contact the second interface element as the sensor is inserted into the skin.

Clause 30. The insertion apparatus of any of clauses 26 to 29, wherein the second ramp portion is configured to contact the second interface element as the sensor extends beyond the guide when moving into the second configuration.

Clause 31. The insertion apparatus of any of clauses 26 to 30, wherein the second ramp portion is arranged adjacent to and proximally of the third retaining element.

Clause 32. The insertion apparatus of any preceding clause, wherein the guide comprises a sensor support channel, and wherein the sensor is arranged to distally move through the sensor support channel within the guide in order to guide insertion of the sensor into the skin.

Clause 33. The insertion apparatus of any preceding clause, wherein at least a portion of the guide is arranged to extend through the device carrier.

Clause 34. The insertion apparatus of any preceding clause, wherein, in the second configuration, the sensor control device contacts the skin, and the device carrier is configured to release the sensor control device on the skin.

Clause 35. The insertion apparatus of any preceding clause, further comprising a sheath slidably moveable relative to the housing, and wherein the sheath comprises a distal end configured to engage the skin.

Clause 36. The insertion apparatus of clause 35, wherein the housing is configured to distally move relative to the sheath in response to a second distal force on the housing.

Clause 37. The insertion apparatus of clause 36, wherein distally moving the housing relative to the sheath comprises moving the distal end of the guide into contact with the skin before the movement from the first configuration to the second configuration.

Clause 38. The insertion apparatus of any preceding clause, wherein the sensor comprises a pointed tip configured to penetrate the skin.

Clause 39. The insertion apparatus of any preceding clause, wherein the sensor comprises a free length portion configured to provide rigidity to facilitate insertion of the sensor into the skin.

Clause 40. The insertion apparatus of any preceding clause, wherein the sensor is an in vivo analyte sensor configured to measure an analyte level in a bodily fluid of the subject.

Clause 41. The insertion apparatus of any preceding clause, wherein the guide comprises a projection element extending from the distal end of the guide, and wherein the projection element is configured to interact with the skin when the distal end engages the skin.

Clause 42. The insertion apparatus of clause 41 when dependent on clause 32, wherein the projection element is arranged adjacent to an end of the sensor support channel at the distal end.

Clause 43. The insertion apparatus of clause 41 or 42, wherein the projection element is configured to deform the skin.

Clause 44. The insertion apparatus of any of clauses 41 to 43, wherein the projection element comprises a pointed tip.

Clause 45. The insertion apparatus of any of clauses 41 to 44, wherein the projection element comprises a rounded tip.

Clause 46. The insertion apparatus of any of clauses 41 to 45, wherein the projection element comprises a wall arranged to extend along the distal surface of the guide.

Clause 47. The insertion apparatus of any of clauses 41 to 46, wherein the projection element is configured to pre-penetrate the skin to aid insertion of the sensor.

Clause 48. The insertion apparatus of any of clauses 41 to 47, wherein the projection element comprises a needle.

Clause 49. The insertion apparatus of clause 48, wherein the needle is an acupuncture needle.

Clause 50. An insertion apparatus for inserting a sensor into skin of a subject, comprising:
a sensor control device comprising a sensor;
a housing comprising a cover and a device carrier coupled to the cover, wherein the device carrier is configured to releasably retain the sensor control device; and
a guide for guiding insertion of the sensor into the skin, wherein the guide comprises a sensor support channel configured to support at least a portion of the sensor, wherein the guide comprises a distal end configured to engage the skin to allow distal movement of the housing relative to the guide;
wherein the guide comprises a projection element extending from the distal end of the guide, and wherein the projection element is configured to interact with the skin when the distal end engages the skin.

Clause 51. The insertion apparatus of clause 50, wherein the projection element is arranged adjacent to an end of the sensor support channel at the distal end.

Clause 52. The insertion apparatus of clause 50 or 51, wherein the projection element is configured to interact with the skin such that the projection element passes through a plane defining a position of the skin.

Clause 53. The insertion apparatus of any of clauses 50 to 52, wherein the projection element is configured to deform the skin.

Clause 54. The insertion apparatus of clause 54, wherein the projection element is configured to deform the skin to assist aiming of the sensor to aid insertion of the sensor.

Clause 55. The insertion apparatus of any of clauses 50 to 54, wherein the projection element comprises a pointed tip.

Clause 56. The insertion apparatus of clause 55, wherein the pointed tip terminates at a position aligned with an edge of the sensor support channel.

Clause 57. The insertion apparatus of any of clauses 50 to 56, wherein the projection element comprises a triangular cross section.

Clause 58. The insertion apparatus of any of clauses 50 to 57, wherein the projection element is configured to deform the skin to stretch the skin to aid insertion of the sensor.

Clause 59. The insertion apparatus of any of clauses 50 to 58, wherein the projection element comprises a rounded tip.

Clause 60. The insertion apparatus of any of clauses 50 to 59, wherein a tip of the projection element is defined by a surface extending in a plane parallel to the distal surface of the guide.

Clause 61. The insertion apparatus of any of clauses 50 to 60, wherein a tip of the projection element is arranged at least at two positions in a plane parallel to the distal surface of the guide.

Clause 62. The insertion apparatus of any of clauses 50 to 61, wherein the projection element is arranged at least at opposing sides of the sensor support channel.

Clause 63. The insertion apparatus of any of clauses 50 to 62, wherein the projection element comprises a wall arranged to extend along the distal surface of the guide.

Clause 64. The insertion apparatus of clause 63, wherein the wall extends around at least part of the edge of the end of the sensor support channel.

Clause 65. The insertion apparatus of clause 64, wherein the wall extends around at least three sides of the end of the sensor support channel.

Clause 66. The insertion apparatus of any of clauses 50 to 65, wherein the projection element is configured to pre-penetrate the skin to aid insertion of the sensor.

Clause 67. The insertion apparatus of any of clauses 50 to 66, wherein the projection element comprises a needle.

Clause 68. The insertion apparatus of any of clauses 50 to 67, wherein the needle is an acupuncture needle.

Clause 69. The insertion apparatus of any of clauses 50 to 68, wherein the sensor is configured to penetrate the skin.

Clause 70. The insertion apparatus of clause 69, wherein the sensor comprises a pointed tip configured to penetrate the skin.

Clause 71. The insertion apparatus of clause 50 or 70, wherein the sensor comprises a free length portion configured to provide rigidity to facilitate insertion of the sensor into the skin.

Clause 72. The insertion apparatus of any of clauses 50 to 71, wherein the insertion apparatus comprises a sharp configured to penetrate the skin.

Clause 73. The insertion apparatus of any of clauses 50 to 72, wherein the sensor is an in vivo analyte sensor configured to measure an analyte level in a bodily fluid of the subject.

1. An insertion apparatus for inserting a sensor into skin of a subject, comprising:
a sensor control device comprising a sensor, wherein the sensor is configured to penetrate the skin of the subject;
a housing comprising a cover and a device carrier coupled to the cover, wherein the device carrier is configured to releasably retain the sensor control device, and wherein the housing comprises an interface element; and
a guide for guiding insertion of the sensor into the skin, wherein the guide is configured to support at least a portion of the sensor, wherein the guide comprises a distal end configured to engage the skin to allow distal movement of the housing relative to the guide, and wherein the guide comprises a retaining element;
wherein the insertion apparatus has a first configuration in which the retaining element of the guide is configured to engage with the interface element of the housing to resist distal movement of the housing relative to the guide; and
wherein the insertion apparatus is configured to move from the first configuration to a second configuration in response to a distal force on the housing above a threshold force, in which the interface element is configured to disengage from the retaining element to distally move the housing relative to the guide and insert the sensor into the skin.

2. The insertion apparatus of claim 1, wherein the retaining element and the interface element are configured to store potential energy whilst resisting distal movement of the housing in response to a distal force on the housing below the threshold force.

3. The insertion apparatus of claim 2, wherein the stored potential energy is released when the interface element disengages from the retaining element, accelerating the sensor towards the skin.

4. The insertion apparatus of claim 1, wherein the distal end of the guide is configured to act against the skin in response to the distal force to provide a restoring force for engaging the retaining element with the interface element when the distal force is below the threshold force, and for allowing the housing to distally move relative to the guide when the distal force is above the threshold force.

5. The insertion apparatus of claim 1, wherein the interface element is arranged on the device carrier.

6. The insertion apparatus of claim 1, wherein the interface element is arranged on the cover.

7. The insertion apparatus of claim 1, wherein the retaining element comprises a protrusion extending laterally from the guide, and wherein the interface element is arranged proximally of the protrusion in the first configuration and the protrusion is configured to resist distal movement of the interface element.

8. The insertion apparatus of claim 7, wherein the interface element is configured to deflect over the protrusion to move to the second configuration when the distal force is above the threshold force.

9. The insertion apparatus of claim 7, wherein the retaining element comprises a recess adjacent to and proximally of the protrusion, wherein the recess is configured to receive the interface element in the first configuration to resist distal movement of the interface element.

10. The insertion apparatus of claim 9, wherein the interface element is configured to deflect out of the recess to move to the second configuration when the distal force is above the threshold force.

11. The insertion apparatus of claim 1, wherein the housing comprises a deflectable arm, and wherein the interface element comprises an end of the deflectable arm.

12. The insertion apparatus of claim 1, wherein the guide comprises a second retaining element, wherein the second retaining element is arranged distally of the retaining element, and wherein the second retaining element, when in the second configuration, is configured to engage with the interface element to resist distal movement of the housing relative to the guide.

13. The insertion apparatus of claim 12, wherein an axial distance between the retaining element and the second retaining element corresponds to a distance for movement of the housing for insertion of the sensor into the skin.

14. The insertion apparatus of claim 13, wherein the guide comprises a third retaining element, wherein the housing comprises a second interface element, and wherein the third retaining element, when in the second configuration, is configured to engage with the second interface element to resist proximal movement of the housing relative to the guide.

15. The insertion apparatus of claim 14, wherein the housing comprises a second deflectable arm, and wherein the second interface element comprises an end of the second deflectable arm.

16. The insertion apparatus of claim 1, further comprising a biasing element configured to bias the interface element against the guide.

17-34. (canceled)

35. The insertion apparatus of claim 1, further comprising a sheath slidably moveable relative to the housing, and wherein the sheath comprises a distal end configured to engage the skin.

36. The insertion apparatus of claim 35, wherein the housing is configured to distally move relative to the sheath in response to a second distal force on the housing.

37. The insertion apparatus of claim 36, wherein distally moving the housing relative to the sheath comprises moving the distal end of the guide into contact with the skin before the movement from the first configuration to the second configuration.

38. The insertion apparatus of claim 1, wherein the sensor comprises a pointed tip configured to penetrate the skin.

39. The insertion apparatus of claim 1, wherein the sensor comprises a free length portion configured to provide rigidity to facilitate insertion of the sensor into the skin.

40-73. (canceled)
